(19) United States
(12) Patent Application Publication
(10) Pub. No.: US 2023/0293540 A1
(43) Pub. Date: Sep. 21, 2023

(54) ANTICOAGULANT COMPOUNDS, METHODS AND DEVICES FOR OPHTHALMIC USE

(71) Applicant: Elixir Medical Corporation, Milpitas, CA (US)

(72) Inventors: John Yan, Los Gatos, CA (US); Xiaoxia Zheng, Fremont, CA (US); Vinayak D. Bhat, Cupertino, CA (US); Motasim Sirhan, Los Altos, CA (US); Margaret Wong, Redwood City, CA (US); Blaine Bueche, San Mateo, CA (US)

(73) Assignee: Elixir Medical Corporation, Milpitas, CA (US)

(21) Appl. No.: 18/179,160

(22) Filed: Mar. 6, 2023

Related U.S. Application Data
(63) Continuation of application No. PCT/US2021/049964, filed on Sep. 10, 2021.
(60) Provisional application No. 63/193,013, filed on May 25, 2021.
(60) Provisional application No. 63/077,170, filed on Sep. 11, 2020.

(51) Int. Cl. A61K 31/5377; A61K 9/00; A61K 31/4545; A61K 47/10; A61P 27/02

(52) U.S. Cl. CPC A61K 31/5377; A61K 9/0019; A61K 9/0048; A61K 31/4545; A61K 47/10; A61P 27/02

(57) ABSTRACT

Devices, formulations, compositions, and methods are provided for inhibiting an inflammatory ophthalmic disease or eye condition in a patient. A therapeutic composition comprising a direct factor Xa inhibitor and/or a direct factor II inhibitor is provided. A therapeutically effective dose of the therapeutic composition is delivered to a site of the inflammatory ophthalmic disease or condition in the patient’s eye(s). The therapeutic composition may be formulated for delivery to the patient by via injection, an implant, an eye drop, an emulsion, a suspension, an ointment, a nanomicelle, a nanoparticle, a nanosuspension, a liposome, an in-situ gel, a contact lens, or a microneedle or the like. The therapeutic composition may include one or more additional therapeutically active substances and/or one or more additional pharmaceutical agents. The one or more additional therapeutically active substances and/or one or more additional pharmaceutical agents may be delivered together with the direct factor Xa inhibitor and/or direct factor IIa inhibitor, or separately from the direct factor Xa inhibitor and/or direct factor IIa inhibitor.

### CROSS-REFERENCE TO RELATED APPLICATION

This application is a continuation of PCT Application No. PCT/US2021/49964 (Attorney Docket No. 32016-724.601), filed Sep. 10, 2021, which claims the benefit of U.S. Provisional Application No. 63/193,013 (Attorney Docket No. 32016-722.102), filed May 25, 2021, and of U.S. Provisional Application No. 63/077,170 (Attorney Docket No. 32016-724.101), filed Sep. 11, 2020, each of which is incorporated herein by reference for all purposes in its entirety.

### BACKGROUND OF THE INVENTION

1. Field of the Invention. The present disclosure relates generally to anticoagulants and derivatives thereof and their use in therapeutic applications, and in particular with their use in ophthalmic applications.

As we age or as a result of eye condition, inflammatory disease of the eye or other disease conditions such as hypertension or diabetes, can manifest itself in the eye in various forms including but not limited to wet macular degeneration, dry macular degeneration, diabetic retinopathy, glaucoma, dry eye, etc. In some instances, drug therapies are available and have had some success in treating symptoms of the condition or disease but often fail to address the underlying inflammatory disease condition. For example, anti-angiogenic agents can be used to treat wet macular degeneration, and about 50% of patients respond to such therapy, but such agents do not adequately address the inflammatory cause of the disease condition and in many cases further deterioration of visual acuity or the eye occurs.

Systemic drugs have had limited success in addressing these conditions or in long term effectiveness to address such conditions.

It is therefore an objective of the present invention to provide improved compositions, methods, and devices for treating wet macular degeneration, dry macular degeneration, diabetic retinopathy, glaucoma, dry eye, and other inflammatory conditions in the eye. At least some of these objectives will be met by the inventions described and claimed herein.

2. Background Art. Ophthalmic solutions incorporating anti-inflammatory and anti-coagulant substances are described in WO2005/077344 and WO2007056218. Other background art includes US6,500,855; US8,409,272; US8,946,219; US2003/0158120; US2005/0064006; US2009/0075949; US2010/0003542; US2010/0130543; US2010/0184729; US2018/0000490; EP1849434; CA2464290; and WO2013/007840.

The subject matter of this application is also related to that of the following commonly owned applications: PCT/US2021/34108 (Attorney Docket No. 32016-720.601), filed May 25, 2021 and PCT/US2021/44414 (Attorney Docket No. 32016-723.601), filed Aug. 3, 2021, the full disclosures of which are incorporated herein by reference.

### SUMMARY OF THE DISCLOSURE

In many cases, inflammation, chronic inflammation, formation or increase of fluid build-up, fibrosis, thrombin, cell apoptosis, and/or fibrin/clot formation occur, thereby damaging the eye or ocular tissues. The condition continues to upregulate the inflammatory and/or immune reaction pathway and generate excessive amounts of one of thrombin, fibrin, immune reaction which in turn can lead to or increasing the likelihood of morbidities.

There is still a need to develop specialized therapeutic compositions for ophthalmic applications which can prevent, reduce, dissolute, resolve, inhibit, or eliminate one or more of fibrin and/or clot, thrombin, immune reaction, glaucoma, and inflammation and reduce associated eye morbidities and mortalities/blindness.

It would therefore be desirable to provide a formulation or a devices that locally deliver one or more agents to inhibit or dissolute thrombin/clot formation, inflammation, immune response, and glaucoma and optionally other types of biologically active agents (e.g., anti-fibrotic agents, other anti-inflammatory agents, antiviral agents, antibiotic agents, anti-proliferative agents, immune suppressant agents, etc.), to the eye to inhibit, reduce, and/or prevent coagulation, fibrin formation, thrombin formation, inflammation, glaucoma, immune reaction, and/or clot formation in the eye(s), and/or fibrosis of the ocular tissue, and/or inflammation of the ocular fluid or tissue, and/or to treat viral infections, and/or to treat bacterial infections.

In one aspect, a method of treating an inflammatory ophthalmic condition or disease in a patient is provided. The method comprises providing a therapeutic composition comprising a direct factor Xa inhibitor and/or a direct factor IIa inhibitor; and delivering a therapeutically effective dose of the therapeutic composition to a site of the inflammatory ophthalmic condition or disease in the patient’s eye.

In some examples, the therapeutically effective dose of the therapeutic composition is effective to suppress or prevent initiation, progression, or relapses of disease, including the progression of established disease.

In some examples, delivering the therapeutically effective dose of the therapeutic composition comprises administering the therapeutically effective dose via injection, an implant (such as an shunt or stent), an eye drop, an emulsion, a suspension, an ointment, a nanomicelle, a nanoparticle, a nanosuspension, a liposome, an in-situ gel, a contact lens, a reservoir inside or outside the eye, or a microneedle.

In some examples, the inflammatory ophthalmic condition or disease comprises one or more of glaucoma, angiogenesis, leaking blood vessel, protein deposition, fibrosis, extracellular matrix (ECM) deposition, thrombin generation, clot formation, or fibrin formation.

In some examples, the site of the inflammatory ophthalmic condition or disease is selected from the group consisting of an eyelid, a meibomian gland, a trabecular meshwork, Schlemm’s canal, a cornea, a sclera, a vitreous, a choroid, a uvea, and a retina.

In some examples, the inflammatory ophthalmic condition or disease is selected from the group consisting of wet age-related macular degeneration (AMD), dry AMD, diabetic retinopathy, glaucoma, uveitis, cataracts, conjunctivitis, and dry eye disease.

In some examples, the method comprises diagnosing the patient as having the inflammatory ophthalmic condition or disease prior to delivering the therapeutically effective dose of the therapeutic composition.

In some examples, the direct factor Xa inhibitor is selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide(LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), eribaxaban (PD 0348292), 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid(PD0313052), (S)-3-(7-carbamimidoylnaphthalen-2-yl)-2-(4-(((S)-1-(1-iminoethyl)pyrrolidin-3-yl)oxy)phenyl)propanoic acid hydrochloride pentahydrate(DX9065a), letaxaban (TAK-442), GW813893, JTV-803, KFA-144, DPC-423, RPR-209685, MCM-09, or antistasin. For example, the direct factor Xa inhibitor may comprise rivaroxaban, or a salt, isomer, solvate, analog, derivative, metabolite, or prodrugs thereof. Alternatively, or in combination, the direct factor Xa inhibitor may comprise apixaban,

In some examples, an IC₅₀ of the direct factor Xa inhibitor is within a range of about 0.01 nM to about 1000 nM. For example, the IC₅₀ of the direct factor Xa inhibitor is within a range of about 0.1 nM to about 1000 nM, about 1 nM to about 1000 nM, or about 10 nM to about 1000 nM.

In some examples, the therapeutically effective dose of the direct factor Xa inhibitor is within a range of about 1 microgram to 10 mg, more preferably is within a range of about 50 micrograms to about 10 mg. For example, the therapeutically effective dose of the direct factor Xa inhibitor may be within a range of about 0.1 mg to about 10 mg or about 1 mg to about 5 mg.

In some examples, the therapeutically effective dose is sufficient to generate a tissue concentration of the direct factor Xa inhibitor of about 0.001 ng/g tissue to about 100 mg/g tissue, preferably, the therapeutically effective dose is sufficient to generate a tissue concentration of the direct factor Xa inhibitor of about 0.01 ng/g tissue to about 100 mg/g tissue, more preferably, the therapeutically effective dose is sufficient to generate a tissue concentration of the direct factor Xa inhibitor of about 0.1 ng/g tissue to about 100 mg/g tissue . For example, the therapeutically effective dose may be sufficient to generate a tissue concentration of the direct factor Xa inhibitor of about 0.2 ng/g tissue to about 100 mg/g tissue, about 0.5 ng/g tissue to about 100 mg/g tissue, about 1 ng/g tissue to about 100 mg/g tissue, about 10 ng/g tissue to about 100 mg/g tissue, or about 100 ng/g tissue to about 100 mg/g tissue; in about 1 day, 30 days, 60 days, 90 days, or 120 days after introducing the therapeutically effective dose. In some examples, the therapeutically effective dose is sufficient to generate a blood concentration of the direct factor Xa inhibitor which is less than about 200 ng/ml, 100 ng/ml, 50 ng/ml 25 ng/ml, or 10 ng/ml. In some examples, the therapeutically effective dose is sufficient to generate a blood concentration of the direct factor Xa inhibitor which is less than a systemic therapeutic concentration of the direct factor Xa inhibitor for any systemic indication. In some examples, the therapeutically effective dose is sufficient to generate a blood concentration of the direct factor Xa inhibitor which is smaller than a median maximum serum concentration (Cₘₐₓ) of the direct factor Xa inhibitor generated by systemic delivery of the direct factor Xa inhibitor to achieve the same tissue concentration at the site of the inflammatory ophthalmic condition or disease. In some examples, the therapeutically effective dose is sufficient to generate a blood concentration of the direct factor Xa inhibitor which does not exceed a median maximum serum concentration (Cₘₐₓ) of the direct factor Xa inhibitor generated by systemic delivery of the direct factor Xa inhibitor to achieve the same tissue concentration at the site of the inflammatory ophthalmic condition or disease for more than about 6 hours to about 3 days.

In some examples, the therapeutically effective dose is sufficient to maintain a tissue concentration of the direct factor Xa inhibitor of about 0.1 ng/g tissue to about 100 mg/g tissue for about 1 day to about 120 days, for about 1 day to about 1 year, 30 days to about 1 year, 3 months to about 1 year, or 6 months to about 1 year.

In one aspect, a method of treating an inflammatory ophthalmic condition or disease in a patient is provided. The method comprises providing a therapeutic composition comprising a direct factor IIa inhibitor; and delivering a therapeutically effective dose of the therapeutic composition to a site of the inflammatory ophthalmic condition or disease in the patient’s eye.

In some examples, the therapeutically effective dose of the therapeutic composition is effective to suppress or prevent initiation, progression, or relapses of disease, including the progression of established disease.

In some examples, delivering the therapeutically effective dose of the therapeutic composition comprises administering the therapeutically effective dose via injection, an implant (such as an ocular shunt or stent), an eye drop, an emulsion, a suspension, an ointment, a nanomicelle, a nanoparticle, a nanosuspension, a liposome, an in-situ gel, a contact lens, or a microneedle.

In some examples, the inflammatory ophthalmic condition or disease comprises one or more of glaucoma, angiogenesis, leaking blood vessel, protein deposition, fibrosis, extracellular matrix (ECM) deposition, thrombin generation, clot formation, or fibrin formation.

In some examples, the site of the inflammatory ophthalmic condition or disease is selected from the group consisting of an eyelid, a meibomian gland, a trabecular meshwork, Schlemm’s canal, a cornea, a sclera, a vitreous, a choroid, a uvea, and a retina.

In some examples, the inflammatory ophthalmic condition or disease is selected from the group consisting of wet age-related macular degeneration (AMD), dry AMD, diabetic retinopathy, glaucoma, uveitis, cataracts, conjunctivitis, and dry eye disease.

In some examples, the method comprises diagnosing the patient as having the inflammatory ophthalmic condition or disease prior to delivering the therapeutically effective dose of the therapeutic composition.

In some examples, the factor IIa therapeutic composition is selected from the group consisting of argatroban, dabigatran, ximelagatran, melagatran, efegatran, inogatran, atecegatran metoxil (AZD-0837), hirudin, hirudin analogs, bivalirudin, desirudin, or lepirudin. In some examples, the direct factor IIa inhibitor comprises argatroban or a salt, isomer, solvate, analog, derivative, metabolite, or prodrugs thereof.

In some examples, an IC₅₀ of the direct factor Xa inhibitor is within a range of about 0.01 nM to about 1000 nM. For example, the IC₅₀ of the direct factor Xa inhibitor is within a range of about 0.1 nM to about 1000 nM, about 1 nM to about 1000 nM, or about 10 nM to about 1000 nM.

In some examples, the therapeutically effective dose of the direct factor Xa inhibitor is within a range of about 1 microgram to 10 mg, more preferably is within a range of about 50 micrograms to about 10 mg. For example, the therapeutically effective dose of the direct factor Xa inhibitor may be within a range of about 0.1 mg to about 10 mg or about 1 mg to about 5 mg.

In some examples, the therapeutically effective dose is sufficient to generate a tissue concentration of the direct factor IIa inhibitor of about 0.001 ng/g tissue to about 100 mg/g tissue, preferably, the therapeutically effective dose is sufficient to generate a tissue concentration of the direct factor IIa inhibitor of about 0.01 ng/g tissue to about 100 mg/g tissue, more preferably, the therapeutically effective dose is sufficient to generate a tissue concentration of the direct factor IIa inhibitor of about 0.1 ng/g tissue to about 100 mg/g tissue . For example, the therapeutically effective dose may be sufficient to generate a tissue concentration of the direct factor IIa inhibitor of about 0.2 ng/g tissue to about 100 mg/g tissue, about 0.5 ng/g tissue to about 100 mg/g tissue, about 1 ng/g tissue to about 100 mg/g tissue, about 10 ng/g tissue to about 100 mg/g tissue, or about 100 ng/g tissue to about 100 mg/g tissue; in about 1 day, 30 days, 60 days, 90 days, or 120 days after introducing the therapeutically effective dose. In some examples, the therapeutically effective dose is sufficient to generate a blood concentration of the direct factor IIa inhibitor which is less than about 200 ng/ml, 100 ng/ml, 50 ng/ml 25 ng/ml, or 10 ng/ml. In some examples, the therapeutically effective dose is sufficient to generate a blood concentration of the direct factor IIa inhibitor which is less than a systemic therapeutic concentration of the direct factor IIa inhibitor for any systemic indication. In some examples, the therapeutically effective dose is sufficient to generate a blood concentration of the direct factor IIa inhibitor which is smaller than a median maximum serum concentration (Cₘₐₓ) of the direct factor IIa inhibitor generated by systemic delivery of the direct factor IIa inhibitor to achieve the same tissue concentration at the site of the inflammatory ophthalmic condition or disease. In some examples, the therapeutically effective dose is sufficient to generate a blood concentration of the direct factor IIa inhibitor which does not exceed a median maximum serum concentration (Cₘₐₓ) of the direct factor IIa inhibitor generated by systemic delivery of the direct factor IIa inhibitor to achieve the same tissue concentration at the site of the inflammatory ophthalmic condition or disease for more than about 6 hours to about 3 days.

In some examples, the therapeutically effective dose is sufficient to maintain a tissue concentration of the direct factor IIa inhibitor of about 0.1 ng/g tissue to about 100 mg/g tissue for about 1 day to about 120 days, for about 1 day to about 1 year, 30 days to about 1 year, 3 months to about 1 year, or 6 months to about 1 year.

In some examples, the therapeutic composition comprises at least two therapeutic active substances comprising a direct factor Xa inhibitor and a direct factor IIa inhibitor.

In some examples, the therapeutic composition comprising a factor Xa inhibitor further comprises at least one additional therapeutically active substance. In some examples, the at least one additional therapeutically active substance comprises a direct factor IIa inhibitor selected from the group consisting of argatroban, dabigatran, ximelagatran, melagatran, efegatran, inogatran, atecegatran metoxil (AZD-0837), hirudin, hirudin analogs, bivalirudin, desirudin, or lepirudin. In some examples, the direct factor IIa inhibitor comprises argatroban. or a salt, isomer, solvate, analog, derivative, metabolite, or prodrugs thereof. In some examples, the direct factor Xa inhibitor comprises apixaban and the direct factor IIa inhibitor comprises argatroban. In some examples, the therapeutically effective dose of the direct factor IIa inhibitor is within a range of about 50 micrograms to about 10 mg. In some examples, the therapeutically effective dose is sufficient to generate a tissue concentration of the direct factor IIa inhibitor of about 0.1 ng/g tissue to about 100 mg/g tissue. For example, the therapeutically effective dose may be sufficient to generate a tissue concentration of the direct factor IIa inhibitor of about 0.2 ng/g tissue to about 100 mg/g tissue, about 0.5 ng/g tissue to about 100 mg/g tissue, about 1 ng/g tissue to about 100 mg/g tissue, about 10 ng/g tissue to about 100 mg/g tissue, or about 100 ng/g tissue to about 100 mg/g tissue. In some examples, the therapeutically effective dose is sufficient to generate a blood concentration of the direct factor IIa inhibitor which is less than about 200 ng/ml, 100 ng/ml, 50 ng/ml 25 ng/ml, or 10 ng/ml. In some examples, the therapeutically effective dose is sufficient to generate a blood concentration of the direct factor IIa inhibitor which is smaller than a median maximum serum concentration (Cₘₐₓ) of the direct factor IIa inhibitor generated by systemic delivery of the direct factor IIa inhibitor to achieve the same tissue concentration at the site of the inflammatory ophthalmic condition or disease. In some examples, the therapeutically effective dose is sufficient to generate a blood concentration of the direct factor IIa inhibitor which does not exceed a median maximum serum concentration (Cₘₐₓ) of the direct factor IIa inhibitor generated by systemic delivery of the direct factor IIa inhibitor to achieve the same tissue concentration at the site of the inflammatory ophthalmic condition or disease for more than about 6 hours to about 3 days. IN some examples, the therapeutically effective dose is sufficient to maintain a tissue concentration of the direct factor IIa inhibitor of about 0.1 ng/g tissue to about 100 mg/g tissue for about 1 day to about 1 year, 30 days to about 1 year, 3 months to about 1 year, or 6 months to about 1 year. In some examples, the weight compositional ratio of the direct factor Xa inhibitor to the direct factor IIa inhibitor in the therapeutic composition is within a range of about 3:1 to about 1:3. For example, the weight compositional ratio of the direct factor Xa inhibitor to the direct factor IIa inhibitor in the therapeutic composition may be about 1:1.

In some examples, the therapeutic composition comprises one or more anticoagulant agents that has an IC50 to inhibit factor Xa and factor II at a dose ranging from 0.0001 nM to 1000 nM, preferably at a dose ranging from 0.0001 nM to 100 nM, more preferably at a dose ranging from 0.0001 nM to 10 nM, and most preferably at a dose ranging from 0.0001 nM to 1 nM.

In some examples, the therapeutic composition comprising a factor Xa inhibitor, a factor IIa inhibitor, or a combination of a factor Xa inhibitor and a factor IIa inhibitor, administered in combination with one or more additional pharmaceutical agents. In some examples, the one or more additional pharmaceutical agents comprises one or more anti-platelet agent, anti-fibrotic agents, anti-inflammatory agents, anti-proliferative agents, anti-glaucoma agents, immune suppressive agents, anti-diabetic agents, anti-viral agents, anti-angiogenic agents, anti-VEGF agents, serine protease, complement activation inhibitor, anti-histamine agents, or combinations thereof.

In some examples, the anti-fibrotic agent comprises pirfenidone and is present in the therapeutic composition at a concentration sufficient to generate a blood concentration of pirfenidone of less than about 20 µg/ml, 12 µg/ml, 7 µg/ml, or 6.5 µg/ml. In some examples, the anti-fibrotic agent comprises pirfenidone and is present in the therapeutic composition at a concentration sufficient to generate a tissue concentration of pirfenidone of within a range of about 0.1 ng/g tissue to about 100 mg/g tissue. In some examples, the anti-fibrotic agent comprises pirfenidone and is present in the therapeutic composition at a concentration sufficient to generate a tissue concentration of pirfenidone of within a range of about 1 ng/g tissue to about 100 mg/g tissue. In some examples, the anti-fibrotic agent comprises pirfenidone and is present in the therapeutic composition at a concentration sufficient to generate a tissue concentration of pirfenidone of within a range of about 10 ng/g tissue to about 100 mg/g tissue. In some examples, the therapeutically effective dose is sufficient to maintain a tissue concentration of pirfenidone of about 0.1 ng/g tissue to about 100 mg/g tissue for about 1 day to about 1 year, 30 days to about 1 year, 3 months to about 1 year, or 6 months to about 1 year.

In some examples, the anti-fibrotic agent comprises nintedanib and is present in the therapeutic composition at a concentration sufficient to generate a blood concentration of nintedanib of less than about 100 ng/ml, 50 ng/ml, 40 ng/ml, or 30 ng/ml. In some examples, the anti-fibrotic agent comprises nintedanib and is present in the therapeutic composition at a concentration sufficient to generate a tissue concentration of nintedanib of within a range of about 0.1 ng/g tissue to about 100 mg/g tissue. In some examples, the anti-fibrotic agent comprises nintedanib and is present in the therapeutic composition at a concentration sufficient to generate a tissue concentration of nintedanib of within a range of about 1 ng/g tissue to about 100 mg/g tissue. In some examples, the anti-fibrotic agent comprises nintedanib and is present in the therapeutic composition at a concentration sufficient to generate a tissue concentration of nintedanib of within a range of about 10 ng/mg tissue to about 100 mg/g tissue. In some examples, the therapeutically effective dose is sufficient to maintain a tissue concentration of nintedanib of about 0.1 ng/g tissue to about 100 mg/g tissue for about 1 day to about 1 year, 30 days to about 1 year, 3 months to about 1 year, or 6 months to about 1 year. In some examples, the anti-fibrotic agent comprises nintedanib and the therapeutically effective dose comprises nintedanib within a range of about 0.1 mg to about 5 mg.

In some examples, the anti-viral agent or anti-diabetic agent comprises metformin or its salt and is present in the therapeutic composition at a concentration sufficient to generate a blood concentration of metformin or its salt of less than about 1800 µg/ml, 900 µg/ml, 500 µg/ml, 50 µg/ml, 10 µg/ml, 1.3 µg/ml, 1 µg/ml, or 0.8 µg/ml. In some examples, the anti-viral agent or anti-diabetic agent comprises metformin or its salt and is present in the therapeutic composition at a concentration sufficient to generate a tissue concentration of metformin or its salt of within a range of about 0.1 ng/g tissue to about 100 mg/g tissue. In some examples, the anti-viral agent or anti-diabetic agent comprises metformin or its salt and is present in the therapeutic composition at a concentration sufficient to generate a tissue concentration of metformin or its salt of within a range of about 1 ng/g tissue to about 100 mg/g tissue. In some examples, the therapeutically effective dose is sufficient to maintain a tissue concentration of metformin or its salt of about 0.1 ng/mg tissue to about 100 mg/g tissue for about 1 day to about 1 year, 30 days to about 1 year, 3 months to about 1 year, or 6 months to about 1 year.

In some examples, the anti-angiogenic agent comprises an anti-VEGF agent. In some examples, the anti-VEGF agent comprises ranibizumab, aflibercept, pegaptanib sodium, bevacizumab, brolucizumab, lapatinib, sorafenib, axitinib, pazopanib, sunitinib, cabozantinib, lenvatinib, ponatinib, ramucirumab, regorafenib, vandetanib, cediranib, thiazolidinediones, abicipar pegol, faricimab, conbercept, or RGX-314, or other direct or indirect anti-VEGF agents.

In some examples, the anti-VEGF agent comprises ranibizumab and is present in the therapeutic composition at a concentration within a range of about 6 mg/ml to about 40 mg/ml. In some examples, the anti-VEGF agent comprises ranibizumab and the therapeutically effective dose comprises ranibizumab within a range of about 0.3 mg to about 2 mg. In some examples, the anti-VEGF agent comprises ranibizumab and is present in the therapeutic composition at a concentration sufficient to generate a tissue concentration of ranibizumab of within a range of about 0.1 ng/g tissue to about 100 mg/g tissue or about 1 ng/g tissue to about 100 mg/g tissue. In some examples, the therapeutically effective dose is sufficient to maintain a tissue concentration of ranibizumab within a range of about 0.1 ng/g tissue to about 100 mg/g tissue for about 1 day to about 1 year, 30 days to about 1 year, 3 months to about 1 year, or 6 months to about 1 year.

In some examples, the anti-VEGF agent comprises aflibercept and is present in the therapeutic composition at a concentration within a range of about 10 mg/ml to about 100 mg/ml. In some examples, the anti-VEGF agent comprises aflibercept and the therapeutically effective dose comprises aflibercept within a range of about 0.5 mg to about 5 mg. In some examples, the anti-VEGF agent comprises aflibercept and is present in the therapeutic composition at a concentration sufficient to generate a tissue concentration of aflibercept of within a range of about 0.1 ng/g tissue to about 100 mg/g tissue or about 1 ng/g tissue to about 100 mg/g tissue. In some examples, the therapeutically effective dose is sufficient to maintain a tissue concentration of aflibercept within a range of about 0.1 ng/g tissue to about 100 mg/g tissue for about 1 day to about 1 year, 30 days to about 1 year, 3 months to about 1 year, or 6 months to about 1 year.

In some examples, the anti-VEGF agent comprises pegaptanib and is present in the therapeutic composition at a concentration within a range of about 1 mg/ml to about 10 mg/ml. In some examples, the anti-VEGF agent comprises pegaptanib and the therapeutically effective dose comprises pegaptanib within a range of about 0.1 mg to about 1 mg. In some examples, the anti-VEGF agent comprises pegaptanib and is present in the therapeutic composition at a concentration sufficient to generate a tissue concentration of pegaptanib of within a range of about 0.1 ng/g tissue to about 100 mg/g tissue or about 1 ng/g tissue to about 100 mg/g tissue. In some examples, the therapeutically effective dose is sufficient to maintain a tissue concentration of pegaptanib within a range of about 0.1 ng/g tissue to about 100 mg/g tissue for about 1 day to about 1 year, 30 days to about 1 year, 3 months to about 1 year, or 6 months to about 1 year.

In some examples, the anti-proliferative agent comprises an mTOR inhibitor. In some examples, the mTOR inhibitor comprises rapamycin, everolimus, biolimus, temsirolimus, ridaforolimus, zotarolimus, myolimus, or novolimus, or analogues, metabolites, or salts thereof.

In some examples, the anti-proliferative agent comprises sirolimus and is present in the therapeutic composition at a concentration within a range of 1 mg/ml to about 250 mg/ml. In some examples, the anti-proliferative agent comprises sirolimus and the therapeutically effective dose comprises sirolimus within a range of about 50 µg to about 5 mg, about 0.1 mg to about 5 mg, or about 0.5 mg to about 3 mg. In some examples, the anti-proliferative agent comprises sirolimus and is present in the therapeutic composition at a concentration sufficient to generate a tissue concentration of sirolimus of within a range of about 0.1 ng/g tissue to about 100 mg/g tissue. In some examples, the anti-proliferative agent comprises sirolimus and is present in the therapeutic composition at a concentration sufficient to generate a tissue concentration of sirolimus in a sclera of the eye of within a range of about 0.1 ng/g tissue to about 100 mg/g tissue within about 1 day. In some examples, the anti-proliferative agent comprises sirolimus and is present in the therapeutic composition at a concentration sufficient to generate a tissue concentration of sirolimus in a sclera of the eye of within a range of about 0.1 ng/g tissue to about 100 mg/g tissue within about 72 days. In some examples, the anti-proliferative agent comprises sirolimus and is present in the therapeutic composition at a concentration sufficient to maintain a tissue concentration of sirolimus of within a range of about 0.1 ng/g tissue to about 100 mg/g tissue for about 1 day to about 1 year, 30 days to about 1 year, 3 months to about 1 year, or 6 months to about 1 year.

In some examples, the anti-proliferative agent comprises novolimus and is present in the therapeutic composition at a concentration within a range of 1 mg/ml to about 250 mg/ml. In some examples, the anti-proliferative agent comprises novolimus and the therapeutically effective dose comprises novolimus within a range of about 50 µg to about 5 mg, about 0.1 mg to about 5 mg, or about 0.5 mg to about 3 mg. In some examples, the anti-proliferative agent comprises novolimus and is present in the therapeutic composition at a concentration sufficient to generate a tissue concentration of novolimus of within a range of about 0.1 ng/g tissue to about 100 mg/g tissue. In some examples, the anti-proliferative agent comprises novolimus and is present in the therapeutic composition at a concentration sufficient to generate a tissue concentration of novolimus in a sclera of the eye of within a range of about 0.1 ng/g tissue to about 100 mg/g tissue within about 1 day. In some examples, the anti-proliferative agent comprises novolimus and is present in the therapeutic composition at a concentration sufficient to generate a tissue concentration of novolimus in a sclera of the eye of within a range of about 0.1 ng/g tissue to about 100 mg/g tissue within about 72 days. In some examples, the anti-proliferative agent comprises novolimus and is present in the therapeutic composition at a concentration sufficient to maintain a tissue concentration of novolimus of within a range of about 0.1 ng/g tissue to about 100 mg/g tissue for about 1 day to about 1 year, 30 days to about 1 year, 3 months to about 1 year, or 6 months to about 1 year.

In some examples, the anti-proliferative agent comprises myolimus and is present in the therapeutic composition at a concentration within a range of 1 mg/ml to about 250 mg/ml. In some examples, the anti-proliferative agent comprises myolimus and the therapeutically effective dose comprises myolimus within a range of about 50 µg to about 5 mg, about 0.1 mg to about 5 mg, or about 0.5 mg to about 3 mg. In some examples, the anti-proliferative agent comprises myolimus and is present in the therapeutic composition at a concentration sufficient to generate a tissue concentration of myolimus of within a range of about 0.1 ng/g tissue to about 100 mg/g tissue. In some examples, the anti-proliferative agent comprises myolimus and is present in the therapeutic composition at a concentration sufficient to generate a tissue concentration of myolimus in a sclera of the eye of within a range of about 0.1 ng/g tissue to about 100 mg/g tissue within about 1 day. In some examples, the anti-proliferative agent comprises myolimus and is present in the therapeutic composition at a concentration sufficient to generate a tissue concentration of myolimus in a sclera of the eye of within a range of about 0.1 ng/g tissue to about 100 mg/g tissue within about 72 days. In some examples, the anti-proliferative agent comprises myolimus and is present in the therapeutic composition at a concentration sufficient to maintain a tissue concentration of myolimus of within a range of about 0.1 ng/g tissue to about 100 mg/g tissue for about 1 day to about 1 year, 30 days to about 1 year, 3 months to about 1 year, or 6 months to about 1 year.

In some examples, the therapeutically effective dose is sufficient to generate a blood concentration of the anti-proliferative agent which is smaller than a median maximum serum concentration (Cₘₐₓ) of the anti-proliferative agent generated by systemic delivery of the anti-proliferative agent to achieve the same tissue concentration at the site of the inflammatory ophthalmic condition or disease. In some examples, the therapeutically effective dose is sufficient to generate a blood concentration of the anti-proliferative agent which does not exceed a median maximum serum concentration (Cₘₐₓ) of the anti-proliferative agent generated by systemic delivery of the anti-proliferative agent to achieve the same tissue concentration at the site of the inflammatory ophthalmic condition or disease for more than about 6 hours to about 7 days. In some examples, the therapeutically effective dose is sufficient to generate a blood concentration of the anti-proliferative agent which is less than about 10 ng/ml.

In some examples, the serine protease agent comprises tissue plasminogen activator( t-PA), urokinase-type plasminogen activator (u-PA), Streptokinase or plasmin and is present in the therapeutic composition at a concentration within a range of 1 mg/ml to about 250 mg/ml. In some examples, the serine protease agent comprises t-PA and the therapeutically effective dose comprises a range of about 50 µg to about 5 mg, about 25 µg to about 5 mg, or about 0.3 µg to about 3 mg.

In some examples, the thrombolytic drugs comprise complement inhibitors target the modulation of complement proteins C3, C5, factor B, factor D, and properdin. Complement activation inhibitors include but not limit to C3 inhibitor such as APL-2,Compstatin and AMY106, Anti-factor D such as Lampalizumab, Anti-Properdin such as CLG561, Anti-C5 such as Eculizumab, Ravulizumab, ARC1905, and LFG316, CD59 such as AAVCAGsCD59, analogues and the like. In some examples, the thrombolytic drugs comprise complement activation inhibitors and the therapeutically effective dose comprises a range of about 20 µg to about 2000 mg, about 10 µg to about 1500 mg, or about 5 µg to about 1200 mg.

In some examples, the therapeutic composition comprises one or more of a pharmaceutically acceptable carrier, a diluent, an adjuvant, an excipient, a vehicle, a preserving agent, a filler, a polymer, a disintegrating agent, a glidant, a wetting agent, an emulsifying agent, a suspending agent, a sweetening agent, a flavoring agent, a perfuming agent, a lubricating agent, an acidifying agent, and a dispensing agent.

In some examples, the therapeutically effective dose of the therapeutic composition is effective to inhibit thrombosis, inhibit clot formation, or inhibit fibrin formation in the patient’s eye.

In another aspect, a therapeutic composition for inhibiting an inflammatory ophthalmic condition or disease in a patient is provided. The composition comprises a direct factor Xa inhibitor formulated for delivery to an eye of the patient by any one of injection, implant (such as an ocular shunt or stent), eye drop, emulsion, suspension, ointment, nanocarrier, nanomicelle, nanoparticle, nanosuspension, liposome, in-situ gel, contact lens, reservoir (implanted insider or outside the eye) or microneedle.

In some examples, delivery of a therapeutically effective dose of the therapeutic composition is effective to suppress or prevent initiation, progression, or relapses of disease, including the progression of established disease.

In some examples, the inflammatory ophthalmic condition or disease comprises one or more of angiogenesis, leaking blood vessel, protein deposition, fibrosis, extracellular matrix (ECM) deposition, thrombin generation, clot formation, glaucoma, or fibrin formation.

In some examples, a site of the inflammatory ophthalmic condition or disease is selected from the group consisting of an eyelid, a meibomian gland, a trabecular meshwork, Schlemm’s canal, a cornea, a sclera, a vitreous, a choroid, a uvea, and a retina.

In some examples, the inflammatory ophthalmic condition or disease is selected from the group consisting of wet age-related macular degeneration (AMD), dry AMD, diabetic retinopathy, glaucoma, uveitis, cataracts, conjunctivitis, glaucoma, and dry eye disease.

In some examples, the direct factor Xa inhibitor is selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide(LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid(PD0313052), (S)-3-(7-carbamimidoylnaphthalen-2-yl)-2-(4-(((S)-1-(1-iminoethyl)pyrrolidin-3-yl)oxy)phenyl)propanoic acid hydrochloride pentahydrate(DX9065a), letaxaban (TAK-442), GW813893, JTV-803, KFA-144, DPC-423, RPR-209685, MCM-09, or antistasin. For example, the direct factor Xa inhibitor may comprise rivaroxaban, or a salt, isomer, solvate, analog, derivative, metabolite, or prodrugs thereof. Alternatively, or in combination, the direct factor Xa inhibitor may comprise apixaban, or a salt, isomer, solvate, analog, derivative, metabolite, or prodrugs thereof.

In some examples, an IC₅₀ of the direct factor Xa inhibitor is within a range of about 0.01 nM to about 1000 nM. For example, the IC₅₀ of the direct factor Xa inhibitor is within a range of about 0.1 nM to about 1000 nM, about 1 nM to about 1000 nM, or about 10 nM to about 1000 nM.

In some examples, a therapeutically effective dose of the direct factor Xa inhibitor is within a range of about 50 micrograms to about 10 mg. For example, the therapeutically effective dose of the direct factor Xa inhibitor may be within a range of about 0.1 mg to about 10 mg or about 1 mg to about 5 mg.

In some examples, a therapeutically effective dose of the direct factor Xa inhibitor is sufficient to generate a tissue concentration of the direct factor Xa inhibitor of about 0.1 ng/g tissue to about 100 mg/g tissue. For example, the therapeutically effective dose may be sufficient to generate a tissue concentration of the direct factor Xa inhibitor of about 0.2 ng/g tissue to about 100 mg/g tissue, about 0.5 ng/g tissue to about 100 mg/g tissue, about 1 ng/g tissue to about 100 mg/g tissue, about 10 ng/g tissue to about 100 mg/g tissue, or about 100 ng/g tissue to about 100 mg/g tissue. In some examples, the therapeutically effective dose is sufficient to generate a blood concentration of the direct factor Xa inhibitor which is less than about 200 ng/ml, 100 ng/ml, 50 ng/ml 25 ng/ml, or 10 ng/ml. In some examples, the therapeutically effective dose is sufficient to generate a blood concentration of the direct factor Xa inhibitor which is less than a systemic therapeutic concentration of the direct factor Xa inhibitor for any systemic indication. In some examples, the therapeutically effective dose is sufficient to generate a blood concentration of the direct factor Xa inhibitor which is smaller than a median maximum serum concentration (Cₘₐₓ) of the direct factor Xa inhibitor generated by systemic delivery of the direct factor Xa inhibitor to achieve the same tissue concentration at the site of the inflammatory ophthalmic condition or disease. In some examples, the therapeutically effective dose is sufficient to generate a blood concentration of the direct factor Xa inhibitor which does not exceed a median maximum serum concentration (Cₘₐₓ) of the direct factor Xa inhibitor generated by systemic delivery of the direct factor Xa inhibitor to achieve the same tissue concentration at the site of the inflammatory ophthalmic condition or disease for more than about 6 hours to about 3 days.

In some examples, a therapeutically effective dose of the direct factor Xa inhibitor is sufficient to maintain a tissue concentration of the direct factor Xa inhibitor of about 0.1 ng/g tissue to about 100 mg/g tissue for about 1 day to about 1 year, 30 days to about 1 year, 3 months to about 1 year, or 6 months to about 1 year.

In some examples, the therapeutic composition further comprises at least one additional therapeutically active substance. In some examples, the at least one additional therapeutically active substance comprises a direct factor IIa inhibitor selected from the group consisting of argatroban, dabigatran, ximelagatran, melagatran, efegatran, inogatran, atecegatran metoxil (AZD-0837), hirudin, hirudin analogs, bivalirudin, desirudin, or lepirudin. In some examples, the direct factor IIa inhibitor comprises argatroban. or a salt, isomer, solvate, analog, derivative, metabolite, or prodrugs thereof. In some examples, the direct factor Xa inhibitor comprises apixaban and the direct factor IIa inhibitor comprises argatroban. In some examples, a therapeutically effective dose of the direct factor IIa inhibitor is within a range of about 50 micrograms to about 10 mg. In some examples, the therapeutically effective dose is sufficient to generate a tissue concentration of the direct factor IIa inhibitor of about 0.1 ng/g tissue to about 100 mg/g tissue. For example, the therapeutically effective dose may be sufficient to generate a tissue concentration of the direct factor IIa inhibitor of about 0.2 ng/g tissue to about 100 mg/g tissue, about 0.5 ng/g tissue to about 100 mg/g tissue, about 1 ng/g tissue to about 100 mg/g tissue, about 10 ng/g tissue to about 100 mg/g tissue, or about 100 ng/g tissue to about 100 mg/g tissue. In some examples, the therapeutically effective dose is sufficient to generate a blood concentration of the direct factor IIa inhibitor which is less than about 200 ng/ml, 100 ng/ml, 50 ng/ml 25 ng/ml, or 10 ng/ml. In some examples, the therapeutically effective dose is sufficient to generate a blood concentration of the direct factor IIa inhibitor which is smaller than a median maximum serum concentration (Cₘₐₓ) of the direct factor IIa inhibitor generated by systemic delivery of the direct factor IIa inhibitor to achieve the same tissue concentration at the site of the inflammatory ophthalmic condition or disease. In some examples, the therapeutically effective dose is sufficient to generate a blood concentration of the direct factor IIa inhibitor which does not exceed a median maximum serum concentration (Cₘₐₓ) of the direct factor IIa inhibitor generated by systemic delivery of the direct factor IIa inhibitor to achieve the same tissue concentration at the site of the inflammatory ophthalmic condition or disease for more than about 6 hours to about 3 days. IN some examples, the therapeutically effective dose is sufficient to maintain a tissue concentration of the direct factor IIa inhibitor of about 0.1 ng/g tissue to about 100 mg/g tissue for about 1 day to about 1 year, 30 days to about 1 year, 3 months to about 1 year, or 6 months to about 1 year. In some examples, the weight compositional ratio of the direct factor Xa inhibitor to the direct factor IIa inhibitor in the therapeutic composition is within a range of about 3:1 to about 1:3. For example, the weight compositional ratio of the direct factor Xa inhibitor to the direct factor IIa inhibitor in the therapeutic composition may be about 1:1.

In some examples, the therapeutic composition is administered in combination with one or more additional pharmaceutical agents. In some examples, the one or more additional pharmaceutical agents comprises one or more anti-fibrotic agents, anti-inflammatory agents, anti-histamine agents, anti-proliferative agents, anti-diabetic agents, anti-viral agents, metformin, anti-angiogenic agents, or combinations thereof.

In some examples, the anti-fibrotic agent comprises pirfenidone and is present in the therapeutic composition at a concentration sufficient to generate a blood concentration of pirfenidone of less than about 20 µg/ml, 12 µg/ml, 7 µg/ml, or 6.5 µg/ml. In some examples, the anti-fibrotic agent comprises pirfenidone and is present in the therapeutic composition at a concentration sufficient to generate a tissue concentration of pirfenidone of within a range of about 0.1 ng/g tissue to about 100 mg/g tissue. In some examples, the anti-fibrotic agent comprises pirfenidone and is present in the therapeutic composition at a concentration sufficient to generate a tissue concentration of pirfenidone of within a range of about 1 ng/g tissue to about 100 mg/g tissue. In some examples, the anti-fibrotic agent comprises pirfenidone and is present in the therapeutic composition at a concentration sufficient to generate a tissue concentration of pirfenidone of within a range of about 10 ng/g tissue to about 100 mg/g tissue. In some examples, the therapeutically effective dose is sufficient to maintain a tissue concentration of pirfenidone of about 0.1 ng/g tissue to about 100 mg/g tissue for about 1 day to about 1 year, 30 days to about 1 year, 3 months to about 1 year, or 6 months to about 1 year.

In some examples, the anti-fibrotic agent comprises nintedanib and is present in the therapeutic composition at a concentration sufficient to generate a blood concentration of nintedanib of less than about 100 ng/ml, 50 ng/ml, 40 ng/ml, or 30 ng/ml. In some examples, the anti-fibrotic agent comprises nintedanib and is present in the therapeutic composition at a concentration sufficient to generate a tissue concentration of nintedanib of within a range of about 0.1 ng/g tissue to about 100 mg/g tissue. In some examples, the anti-fibrotic agent comprises nintedanib and is present in the therapeutic composition at a concentration sufficient to generate a tissue concentration of nintedanib of within a range of about 1 ng/g tissue to about 100 mg/g tissue. In some examples, the anti-fibrotic agent comprises nintedanib and is present in the therapeutic composition at a concentration sufficient to generate a tissue concentration of nintedanib of within a range of about 10 ng/mg tissue to about 100 mg/g tissue. In some examples, the therapeutically effective dose is sufficient to maintain a tissue concentration of nintedanib of about 0.1 ng/g tissue to about 100 mg/g tissue for about 1 day to about 1 year, 30 days to about 1 year, 3 months to about 1 year, or 6 months to about 1 year. In some examples, the anti-fibrotic agent comprises nintedanib and the therapeutically effective dose comprises nintedanib within a range of about 0.1 mg to about 5 mg.

In some examples, the anti-viral agent or anti-diabetic agent comprises metformin or its salt and is present in the therapeutic composition at a concentration sufficient to generate a blood concentration of metformin or its salt of less than about 1800 µg/ml, 900 µg/ml, 500 µg/ml, 50 µg/ml, 10 µg/ml, 1.3 µg/ml, 1 µg/ml, or 0.8 µg/ml. In some examples, the anti-viral agent or anti-diabetic agent comprises metformin or its salt and is present in the therapeutic composition at a concentration sufficient to generate a tissue concentration of metformin or its salt of within a range of about 0.1 ng/g tissue to about 100 mg/g tissue. In some examples, the anti-viral agent or anti-diabetic agent comprises metformin or its salt and is present in the therapeutic composition at a concentration sufficient to generate a tissue concentration of metformin or its salt of within a range of about 1 ng/g tissue to about 100 mg/g tissue. In some examples, the therapeutically effective dose is sufficient to maintain a tissue concentration of metformin or its salt of about 0.1 ng/mg tissue to about 100 mg/g tissue for about 1 day to about 1 year, 30 days to about 1 year, 3 months to about 1 year, or 6 months to about 1 year.

In some examples, the anti-angiogenic agent comprises an anti-VEGF agent. In some examples, the anti-VEGF agent comprises ranibizumab, aflibercept, pegaptanib sodium, bevacizumab, brolucizumab, lapatinib, sorafenib, axitinib, pazopanib, sunitinib, cabozantinib, lenvatinib, ponatinib, ramucirumab, regorafenib, vandetanib, cediranib, thiazolidinediones or RGX-314.

In some examples, the anti-VEGF agent comprises ranibizumab and is present in the therapeutic composition at a concentration within a range of about 6 mg/ml to about 40 mg/ml. In some examples, the anti-VEGF agent comprises ranibizumab and the therapeutically effective dose comprises ranibizumab within a range of about 0.3 mg to about 2 mg. In some examples, the anti-VEGF agent comprises ranibizumab and is present in the therapeutic composition at a concentration sufficient to generate a tissue concentration of ranibizumab of within a range of about 0.1 ng/g tissue to about 100 mg/g tissue or about 1 ng/g tissue to about 100 mg/g tissue. In some examples, the therapeutically effective dose is sufficient to maintain a tissue concentration of ranibizumab within a range of about 0.1 ng/g tissue to about 100 mg/g tissue for about 1 day to about 1 year, 30 days to about 1 year, 3 months to about 1 year, or 6 months to about 1 year.

In some examples, the anti-VEGF agent comprises brolucizumab and is present in the therapeutic composition at a concentration within a range of about 50 mg/ml to about 500 mg/ml or more. In some examples, the anti-VEGF agent comprises brolucizumab and the therapeutically effective dose comprises brolucizumab within a range of about 0.3 mg to about 30 mg. In some examples, the anti-VEGF agent comprises brolucizumab and is present in the therapeutic composition at a concentration sufficient to generate a tissue concentration of brolucizumab of within a range of about 0.1 ng/g tissue to about 100 mg/g tissue or about 1 ng/g tissue to about 100 mg/g tissue. In some examples, the therapeutically effective dose is sufficient to maintain a tissue concentration of brolucizumab within a range of about 0.1 ng/g tissue to about 100 mg/g tissue for about 1 day to about 1 year, 30 days to about 1 year, 3 months to about 1 year, or 6 months to about 1 year.

In some examples, the anti-VEGF agent comprises aflibercept and is present in the therapeutic composition at a concentration within a range of about 10 mg/ml to about 100 mg/ml. In some examples, the anti-VEGF agent comprises aflibercept and the therapeutically effective dose comprises aflibercept within a range of about 0.5 mg to about 5 mg. In some examples, the anti-VEGF agent comprises aflibercept and is present in the therapeutic composition at a concentration sufficient to generate a tissue concentration of aflibercept of within a range of about 0.1 ng/g tissue to about 100 mg/g tissue or about 1 ng/g tissue to about 100 mg/g tissue. In some examples, the therapeutically effective dose is sufficient to maintain a tissue concentration of aflibercept within a range of about 0.1 ng/g tissue to about 100 mg/g tissue for about 1 day to about 1 year, 30 days to about 1 year, 3 months to about 1 year, or 6 months to about 1 year.

In some examples, the anti-VEGF agent comprises pegaptanib and is present in the therapeutic composition at a concentration within a range of about 1 mg/ml to about 10 mg/ml. In some examples, the anti-VEGF agent comprises pegaptanib and the therapeutically effective dose comprises pegaptanib within a range of about 0.1 mg to about 1 mg. In some examples, the anti-VEGF agent comprises pegaptanib and is present in the therapeutic composition at a concentration sufficient to generate a tissue concentration of pegaptanib of within a range of about 0.1 ng/g tissue to about 100 mg/g tissue or about 1 ng/g tissue to about 100 mg/g tissue. In some examples, the therapeutically effective dose is sufficient to maintain a tissue concentration of pegaptanib within a range of about 0.1 ng/g tissue to about 100 mg/g tissue for about 1 day to about 1 year, 30 days to about 1 year, 3 months to about 1 year, or 6 months to about 1 year.

In some examples, the anti-proliferative agent comprises an mTOR inhibitor. In some examples, the mTOR inhibitor comprises rapamycin, everolimus, biolimus, temsirolimus, ridaforolimus, zotarolimus, myolimus, or novolimus.

In some examples, the anti-proliferative agent comprises sirolimus and is present in the therapeutic composition at a concentration within a range of 1 mg/ml to about 250 mg/ml. In some examples, the anti-proliferative agent comprises sirolimus and the therapeutically effective dose comprises sirolimus within a range of about 50 µg to about 5 mg, about 0.1 mg to about 5 mg, or about 0.5 mg to about 3 mg. In some examples, the anti-proliferative agent comprises sirolimus and is present in the therapeutic composition at a concentration sufficient to generate a tissue concentration of sirolimus of within a range of about 0.1 ng/g tissue to about 100 mg/g tissue. In some examples, the anti-proliferative agent comprises sirolimus and is present in the therapeutic composition at a concentration sufficient to generate a tissue concentration of sirolimus in a sclera of the eye of within a range of about 0.1 ng/g tissue to about 100 mg/g tissue within about 1 day. In some examples, the anti-proliferative agent comprises sirolimus and is present in the therapeutic composition at a concentration sufficient to generate a tissue concentration of sirolimus in a sclera of the eye of within a range of about 0.1 ng/g tissue to about 100 mg/g tissue within about 72 days. In some examples, the anti-proliferative agent comprises sirolimus and is present in the therapeutic composition at a concentration sufficient to maintain a tissue concentration of sirolimus of within a range of about 0.1 ng/g tissue to about 100 mg/g tissue for about 1 day to about 1 year, 30 days to about 1 year, 3 months to about 1 year, or 6 months to about 1 year.

In some examples, the anti-proliferative agent comprises novolimus and is present in the therapeutic composition at a concentration within a range of 1 mg/ml to about 250 mg/ml. In some examples, the anti-proliferative agent comprises novolimus and the therapeutically effective dose comprises novolimus within a range of about 50 µg to about 5 mg, about 0.1 mg to about 5 mg, or about 0.5 mg to about 3 mg. In some examples, the anti-proliferative agent comprises novolimus and is present in the therapeutic composition at a concentration sufficient to generate a tissue concentration of novolimus of within a range of about 0.1 ng/g tissue to about 100 mg/g tissue. In some examples, the anti-proliferative agent comprises novolimus and is present in the therapeutic composition at a concentration sufficient to generate a tissue concentration of novolimus in a sclera of the eye of within a range of about 0.1 ng/g tissue to about 100 mg/g tissue within about 1 day. In some examples, the anti-proliferative agent comprises novolimus and is present in the therapeutic composition at a concentration sufficient to generate a tissue concentration of novolimus in a sclera of the eye of within a range of about 0.1 ng/g tissue to about 100 mg/g tissue within about 72 days. In some examples, the anti-proliferative agent comprises novolimus and is present in the therapeutic composition at a concentration sufficient to maintain a tissue concentration of novolimus of within a range of about 0.1 ng/g tissue to about 100 mg/g tissue for about 1 day to about 1 year, 30 days to about 1 year, 3 months to about 1 year, or 6 months to about 1 year.

In some examples, the anti-proliferative agent comprises myolimus and is present in the therapeutic composition at a concentration within a range of 1 mg/ml to about 250 mg/ml. In some examples, the anti-proliferative agent comprises myolimus and the therapeutically effective dose comprises myolimus within a range of about 50 µg to about 5 mg, about 0.1 mg to about 5 mg, or about 0.5 mg to about 3 mg. In some examples, the anti-proliferative agent comprises myolimus and is present in the therapeutic composition at a concentration sufficient to generate a tissue concentration of myolimus of within a range of about 0.1 ng/g tissue to about 100 mg/g tissue. In some examples, the anti-proliferative agent comprises myolimus and is present in the therapeutic composition at a concentration sufficient to generate a tissue concentration of myolimus in a sclera of the eye of within a range of about 0.1 ng/g tissue to about 100 mg/g tissue within about 1 day. In some examples, the anti-proliferative agent comprises myolimus and is present in the therapeutic composition at a concentration sufficient to generate a tissue concentration of myolimus in a sclera of the eye of within a range of about 0.1 ng/g tissue to about 100 mg/g tissue within about 72 days. In some examples, the anti-proliferative agent comprises myolimus and is present in the therapeutic composition at a concentration sufficient to maintain a tissue concentration of myolimus of within a range of about 0.1 ng/g tissue to about 100 mg/g tissue for about 1 day to about 1 year, 30 days to about 1 year, 3 months to about 1 year, or 6 months to about 1 year.

In some examples, the therapeutically effective dose is sufficient to generate a blood concentration of the anti-proliferative agent which is smaller than a median maximum serum concentration (Cₘₐₓ) of the anti-proliferative agent generated by systemic delivery of the anti-proliferative agent to achieve the same tissue concentration at the site of the inflammatory ophthalmic condition or disease. In some examples, the therapeutically effective dose is sufficient to generate a blood concentration of the anti-proliferative agent which does not exceed a median maximum serum concentration (Cₘₐₓ) of the anti-proliferative agent generated by systemic delivery of the anti-proliferative agent to achieve the same tissue concentration at the site of the inflammatory ophthalmic condition or disease for more than about 6 hours to about 7 days. In some examples, the therapeutically effective dose is sufficient to generate a blood concentration of the anti-proliferative agent which is less than about 10 ng/ml.

In some examples, the therapeutic composition comprises one or more of a pharmaceutically acceptable carrier, a diluent, an adjuvant, an excipient, a vehicle, a preserving agent, a filler, a polymer, a disintegrating agent, a glidant, a wetting agent, an emulsifying agent, a suspending agent, a sweetening agent, a flavoring agent, a perfuming agent, a lubricating agent, an acidifying agent, and a dispensing agent.

In some examples, delivery of a therapeutically effective dose of the therapeutic composition is effective to inhibit thrombosis, inhibit clot formation, or inhibit fibrin formation in the patient’s eye.

In another aspect, a method for inhibiting clot formation or fibrin formation in an eye of a patient is provided. The method comprises providing a therapeutic composition comprising a direct factor Xa inhibitor; and delivering a therapeutically effective dose of the therapeutic composition to a site of the clot formation or fibrin formation in the patient’s eye.

The illustrative examples described are not meant to be limiting. Other examples may be utilized, and other changes may be made, or combined in whole or in part, without departing from the scope of the subject matter presented herein. It will be readily understood that the aspects of the present disclosure, as generally described herein, and illustrated in the figures, and detailed description, and in the examples, can be arranged, substituted, combined, separated, and designed in a wide variety of different configurations, all of which are explicitly contemplated herein.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows a plot of HAoSMC cell proliferation in the presence of rapamycin and varying concentrations of Apixaban, in accordance with examples;

FIG. 1B shows a plot of HAoSMC cell proliferation in the presence of rapamycin and varying concentrations of Argatroban, in accordance with examples;

FIG. 1C shows a plot of HAoSMC cell proliferation in the presence of rapamycin and varying concentrations of Apixaban and Argatroban, in accordance with examples;

FIG. 1D shows a plot of HAoSMC cell proliferation in the presence of difference concentrations of Apixaban, in accordance with examples;

FIG. 1E shows a plot of HAoSMC cell proliferation in the presence of difference concentrations of Argatroban, in accordance with examples;

FIG. 2A shows a plot of activated clotting time (ACT) versus drug concentration, in accordance with examples;

FIG. 2B shows a plot of activated clotting time (ACT) versus drug concentration, in accordance with examples;

FIG. 2C shows a plot of activated clotting time (ACT) versus drug concentration, showing the synergistic effects of Apixaban in combination with Argatroban, in accordance with examples;

FIG. 2D shows a plot of various synergistic effects of drug combination ratios between Apixaban and Argatroban, in accordance with examples;

FIG. 3 shows a reaction scheme of Argatroban with poly N-(2-Hydroxypropyl) methacrylamide, in accordance with examples.

### DETAILED DESCRIPTION OF THE DISCLOSURE

In the following detailed description, reference is made to the accompanying figures, which form a part hereof. In the figures, similar symbols typically identify similar components, unless context dictates otherwise. The illustrative examples described in the detailed description, figures, and claims are not meant to be limiting. Other examples may be utilized, and other changes may be made, or combined in whole or in part, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain examples and examples are disclosed below, inventive subject matter extends beyond the specifically disclosed examples to other alternative examples and/or uses, and to modifications and equivalents thereof. Thus, the scope of the claims appended hereto is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples, however, the order of description should not be construed to imply that these operations are order dependent. Additionally, the structures, systems, and/or devices described herein may be embodied as integrated components or as separate components.

For purposes of comparing various examples, certain aspects and advantages of these examples are described. Not necessarily all such aspects or advantages are achieved by any particular embodiment. Thus, for example, various examples may be carried out in a manner that achieves or optimizes one advantage or group of advantages as taught herein without necessarily achieving other aspects or advantages as may also be taught or suggested herein.

Every embodiment of the present disclosure may optionally be combined with any one or more of the other examples described herein. Every patent literature, and every nonpatent literature, cited herein is incorporated herein by reference in its entirety.

The present disclosure is described in relation to treating ophthalmic disease or conditions (e.g., inflammatory ophthalmic condition or disease) in a patient via ocular delivery of a therapeutic composition. However, one of skill in the art will appreciate that this is not intended to be limiting and the devices and methods disclosed herein may be used in other anatomical areas and in other therapeutic procedures.

As used herein, the term coagulation comprises one or more of thrombin formation, fibrin formation, platelet activation, platelet aggregation, and/or thrombus/clot formation. Coagulation typically arises in response to a body part injury and/or to a foreign body such as a device, and/or an infection from a virus or bacteria, and/or caused by irritants.

As used herein, the term anti-coagulant refers to an agent that inhibits one or more of immune activation or reaction, thrombin formation, fibrin formation, platelet activation, platelet aggregation, thrombus (clot) formation, thrombin dissolution, fibrin dissolution, or thrombus dissolution, thereby inhibiting one or more of inflammation, thrombin, clot, immune activation or reaction, glaucoma, or fibrin..

The terms anti-thrombin, thrombin inhibitor, and thrombin formation inhibitor are used interchangeably herein. Also, the terms anti-fibrin, fibrin inhibitor, and fibrin formation inhibitor are used interchangeably herein.

The terms therapeutically active substance and pharmaceutical agent are used interchangeably herein and refer to any bioactive agent. It will be understood by one of ordinary skill in the art that the devices and methods described herein may be used in combination with one or more additional bioactive agents. Such substances and/or agents optionally include anticoagulants, anti-mitotic agents, anti-proliferative agents, cytostatic agents, anti-migratory agents, anti-fibrotic agents, immunomodulators, immunosuppressants, anti-inflammatory agents, anti-ischemia agents, anti-hypertensive agents, vasodilators, anti-hyperlipidemia agents, antidiabetic agents, metformin, anti-cancer agents, anti-tumor agents, anti-angiogenic agents, antihistamine agents, angiogenic agents, anti-chemokine agents, healing-promoting agents, antiviral agents, anti-bacterial agents, anti-fungal agents, steroids, interferons, and combinations thereof. It is understood that a bioactive agent may exert more than one biological effect.

As used herein, a direct factor Xa inhibitor refers to a direct, selective inhibitor of factor Xa that acts directly on factor Xa without using antithrombin as a mediator. The term “direct factor Xa inhibitor” is used herein interchangeably with the term “factor Xa inhibitor” or “anti-factor Xa”. Direct factor Xa inhibitors inhibit thrombin formation and/or fibrin formation, thereby inhibiting clot formation. Direct factor Xa inhibitors include, but are not limited to, apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide(LY-517717), daraxaban (YM-150), or 2-[(7-carbamimidoylnaphthalen-2-yl)methyl-[4-(1-ethanimidoylpiperidin-4-yl)oxyphenyl]sulfamoyl]acetic acid (YM-466 or YM-60828), and eribaxaban (PD 0348292), or 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid(PD0313052), (S)-3-(7-carbamimidoylnaphthalen-2-yl)-2-(4-(((S)-1-(1-iminoethyl)pyrrolidin-3-yl)oxy)phenyl)propanoic acid hydrochloride pentahydrate(DX9065a), letaxaban (TAK-442), GW813893, JTV-803, KFA-144, DPC-423, RPR-209685, MCM-09, and antistasin, and salt, isomer, solvate, analog, derivative, metabolite, or prodrugs thereof. Preferred direct Xa inhibitors include apixaban and rivaroxaban.

As used herein, a direct factor IIa inhibitor refers to a direct, selective inhibitor of factor IIa (also referred to herein as thrombin) which acts directly on factor IIa/thrombin. The term “direct factor IIa inhibitor” is used herein interchangeably with the term “factor IIa inhibitor” or “anti-factor IIa”. Direct factor IIa inhibitors inhibit thrombin formation and/or fibrin formation, thereby inhibiting clot formation. Direct thrombin/factor IIa inhibitors include, but are not limited to, argatroban, dabigatran, ximelagatran, melagatran, efegatran, inogatran, atecegatran metoxil (AZD-0837), hirudin, hirudin analogs, bivalirudin, desirudin, and lepirudin, and salt, isomer, solvate, analog, derivative, metabolite, or prodrugs thereof. Preferred direct factor IIa inhibitors include argatroban.

As used herein, an anti-fibrotic agent refers to an agent which acts to reduce or eliminate tissue fibrosis. Anti-fibrotic agents include, but are not limited to, nintedanib, pirfenidone, and salt, isomer, solvate, analog, derivative, metabolite, or prodrugs thereof.

As used herein, an anti-proliferative agent refers to anti-proliferative agents, anti-mitotic agents, cytostatic agents and anti-migratory agents which suppress cell growth, proliferation, and/or metabolism. Examples of anti-proliferative agents include without limitation inhibitors of mammalian target of rapamycin (mTOR), rapamycin (also called sirolimus), deuterated rapamycin, rapamycin prodrug TAFA93, 40-O-alkyl-rapamycin derivatives, 40-O-hydroxyalkyl-rapamycin derivatives, everolimus {40-O-(2-hydroxyethyl)-rapamycin}, 40-O-(3-hydroxy)propyl-rapamycin, 40-O-[2-(2-hydroxy)ethoxy]ethyl-rapamycin, 40-O-alkoxyalkyl-rapamycin derivatives, biolimus {40-O-(2-ethoxyethyl)-rapamycin}, 40-O-acyl-rapamycin derivatives, temsirolimus {40-(3-hydroxy-2-hydroxymethyl-2-methylpropanoate)-rapamycin, or CCI-779 (temsirolimus), 40-O-phospho-containing rapamycin derivatives, ridaforolimus (40-dimethylphosphinate-rapamycin, or AP23573 (ridaforolimus, formerly known as deforolimus), 40(R or S)-heterocyclyl- or heteroaryl-containing rapamycin derivatives, zotarolimus {40-epi-(N1-tetrazolyl)-rapamycin, or ABT-578 (zotarolimus), 40-epi-(N2-tetrazolyl)-rapamycin, 32(R or S)-hydroxy-rapamycin, myolimus (32-deoxo-rapamycin), novolimus (16-O-desmethyl-rapamycin), taxanes, paclitaxel, docetaxel, cytochalasins, cytochalasins A through J, latrunculins, and salts, isomers, solvates, analogs (including deuterated analogs), derivatives, metabolites, and prodrugs thereof. The IUPAC numbering system for rapamycin is used herein. Preferred anti-proliferative agents include mTOR inhibitors and/or taxanes, or salts, isomers, solvates, analogs, derivatives, metabolites, or prodrugs thereof.

Table 1 provides non-limiting examples of derivatives of each of rapamycin, everolimus, biolimus, temsirolimus, ridaforolimus, zotarolimus, myolimus and novolimus.

TABLE 1

Derivatives of rapamycin-type compounds
Derivatives of Each of Rapamycin, Everolimus, Biolimus, Temsirolimus, Ridaforolimus, Zotarolimus, Myolimus and Novolimus
N7-oxide
2-hydroxy
3-hydroxy
4-hydroxy
5-hydroxy
6-hydroxy
11-hydroxy
12-hydroxy
13-hydroxy
14-hydroxy
23-hydroxy
24-hydroxy
25-hydroxy
31-hydroxy
35-hydroxy
43-hydroxy (11-hydroxymethyl)
44-hydroxy (17-hydroxymethyl)
45-hydroxy (23-hydroxymethyl)
46-hydroxy (25-hydroxymethyl)
47-hydroxy (29-hydroxymethyl)
48-hydroxy (31-hydroxymethyl)
49-hydroxy (35-hydroxymethyl)
17,18-dihydroxy
19,20-dihydroxy
21,22-dihydroxy
29,30-dihydroxy
10-phosphate
28-phosphate
40-phosphate
16-O-desmethyl
27-O-desmethyl
39-O-desmethyl
16,27-bis(O-desmethyl)
16,39-bis(O-desmethyl)
27,39-bis(O-desmethyl)
16,27,39-tris(O-desmethyl)
16-desmethoxy
27-desmethoxy
39-O-desmethyl- 14-hydroxy
17,18-epoxide
19,20-epoxide
21,22-epoxide
29,30-epoxide
17,18-29,30-bis-epoxide
17,18-19,20-21,22-tris-epoxide
19,20-21,22-29,30-tris-epoxide
16-O-desmethyl-17,18-19,20-bis-epoxide
16-O-desmethyl-17,18-29,30-bis-epoxide
16-O-desmethyl-17,18-19,20-21,22-tris-epoxide
16-O-desmethyl-19,20-21,22-29,30-tris-epoxide
27-O-desmethyl-17,18-19,20-21,22-tris-epoxide
39-O-desmethyl-17,18-19,20-21,22-tris-epoxide
16,27-bis(O-desmethyl)-17,18-19,20-21,22-tris-epoxide
16-O-desmethyl-24-hydroxy-17,18-19,20-bis-epoxide
16-O-desmethyl-24-hydroxy-17,18-29,30-bis-epoxide
12-hydroxy and opened hemiketal ring

As used herein, an anti-angiogenic agent refers to an agent which reduces or inhibits angiogenesis directly or indirectly. Anti-angiogenic agents include, but are not limited to, anti-VEGF agents, anti-ANG agents, anti-FGF agents, anti-TGF agents, anti-PDGF agents, anti-IGF agents, anti-IL-8 agents, anti-MMP agents, anti-CSF1 agents, anti-PGE2 agents, anti-S1P agents, anti-heparinase agents, anti-NO agents, anti-peroxynitrite agents, anti-serotonin agents, anti-histamine agents, TSP, ANG2, angiostatin, endostatin, vasostatin, calreticulin, CXCL4, TIMP, CDAI, Meth-1, Meth-2, IFN-α, IFN-β, IFN-γ, CXCL10, IL-4, IL-12, IL-18, prolactin, VEGI, SPARC, osteopontin, maspin, canstatin, proliferin-related protein, and combinations thereof.

As used herein, an anti-VEGF agent refers to an agent which selectively or nonselectively inhibit the VEGF pathway including agents which target VEGF-A, VEGF-B, VEGF-C, VEGF-D, PIGF, VEGFR1, VEGFR2, VEGFR3. Anti-VEGF agents include, but are not limited to, ranibizumab, aflibercept, pegaptanib sodium, bevacizumab, brolucizumab, lapatinib, sorafenib, axitinib, pazopanib, sunitinib, cabozantinib, lenvatinib, ponatinib, ramucirumab, regorafenib, vandetanib, cediranib, thiazolidinediones or RGX-314. Preferred anti-VEGF agents include brolucizumab, ranibizumab, aflibercept, and pegaptanib.

As used herein, an anti-platelet agent refers to any substance which inhibits the aggregation of platelets, including platelet aggregation inhibitors and thrombocyte aggregation inhibitors, and/or platelet adhesion inhibitors. Anti-platelet agents include, but are not limited to, acetylsalicylic acid (or aspirin), P2Y12 antagonists such as ticlopidine, clopidogrel, prasugrel, ticagrelor, cangrelor, and elinogrel, PAR-1 antagonists such as vorapaxar, PAR-4 antagonists, EP3 antagonists such as DG041, GPVI antagonists such as soluble dimeric glycoprotein VI-Fc fusion protein, GPIb antagonists, and anti-VWF agents such as caplacizumab, and other antiplatelet drug such as Abciximab, Eptifibatide, Orbofiban, Roxifiban, Sibrafiban, Tirofiban, Beraprost, Iloprost, Prostacyclin, Treprostinil, Acetylsalicylic acid/Aspirin, Aloxiprin, Carbasalate calcium, Indobufen, Triflusal, Dipyridamole/ aspirin, Picotamide, Terbogrel, Terutroban, Cilostazol, Dipyridamole, Triflusal, Cloricromen, Ditazole, Vorapaxar, Ticlopidine, or others; and/or thrombolytic drugs/fibrinolytics drug such as Plasminogen activators r-tPA, Alteplase, Reteplase, Tenecteplase, Desmoteplase, Saruplase, Urokinase, Anistreplase, Monteplase, Streptokinase, Ancrod, Brinase, Fibrinolysin, or others; and/or Citrate, EDTA, Oxalate; and/or an inhibitor for intrinsic pathway of coagulation and thrombosis such as FXIa inhibitor, protein Z-dependent protease inhibitor, Vitamin K antagonist such as Acenocoumarol, Coumatetralyl, Dicoumarol, Ethyl biscoumacetate, Phenprocoumon, Warfarin, Clorindione, Diphenadione, Phenindione, Tioclomarol, or others; and/or other anti-coagulant drug such as Antithrombin III, Defibrotide, Protein C (Drotrecogin alfa), Ramatroban, REG1, or others.

As used here, other therapeutic agents that can be incorporated into the formulations in the invention include but not limit to ranibizumab, aflibercept, pegaptanib sodium, bevacizumab, brolucizumab, lapatinib, sorafenib, axitinib, pazopanib, sunitinib, cabozantinib, lenvatinib, ponatinib, ramucirumab, regorafenib, vandetanib, verteporfin, cediranib, thiazolidinediones, RGX-314, bimatoprost, latanoprost, tafluprost, travoprost, bethamethasone, dexamethasone, methylprednisolone, cortisone, hydrocortisone, triamcinolone, prednisolone, chloramphenicol, sulfacetamide sodium, moxifloxacin, gatifloxacin, erythromycin, polymyxin/trimethoprim, bacitracin, ocumycin, polytracin, nepafenac, bromfenac, salicylate, indomethacin, ibuprofen, diclofenac, flurbiprofen, naproxen, piroxicam,nabumetone, sulfonamides, sulfacetamide, sulfamethizole, sulfisoxazole; nitrofurazone, sodium propionate, antazoline, methapyriline, chlorpheniramine, pyrilamine, prophenpyridamine, pilocarpine, eserine salicylate, carbachol, di-isopropyl fluorophosphate, phospholine iodine, demecarium bromide, atropine sulfate, cyclopentolate, homatropine, scopolamine, tropicamide, eucatropine, hydroxy amphetamine, and the like or a salt, isomer, solvate, analog, derivative, metabolite, or prodrugs thereof.

Described herein are devices, compositions, and methods for inhibiting clot formation, fibrin, fibrosis, and/or inflammation and/or glaucoma, and/or AMD of ophthalmic disease or condition in a patient. A therapeutic composition may be provided as described herein. A therapeutically effective dose of the therapeutic composition may be delivered to the site of the disease or condition in the patient’s eye(s). The therapeutically effective dose of the therapeutic composition may be effective to suppress or prevent initiation, progression, or relapses of disease, including the progression of established disease. In some examples, the ophthalmic disease or condition comprises one or more of angiogenesis, leaking blood vessel, protein deposition, fibrosis, glaucoma, extracellular matrix (ECM) deposition, thrombin generation, clot formation, or fibrin formation. For example, the ophthalmic disease or condition may be wet age-related macular degeneration (AMD), dry AMD, diabetic retinopathy, glaucoma, uveitis, cataracts, conjunctivitis, or dry eye disease. In some examples, the patient may be diagnosed as having the ophthalmic disease prior to delivering the therapeutically effective dose of the therapeutic composition.

Tissue plasminogen activator (tPA) is one of the essential components of the dissolution of blood clots. Its primary function includes catalyzing the conversion of plasminogen to plasmin, the primary enzyme involved in dissolving blood clots. Since fibrin clots can occur in several sites of the body, including the eye, the tPA therapy could be effective for the rapid dissolution of fibrin clots in the anterior chamber of the human eye, as well as for lysis of fibrin clots after vitrectomy and failed blebs after glaucoma filtering surgery.

Tissue plasminogen activator (tPA) can dissolve any fibrin material that forms in the anterior chamber or vitreous humor. Ocular tissues that contain tPA include the conjunctiva, cornea, trabecular meshwork, lens, vitreous, and retina. In normal human eyes, the aqueous humour contains a significant amount of tPA; however, for disease eyes related to tPA deficiency, medication tPA could be considered.

Since fibrin clots can occur in several sites of the body, including the eye, the tPA therapy could be effective for the rapid dissolution of fibrin clots in the anterior chamber of the human eye, as well as for lysis of fibrin clots after vitrectomy and failed blebs after glaucoma filtering surgery. The thrombolytic drugs comprise tissue plasminogen activator( t-PA), urokinase-type plasminogen activator (u-PA), Streptokinase or plasmin.

The serine protease agent comprises one of t-PA, U-PA, or plasmin, or other agents that has similar mechanism of action. The tPA therapeutic agent can be, for example, tPA, a tPA derivative, a small molecule direct or indirect tPA agonist, or a gene therapy vector. The tPA eye drops administered topically to patients with postoperative hyphema could give rapid clearance of the clot. Intracameral tPA injection is a safe and effective way to help lyse fibrin and dissolve blood clots to reopen occluded glaucoma drainage devices. tPA is a nonsurgical eye injection that’s often used to treat patients suffering from wet age-related macular degeneration (AMD). It’s also used to treat a common side effect of AMD, submacular hemorrhages. The tPA eye drops or injections can benefit eye disease such as vitreous opacity, wet age-related macular degeneration (AMD), glaucoma and anterior chamber fibrinoid syndrome after cataract extraction etc. tPA use could have prevented the need for additional glaucoma surgery. One or more injections of tPA may have been used to clear or prevent tube blockage by blood and/or fibrin to result in a successful ocular outcome.

In a preferred example the agent is present in the therapeutic composition at a concentration within a range of 1 mg/ml to about 250 mg/ml. In some examples, the serine protease agent comprises t-PA and the therapeutically effective dose comprises a range of about 50 µg to about 5 mg, about 25 µg to about 5 mg, or about 0.3 µg to about 3 mg.

Ocular inflammation is a sight threating disease and its dysregulation can catalyze and or propagate ocular neurodegenerative maladies such as age-related macular degeneration (AMD). The complement system, an intrinsic component of the innate immunity, has an integral role in maintaining immune-surveillance and homeostasis in the ocular microenvironment; however, overstimulation can drive ocular inflammatory diseases. The incidence of advanced AMD, both geographic atrophy and neovascular AMD, increases exponentially with age and while there are therapies for wet AMD, Geographic atrophy (GA) currently has no approved treatment options. The aim of most current clinical trials is to reduce the progression of GA lesion enlargement.

Complement is the main driver of disease for AMD, Uveitis, Glaucoma. Local inhibition of complement activation has been considered a promising approach for treating these diseases. Complement pathway has three pathways which involves various factors, B, D, H & I, which interact with each other, and with C3b, to form a C3 convertase, C3bBb, that can activate more C3, hence the pathway is sometimes called ‘the amplification loop’. C3 is the converging point for all 3 complement pathways. Complement inhibitors target the modulation of complement proteins C3, C5, factor B, factor D, and properdin.

In some examples, the complement-mediated therapeutics include but not limit to C3 inhibitor such as APL-2,Compstatin and AMY106, Anti-factor D such as Lampalizumab, Anti-Properdin such as CLG561, Anti-C5 such as Eculizumab, Ravulizumab,ARC1905, and LFG316, CD59 such as AAVCAGsCD59, analogues and the like.

In some examples, the complement inhibitor is Compstatin or its analogue. Compstatin is a 13 amino acid cyclic peptide that inhibits complement activation by binding C3 and interfering with convertase formation and C3 cleavage (and the subsequent C3b opsonization, amplification, and generation of effectors). Compstatin could be a potent inhibitor, blocking all three complement systems.

In some examples, the complement inhibitor is Anti-C5 such as Eculizumab and ravulizumab. Eculizumab or Ravulizumab specifically binds to the terminal complement component 5, or C5, which acts at a late stage in the complement cascade. When activated, C5 is involved in activating host cells, thereby attracting pro-inflammatory immune cells, while also destroying cells by triggering pore formation. By inhibiting the complement cascade at this point, the normal, disease-preventing functions of proximal complement system are largely preserved, while the properties of C5 that promote inflammation and cell destruction are impeded.

In some examples, the eye injections comprise complement inhibitor Compstatin or Eculizumab or Ravulizumab or its analogue, which can benefit eye disease such as AMD, Uveitis, Glaucoma, etc.

In some examples, the eye injections comprise complement inhibitor Compstatin or Eculizumab or Ravulizumab or its analogue with combination of direct factor Xa inhibitor Apixaban and direct factor IIa inhibitor Argatroban. This combination therapy will has a big impact on ocular inflammation and may be efficient to eye disease such as AMD, Uveitis and Glaucoma. The rate of progression of atrophy in the eyes of patients with dry AMD might be slowed down or stopped with combination of Eculizumab or Ravulizumab and direct factor Xa inhibitor Apixaban and direct factor IIa inhibitor Argatroban, which otherwise has no treatment available.

Inflammation, fibrosis, and/or clot formation of the ocular tissue caused by the ophthalmic disease or condition may lead to fluid build-up, thrombin generation, fibrosis, and/or fibrin formation in the eye, which can damage tissue in the eye. Ocular tissues include, but are not limited to, an eyelid, a meibomian gland, a trabecular meshwork, Schlemm’s canal, a cornea, a sclera, a vitreous, a choroid, a uvea, and/or a retina.

In some examples, the therapeutic composition is locally delivered to the eye. In some examples, the therapeutic composition is delivered to ocular tissue including an eyelid, a meibomian gland, a trabecular meshwork, Schlemm’s canal, a cornea, a sclera, a vitreous, a choroid, a uvea, and/or a retina. The therapeutic composition may be delivered to the eye by any method or device for ocular delivery. For example, the therapeutic composition may be delivered with an injection, an implant, an eye drop, an emulsion, a suspension, an ointment, a nanocarrier, a nanomicelle, a nanoparticle, a nanosuspension, a liposome, an in-situ gel, a contact lens, or a microneedle.

When an implant is utilized, the implant can be implanted intraocularly or intravitreally by an intervention procedure. Such implants can be non-biodegradable, biodegradable, removable or permanent. In other examples, implants can be a shunt or stent placed in the duct, such as the tear duct. In still other examples, implants can be placed adjacent to the ocular body, or intraocularly, adjacent to the vitreal body or intravitreally. One of ordinary skill in the art will appreciate that other locations are useful in the present disclosure.

In some examples, the therapeutic composition may be delivered to the eye(s) by one or more methods or devices. For example, a first portion (e.g., one or more bioactive agents) of the therapeutic composition may be delivered to the eye with a first device (e.g., an implant) and a second portion (e.g., one or more additional pharmaceutical agents) of the therapeutic composition may be delivered to the eye with a second device (e.g., a second implant, a contact lens, etc.) before or after delivery of the first portion of the therapeutic composition. Alternatively, all components (e.g., all bioactive agents) of the therapeutic composition may be delivered with the same device (e.g., an implant). It will be understood by one of ordinary skill in the art that the therapeutic composition may comprise one or many components which may be delivered simultaneously or sequentially in any order or combination as desired with any combination of methods or devices desired.

In some examples, local delivery of the therapeutic composition may be preferable to systemic delivery of the therapeutic composition in at least some instances. For example, local delivery of the therapeutic composition may reduce off-target effects by reducing systemic concentrations and/or increase the tissue concentration of the therapeutic composition at the target site compared to what can be achieved with systemic delivery safety maximums.

In some examples, the therapeutic composition may comprise one or more of a pharmaceutically acceptable carrier, a diluent, an adjuvant, an excipient, a vehicle, a preserving agent, a filler, a polymer, a disintegrating agent, a glidant, a wetting agent, an emulsifying agent, a suspending agent, a sweetening agent, a flavoring agent, a perfuming agent, a lubricating agent, an acidifying agent, and a dispensing agent.

In some examples, the therapeutic composition may comprise or be coadministered with one or more additional bioactive agents which address or treat the underlying disease or infection in order to improve therapeutic outcome.

While many of the examples described herein depict the therapeutic composition being in the form of an eye drop for local delivery, it will be understood by one of ordinary skill in the art that local delivery may be achieved through any other means desired. For examples, the therapeutic composition may be coated, dipped, printed, deposited, painted, brushed, loaded, or otherwise disposed on one or more surfaces of a device for local delivery. In some examples, the therapeutic composition may be locally delivered via direct injection using a device (e.g., a needle, a microneedle, etc.) comprising or coupled to a drug reservoir.

It is an objective of this application to show that ophthalmic delivery of the direct factor Xa inhibitor and/or additional therapeutically active substance (e.g., an anti-coagulant such as a direct factor IIa inhibitor), and optionally in combination with one or more additional pharmaceutical agent, inhibits one or more of inflammation, thrombin formation, fibrin formation, clot formation, smooth muscle proliferation, fibrosis, and/or adverse clinical events.

It is an objective of this application to show that opthalmic delivery of a pharmaceutical agent to inhibits one or more of inflammation, fibrosis, and/or adverse clinical events.

Described herein are devices and methods for locally delivering a therapeutic composition to a patient. The therapeutic composition includes one or more anti-coagulant agents which inhibit thrombin, fibrin, and/or thrombus formation or promote thrombin, fibrin, and/or thrombus dissolution. In preferred examples, the therapeutic composition includes a direct Xa inhibitor. In another preferred example, the therapeutic composition includes a direct Xa inhibitor and/or a direct IIa inhibitor. In another preferred example, one or more additional pharmaceutical agents may be added to the therapeutic composition of the direct Xa inhibitor and/or the direct IIa inhibitor. The additional pharmaceutical agents may, for example, include one or more anti-fibrotic agents, metformin, anti-angiogenic agents, anti-VEGF, anti-proliferative agents, or combinations thereof.

In some examples, the pharmaceutical agents may, for example, include one or more anti-fibrotic agents, metformin, metformin hydroxy chloride (HCl), anti-angiogenic agents, anti-proliferative agents, or combinations thereof.

In some examples, the pharmaceutical agent may, for example, include one or more anti-viral/anti-diabetic agents such as metformin or its salt metformin HCl. Metformin was originally introduced as an anti-influenza drug and was found to have glucose-lowering side effects, making it effective as both an anti-viral and an anti-diabetic agent. Metformin activates AMP-activated protein kinase (AMPK) which, among other things, phosphorylates and upregulates angiotensin-converting enzyme 2 (ACE2) and inhibits the mTOR signaling cascade. The anti-viral activity of metformin may, in at least some instances, be related to these activities. Metformin can also be used for treatment of ophthalmic disorders. For example, metformin may be used for treatment of diabetic retinopathy and/or macular edema. In at least some instances, metformin may play a protective role in microvascular ocular complications, including retinopathy, glaucoma, and/or age-related macular degeneration (AMD) in patients with type-2 diabetes. For example, metformin inhibits development of diabetic retinopathy by inducing microRNA-497a-5p, a VEGF-A-inhibiting microRNA, resulting in reduced retina neovascularization.

In some examples, the therapeutic composition comprises a direct factor Xa inhibitor, a additional therapeutically active substance (e.g., an anti-coagulant), and/or one or more additional pharmaceutical agent(s) (e.g., an anti-fibrotic agent, an anti-viral agent, an anti-diabetic agent, an anti-angiogenic agent, and/or an anti-proliferative agent).

In some examples, the therapeutic composition comprises a direct factor Xa inhibitor, a direct factor IIa inhibitor, and/or an anti-fibrotic agent. In some examples, the therapeutic composition may comprise a direct factor Xa inhibitor, a direct factor IIa inhibitor, and an anti-fibrotic agent. In some examples, the therapeutic composition may comprise a direct factor Xa inhibitor and a direct factor IIa inhibitor but no anti-fibrotic agent. In some examples, the therapeutic composition may comprise a direct factor Xa inhibitor and an anti-fibrotic agent but no direct factor IIa inhibitor. In some examples, the therapeutic composition may comprise a direct factor Xa inhibitor but no direct factor IIa inhibitor or anti-fibrotic agent.

In some examples, the direct factor Xa inhibitor, direct factor IIa inhibitor, and/or anti-fibrotic agent may be delivered simultaneously or sequentially in any order or combination as desired with any combination of methods or devices desired. For example, the same eye drop may be used to deliver the direct factor Xa inhibitor, the direct factor IIa inhibitor, and the anti-fibrotic agent. Alternatively, a first eye dropper may be used to deliver the direct factor Xa inhibitor and a second eye dropper may be used to deliver the direct factor IIa inhibitor and the anti-fibrotic agent. Alternatively, a first eye dropper may be used to deliver the direct factor Xa inhibitor and the direct factor IIa inhibitor and a second eye dropper may be used to deliver the anti-fibrotic agent. Alternatively, each agent may be delivered by its own dedicated eye dropper.

In some examples, the therapeutic composition comprises a direct factor Xa inhibitor, a direct factor IIa inhibitor, and/or an anti-viral/anti-diabetic agent. In some examples, the therapeutic composition may comprise a direct factor Xa inhibitor, a direct factor IIa inhibitor, and anti-viral/anti-diabetic agent. In some examples, the therapeutic composition may comprise a direct factor Xa inhibitor and a direct factor IIa inhibitor but no anti-viral/anti-diabetic agent. In some examples, the therapeutic composition may comprise a direct factor Xa inhibitor and an anti-viral/anti-diabetic agent but no direct factor IIa inhibitor. In some examples, the therapeutic composition may comprise a direct factor Xa inhibitor but no direct factor IIa inhibitor or anti-viral/anti-diabetic agent.

In some examples, the direct factor Xa inhibitor, direct factor IIa inhibitor, and/or anti-viral/anti-diabetic agent may be delivered simultaneously or sequentially in any order or combination as desired with any combination of methods or devices desired. For example, the same eye dropper may be used to deliver the direct factor Xa inhibitor, the direct factor IIa inhibitor, and the anti-viral/anti-diabetic agent. Alternatively, a first eye dropper may be used to deliver the direct factor Xa inhibitor and a second eye dropper may be used to deliver the direct factor IIa inhibitor and the anti-viral/anti-diabetic agent. Alternatively, a first eye dropper may be used to deliver the direct factor Xa inhibitor and the direct factor IIa inhibitor and a second eye dropper may be used to deliver the anti-viral/anti-diabetic agent. Alternatively, each agent may be delivered by its own dedicated eye dropper.

In some examples, the therapeutic composition comprises a direct factor Xa inhibitor, a direct factor IIa inhibitor, and/or an anti-proliferative agent. In some examples, the therapeutic composition may comprise a direct factor Xa inhibitor, a direct factor IIa inhibitor, and an anti-proliferative agent. In some examples, the therapeutic composition may comprise a direct factor Xa inhibitor and a direct factor IIa inhibitor but no anti-proliferative agent. In some examples, the therapeutic composition may comprise a direct factor Xa inhibitor and an anti-proliferative agent but no direct factor IIa inhibitor. In some examples, the therapeutic composition may comprise a direct factor Xa inhibitor but no direct factor IIa inhibitor or anti-proliferative agent.

In some examples, the direct factor Xa inhibitor, direct factor IIa inhibitor, and/or anti-proliferative agent may be delivered simultaneously or sequentially in any order or combination as desired with any combination of methods or devices desired. For example, the same eye dropper may be used to deliver the direct factor Xa inhibitor, the direct factor IIa inhibitor, and the anti-proliferative agent. Alternatively, a first eye dropper may be used to deliver the direct factor Xa inhibitor and a second eye dropper may be used to deliver the direct factor IIa inhibitor and the anti-proliferative agent. Alternatively, a first eye dropper may be used to deliver the direct factor Xa inhibitor and the direct factor IIa inhibitor and a second eye dropper may be used to deliver the anti-proliferative agent. Alternatively, each agent may be delivered by its own dedicated eye dropper.

In some examples, the therapeutic composition comprises a direct factor Xa inhibitor, a direct factor IIa inhibitor, and/or an anti-angiogenic agent. In some examples, the therapeutic composition may comprise a direct factor Xa inhibitor, a direct factor IIa inhibitor, and an anti-angiogenic agent. In some examples, the therapeutic composition may comprise a direct factor Xa inhibitor and a direct factor IIa inhibitor but no anti-angiogenic agent. In some examples, the therapeutic composition may comprise a direct factor Xa inhibitor and an anti-angiogenic agent but no direct factor IIa inhibitor. In some examples, the therapeutic composition may comprise a direct factor Xa inhibitor but no direct factor IIa inhibitor or anti-angiogenic agent.

In some examples, the direct factor Xa inhibitor, direct factor IIa inhibitor, and/or anti-angiogenic agent may be delivered simultaneously or sequentially in any order or combination as desired with any combination of methods or devices desired. For example, the same eye dropper may be used to deliver the direct factor Xa inhibitor, the direct factor IIa inhibitor, and the anti-angiogenic agent. Alternatively, a first eye dropper may be used to deliver the direct factor Xa inhibitor and a second eye dropper may be used to deliver the direct factor IIa inhibitor and the anti-angiogenic agent. Alternatively, a first eye dropper may be used to deliver the direct factor Xa inhibitor and the direct factor IIa inhibitor and a second eye dropper may be used to deliver the anti-angiogenic agent. Alternatively, each agent may be delivered by its own dedicated eye dropper.

When the therapeutic composition comprises a direct factor Xa inhibitor and a direct factor IIa inhibitor, the weight compositional ratio of the direct factor Xa inhibitor to the direct factor IIa inhibitor in the therapeutic composition may be within a range of about 3:1 to about 1:3. For example, the weight compositional ratio of the direct factor Xa inhibitor to the direct factor IIa inhibitor in the therapeutic composition may be about 1:1.

In some examples, the direct factor Xa inhibitor may have an IC50 inhibition potency for factor Xa ranging from about 0.001 nM to about 50 nM.

In some examples, the direct factor IIa inhibitor may have an IC50 inhibition potency for factor IIa ranging from about 0.001 nM to about 100 nM.

In some examples, the direct factor Xa inhibitor may have a half maximal inhibitory concentration (IC₅₀) within a range of about 0.01 nM to about 1000 nM, about 0.1 nM to about 1000 nM, about 1 nM to about 1000 nM, 10 nM to about 1000 nM, or about 100 nM to about 1000 nM.

In some examples, the direct factor IIa inhibitor may have an inhibition potency for factor IIa ranging from about 0.001 nM to about 100 nM.

In some examples, the direct factor IIa inhibitor may have a half maximal inhibitory concentration (IC₅₀) within a range of about 0.01 nM to about 1000 nM, about 0.1 nM to about 1000 nM, about 1 nM to about 1000 nM, 10 nM to about 1000 nM, or about 100 nM to about 1000 nM.

In some examples, the direct factor IIa inhibitor may comprise argatroban, or a salt, isomer, solvate, analog (including deuterated analog), derivative, metabolite, or prodrugs thereof.

In some examples, the direct factor Xa inhibitor may comprise apixaban, or a salt, isomer, solvate, analog (including deuterated analog), derivative, metabolite, or prodrugs thereof.

In some examples, the direct factor Xa inhibitor may comprise rivaroxaban, or a salt, isomer, solvate, analog (including deuterated analog), derivative, metabolite, or prodrugs thereof.

In some examples, the anti-fibrotic agent may comprise nintedanib, or a salt, isomer, solvate, analog (including deuterated analog), derivative, metabolite, or prodrugs thereof.

In some examples, the anti-fibrotic agent may comprise pirfenidone, or a salt, isomer, solvate, analog (including deuterated analog), derivative, metabolite, or prodrugs thereof.

In some examples, the anti-viral/anti-diabetic agent may comprise metformin, or a salt, isomer, solvate, analog (including deuterated analog), derivative, metabolite, or prodrugs thereof.

In some examples, the anti-proliferative agent may comprise sirolimus, or a salt, isomer, solvate, analog (including deuterated analog), derivative, metabolite, or prodrugs thereof.

In some examples, the anti-proliferative agent may comprise novolimus, or a salt, isomer, solvate, analog (including deuterated analog), derivative, metabolite, or prodrugs thereof.

In some examples, the anti-proliferative agent may comprise myolimus, or a salt, isomer, solvate, analog (including deuterated analog), derivative, metabolite, or prodrugs thereof.

In some examples, the anti-angiogenic agent may comprise ranibizumab, or a salt, isomer, solvate, analog (including deuterated analog), derivative, metabolite, or prodrugs thereof.

In some examples, the anti-angiogenic agent may comprise brolucizumab, or a salt, isomer, solvate, analog (including deuterated analog), derivative, metabolite, or prodrugs thereof.

In some examples, the anti-angiogenic agent may comprise aflibercept, or a salt, isomer, solvate, analog (including deuterated analog), derivative, metabolite, or prodrugs thereof.

In some examples, the anti-angiogenic agent may comprise pegaptanib, or a salt, isomer, solvate, analog (including deuterated analog), derivative, metabolite, or prodrugs thereof.

In some examples, the direct factor Xa inhibitor may comprise apixaban and the direct factor IIa inhibitor may comprise argatroban.

In some examples, the direct factor Xa inhibitor may comprise rivaroxaban and the direct factor IIa inhibitor may comprise argatroban.

In some examples, the direct factor Xa inhibitor may comprise apixaban, the direct factor IIa inhibitor may comprise argatroban, and the anti-fibrotic agent may comprise nintedanib.

In some examples, the direct factor Xa inhibitor may comprise apixaban, the direct factor IIa inhibitor may comprise argatroban, and the anti-fibrotic agent may comprise pirfenidone.

In some examples, the direct factor Xa inhibitor may comprise rivaroxaban, the direct factor IIa inhibitor may comprise argatroban, and the anti-fibrotic agent may comprise nintedanib.

In some examples, the direct factor Xa inhibitor may comprise rivaroxaban, the direct factor IIa inhibitor may comprise argatroban, and the anti-fibrotic agent may comprise pirfenidone.

In some examples, the direct factor Xa inhibitor may comprise apixaban, the direct factor IIa inhibitor may comprise argatroban, and the anti-diabetic agent may comprise metformin or its salt.

In some examples, the direct factor Xa inhibitor may comprise rivaroxaban, the direct factor IIa inhibitor may comprise argatroban, and the anti-diabetic agent may comprise metformin or its salt.

In some examples, the direct factor Xa inhibitor may comprise apixaban, the direct factor IIa inhibitor may comprise argatroban, and the anti-proliferative agent may comprise sirolimus or its isomer, solvate, analog, derivative, metabolite, or prodrugs.

In some examples, the direct factor Xa inhibitor may comprise apixaban, the direct factor IIa inhibitor may comprise argatroban, and the anti-proliferative agent may comprise novolimus or its isomer, solvate, analog, derivative, metabolite, or prodrugs.

In some examples, the direct factor Xa inhibitor may comprise apixaban, the direct factor IIa inhibitor may comprise argatroban, and the anti-proliferative agent may comprise myolimus or its isomer, solvate, analog, derivative, metabolite, or prodrugs.

In some examples, the direct factor Xa inhibitor may comprise rivaroxaban, the direct factor IIa inhibitor may comprise argatroban, and the anti-proliferative agent may comprise sirolimus or its isomer, solvate, analog, derivative, metabolite, or prodrugs.

In some examples, the direct factor Xa inhibitor may comprise rivaroxaban, the direct factor IIa inhibitor may comprise argatroban, and the anti-proliferative agent may comprise novolimus or its isomer, solvate, analog, derivative, metabolite, or prodrugs.

In some examples, the direct factor Xa inhibitor may comprise rivaroxaban, the direct factor IIa inhibitor may comprise argatroban, and the anti-proliferative agent may comprise myolimus or its isomer, solvate, analog, derivative, metabolite, or prodrugs.

In some examples, the direct factor Xa inhibitor may comprise rivaroxaban, the direct factor IIa inhibitor may comprise argatroban, and the anti-angiogenic agent may comprise ranibizumab.

In some examples, the direct factor Xa inhibitor may comprise rivaroxaban, the direct factor IIa inhibitor may comprise argatroban, and the anti-angiogenic agent may comprise brolucizumab.

In some examples, the direct factor Xa inhibitor may comprise rivaroxaban, the direct factor IIa inhibitor may comprise argatroban, and the anti-angiogenic agent may comprise aflibercept.

In some examples, the direct factor Xa inhibitor may comprise rivaroxaban, the direct factor IIa inhibitor may comprise argatroban, and the anti-angiogenic agent may comprise pegaptanib.

In some examples, the direct factor Xa inhibitor may comprise apixaban, the direct factor IIa inhibitor may comprise argatroban, and the anti-angiogenic agent may comprise ranibizumab.

In some examples, the direct factor Xa inhibitor may comprise apixaban, the direct factor IIa inhibitor may comprise argatroban, and the anti-angiogenic agent may comprise brolucizumab.

In some examples, the direct factor Xa inhibitor may comprise apixaban, the direct factor IIa inhibitor may comprise argatroban, and the anti-angiogenic agent may comprise aflibercept.

In some examples, the direct factor Xa inhibitor may comprise apixaban, the direct factor IIa inhibitor may comprise argatroban, and the anti-angiogenic agent may comprise pegaptanib.

In some examples, the anti-proliferative agent comprises sirolimus and is present in the therapeutic composition at a concentration within a range of about 1 mg/ml to about 250 mg/ml, 10 mg/ml to about 250 mg/ml, or 100 mg/ml to about 250 mg/ml.

In some examples, the anti-proliferative agent comprises novolimus and is present in the therapeutic composition at a concentration within a range of about 1 mg/ml to about 250 mg/ml, 10 mg/ml to about 250 mg/ml, or 100 mg/ml to about 250 mg/ml.

In some examples, the anti-proliferative agent comprises myolimus and is present in the therapeutic composition at a concentration within a range of about 1 mg/ml to about 250 mg/ml, 10 mg/ml to about 250 mg/ml, or 100 mg/ml to about 250 mg/ml.

In some examples, the anti-angiogenic agent comprises brolucizumab and is present in the therapeutic composition at a concentration within a range of about 50 mg/ml to about 500 mg/ml.

In some examples, the anti-angiogenic agent comprises ranibizumab and is present in the therapeutic composition at a concentration within a range of about 6 mg/ml to about 40 mg/ml.

In some examples, the anti-angiogenic agent comprises aflibercept and is present in the therapeutic composition at a concentration within a range of about 10 mg/ml to about 100 mg/ml.

In some examples, the anti-angiogenic agent comprises pegaptanib and is present in the therapeutic composition at a concentration within a range of about 1 mg/ml to about 10 mg/ml.

In some examples, a therapeutically effective dose of the direct factor Xa inhibitor is within a range of about 50 micrograms to about 10 mg. In some examples, the therapeutically effective dose of the direct factor Xa inhibitor is within a range of about 0.1 mg to about 10 mg. In some examples, the therapeutically effective dose of the direct factor Xa inhibitor is within a range of about 1 mg to about 5 mg.

In some examples, a therapeutically effective dose of the direct factor IIa inhibitor is within a range of about 50 micrograms to about 10 mg. In some examples, the therapeutically effective dose of the direct factor IIa inhibitor is within a range of about 0.1 mg to about 10 mg. In some examples, the therapeutically effective dose of the direct factor IIa inhibitor is within a range of about 1 mg to about 5 mg.

In some examples, the anti-fibrotic agent comprises nintedanib and a therapeutically effective dose of nintedanib is within a range of about 0.1 mg to about 5 mg.

In some examples, the anti-proliferative agent comprises sirolimus and a therapeutically effective dose of sirolimus is within a range of about 50 µg to about 5 mg, about 0.1 mg to about 5 mg, or about 0.5 mg to about 3 mg.

In some examples, the anti-proliferative agent comprises novolimus and a therapeutically effective dose of novolimus is within a range of about 50 µg to about 5 mg, about 0.1 mg to about 5 mg, or about 0.5 mg to about 3 mg.

In some examples, the anti-proliferative agent comprises myolimus and a therapeutically effective dose of myolimus is within a range of about 50 µg to about 5 mg, about 0.1 mg to about 5 mg, or about 0.5 mg to about 3 mg.

In some examples, the anti-angiogenic agent comprises brolucizumab and a therapeutically effective dose of brolucizumab is within a range of about 0.3 mg to about 30 mg.

In some examples, the anti-angiogenic agent comprises ranibizumab and a therapeutically effective dose of ranibizumab is within a range of about 0.3 mg to about 2 mg.

In some examples, the anti-angiogenic agent comprises aflibercept a therapeutically effective dose of aflibercept is within a range of about 0.5 mg to about 5 mg.

In some examples, the anti-angiogenic agent comprises pegaptanib and a therapeutically effective dose of pegaptanib is within a range of about 0.1 mg to about 1 mg.

In some examples, the direct factor Xa inhibitor is present in the therapeutic composition at a concentration effective to achieve a tissue concentration of the direct factor Xa inhibitor of about 0.1 ng/g tissue to about 100 mg/g tissue when a therapeutically effective dose of the therapeutic composition is delivered to the tissue. In some examples, the therapeutically effective dose may be sufficient to generate a tissue concentration of the direct factor Xa inhibitor of about 0.2 ng/g tissue to about 100 mg/g tissue. In some examples, the therapeutically effective dose may be sufficient to generate a tissue concentration of the direct factor Xa inhibitor of 0.5 ng/g tissue to about 100 mg/g tissue. In some examples, the therapeutically effective dose may be sufficient to generate a tissue concentration of the direct factor Xa inhibitor of about 1 ng/g tissue to about 100 mg/g tissue. In some examples, the therapeutically effective dose may be sufficient to generate a tissue concentration of the direct factor Xa inhibitor of about 10 ng/g tissue to about 100 mg/g tissue. In some examples, the therapeutically effective dose may be sufficient to generate a tissue concentration of the direct factor Xa inhibitor of about 100 ng/g tissue to about 100 mg/g tissue.

In some examples, the therapeutically effective dose is sufficient to maintain a tissue concentration of the direct factor Xa inhibitor of about 0.1 ng/g tissue to about 100 mg/g tissue for about 1 day to about 1 year, 30 days to about 1 year, 3 months to about 1 year, or 6 months to about 1 year.

In some examples, the direct factor IIa inhibitor is present in the therapeutic composition at a concentration effective to achieve a tissue concentration of the direct factor IIa inhibitor of about 0.1 ng/g tissue to about 100 mg/g tissue when a therapeutically effective dose of the therapeutic composition is delivered to the tissue. In some examples, the therapeutically effective dose may be sufficient to generate a tissue concentration of the direct factor IIa inhibitor of about 0.2 ng/g tissue to about 100 mg/g tissue. In some examples, the therapeutically effective dose may be sufficient to generate a tissue concentration of the direct factor IIa inhibitor of 0.5 ng/g tissue to about 100 mg/g tissue. In some examples, the therapeutically effective dose may be sufficient to generate a tissue concentration of the direct factor IIa inhibitor of about 1 ng/g tissue to about 100 mg/g tissue. In some examples, the therapeutically effective dose may be sufficient to generate a tissue concentration of the direct factor IIa inhibitor of about 10 ng/g tissue to about 100 mg/g tissue. In some examples, the therapeutically effective dose may be sufficient to generate a tissue concentration of the direct factor IIa inhibitor of about 100 ng/g tissue to about 100 mg/g tissue.

In some examples, the therapeutically effective dose is sufficient to maintain a tissue concentration of the direct factor IIa inhibitor of about 0.1 ng/g tissue to about 100 mg/g tissue for about 1 day to about 1 year, 30 days to about 1 year, 3 months to about 1 year, or 6 months to about 1 year.

In some examples, the anti-fibrotic agent comprises nintedanib and is present in the therapeutic composition at a concentration effective to achieve a tissue concentration of the anti-fibrotic agent of about 0.1 ng/g tissue to about 100 mg/g tissue when a therapeutically effective dose of the therapeutic composition is delivered to the tissue. In some examples, the therapeutically effective dose may be sufficient to generate a tissue concentration of the anti-fibrotic agent of about 0.2 ng/g tissue to about 100 mg/g tissue. In some examples, the therapeutically effective dose may be sufficient to generate a tissue concentration of the anti-fibrotic agent of 0.5 ng/g tissue to about 100 mg/g tissue. In some examples, the therapeutically effective dose may be sufficient to generate a tissue concentration of the anti-fibrotic agent of about 1 ng/g tissue to about 100 mg/g tissue. In some examples, the therapeutically effective dose may be sufficient to generate a tissue concentration of the anti-fibrotic agent of about 100 ng/g tissue to about 100 mg/g tissue. In some examples, the therapeutically effective dose may be sufficient to generate a tissue concentration of the anti-fibrotic agent of about 100 ng/g tissue to about 100 mg/g tissue.

In some examples, the therapeutically effective dose is sufficient to maintain a tissue concentration of nintedanib of about 0.1 ng/g tissue to about 100 mg/g tissue for about 1 day to about 1 year, 30 days to about 1 year, 3 months to about 1 year, or 6 months to about 1 year.

In some examples, the anti-fibrotic agent comprises pirfenidone and is present in the therapeutic composition at a concentration effective to achieve a tissue concentration of the anti-fibrotic agent of about 0.1 ng/g tissue to about 100 mg/g tissue when a therapeutically effective dose of the therapeutic composition is delivered to the tissue. In some examples, the therapeutically effective dose may be sufficient to generate a tissue concentration of the anti-fibrotic agent of about 0.2 ng/g tissue to about 100 mg/g tissue. In some examples, the therapeutically effective dose may be sufficient to generate a tissue concentration of the anti-fibrotic agent of 0.5 ng/g tissue to about 100 mg/g tissue. In some examples, the therapeutically effective dose may be sufficient to generate a tissue concentration of the anti-fibrotic agent of about 1 ng/g tissue to about 100 mg/g tissue. In some examples, the therapeutically effective dose may be sufficient to generate a tissue concentration of the anti-fibrotic agent of about 10 ng/g tissue to about 100 mg/g tissue. In some examples, the therapeutically effective dose may be sufficient to generate a tissue concentration of the anti-fibrotic agent of about 100 ng/g tissue to about 100 mg/g tissue.

In some examples, the therapeutically effective dose is sufficient to maintain a tissue concentration of pirfenidone of about 0.1 ng/g tissue to about 100 mg/g tissue for about 1 day to about 1 year, 30 days to about 1 year, 3 months to about 1 year, or 6 months to about 1 year.

In some examples, the anti-viral/anti-diabetic agent comprises metformin or its salt and is present in the therapeutic composition at a concentration effective to achieve a tissue concentration of the anti-viral/anti-diabetic agent of about 0.1 ng/g tissue to about 100 mg/g tissue when a therapeutically effective dose of the therapeutic composition is delivered to the tissue. In some examples, the therapeutically effective dose may be sufficient to generate a tissue concentration of the anti-viral/anti-diabetic agent of about 0.2 ng/g tissue to about 100 mg/g tissue. In some examples, the therapeutically effective dose may be sufficient to generate a tissue concentration of the anti-viral/anti-diabetic agent of 0.5 ng/g tissue to about 100 mg/g tissue. In some examples, the therapeutically effective dose may be sufficient to generate a tissue concentration of the anti-viral/anti-diabetic agent of about 1 ng/g tissue to about 100 mg/g tissue. In some examples, the therapeutically effective dose may be sufficient to generate a tissue concentration of the anti-viral/anti-diabetic agent of about 10 ng/g tissue to about 100 mg/g tissue. In some examples, the therapeutically effective dose may be sufficient to generate a tissue concentration of the anti-viral/anti-diabetic agent of about 100 ng/g tissue to about 100 mg/g tissue.

In some examples, the therapeutically effective dose is sufficient to maintain a tissue concentration of metformin or its salt of about 0.1 ng/g tissue to about 100 mg/g tissue for about 1 day to about 1 year, 30 days to about 1 year, 3 months to about 1 year, or 6 months to about 1 year.

In some examples, the anti-proliferative agent comprises sirolimus and is present in the therapeutic composition at a concentration effective to achieve a tissue concentration of the anti-proliferative agent of about 0.1 ng/g tissue to about 100 mg/g tissue when a therapeutically effective dose of the therapeutic composition is delivered to the tissue. In some examples, the therapeutically effective dose may be sufficient to generate a tissue concentration of the anti-proliferative agent of about 0.2 ng/g tissue to about 100 mg/g tissue. In some examples, the therapeutically effective dose may be sufficient to generate a tissue concentration of the anti-proliferative agent of 0.5 ng/g tissue to about 100 mg/g tissue. In some examples, the therapeutically effective dose may be sufficient to generate a tissue concentration of the anti-proliferative agent of about 1 ng/g tissue to about 100 mg/g tissue. In some examples, the therapeutically effective dose may be sufficient to generate a tissue concentration of the anti-proliferative agent of about 10 ng/g tissue to about 100 mg/g tissue. In some examples, the therapeutically effective dose may be sufficient to generate a tissue concentration of the anti-proliferative agent of about 100 ng/g tissue to about 100 mg/g tissue.

In some examples, the therapeutic composition comprises sirolimus at a concentration sufficient to generate a tissue concentration of sirolimus in a sclera of the eye of within a range of about 0.1 ng/g tissue to about 100 mg/g tissue within about 1 day. In some examples, the therapeutic composition comprises sirolimus at a concentration sufficient to generate a tissue concentration of sirolimus in a sclera of the eye of within a range of about 0.1 ng/g tissue to about 100 mg/g tissue within about 72 days.

In some examples, the therapeutically effective dose is sufficient to maintain a tissue concentration of sirolimus of about 0.1 ng/g tissue to about 100 mg/g tissue for about 1 day to about 1 year, 30 days to about 1 year, 3 months to about 1 year, or 6 months to about 1 year.

In some examples, the anti-proliferative agent comprises novolimus and is present in the therapeutic composition at a concentration effective to achieve a tissue concentration of the anti-proliferative agent of about 0.1 ng/g tissue to about 100 mg/g tissue when a therapeutically effective dose of the therapeutic composition is delivered to the tissue. In some examples, the therapeutically effective dose may be sufficient to generate a tissue concentration of the anti-proliferative agent of about 0.2 ng/g tissue to about 100 mg/g tissue. In some examples, the therapeutically effective dose may be sufficient to generate a tissue concentration of the anti-proliferative agent of 0.5 ng/g tissue to about 100 mg/g tissue. In some examples, the therapeutically effective dose may be sufficient to generate a tissue concentration of the anti-proliferative agent of about 1 ng/g tissue to about 100 mg/g tissue. In some examples, the therapeutically effective dose may be sufficient to generate a tissue concentration of the anti-proliferative agent of about 10 ng/g tissue to about 100 mg/g tissue. In some examples, the therapeutically effective dose may be sufficient to generate a tissue concentration of the anti-proliferative agent of about 100 ng/g tissue to about 100 mg/g tissue.

In some examples, the therapeutic composition comprises novolimus at a concentration sufficient to generate a tissue concentration of sirolimus in a sclera of the eye of within a range of about 0.1 ng/g tissue to about 100 mg/g tissue within about 1 day. In some examples, the therapeutic composition comprises novolimus at a concentration sufficient to generate a tissue concentration of sirolimus in a sclera of the eye of within a range of about 0.1 ng/g tissue to about 100 mg/g tissue within about 72 days.

In some examples, the therapeutically effective dose is sufficient to maintain a tissue concentration of novolimus of about 0.1 ng/g tissue to about 100 mg/g tissue for about 1 day to about 1 year, 30 days to about 1 year, 3 months to about 1 year, or 6 months to about 1 year.

In some examples, the anti-proliferative agent comprises myolimus and is present in the therapeutic composition at a concentration effective to achieve a tissue concentration of the anti-proliferative agent of about 0.1 ng/g tissue to about 100 mg/g tissue when a therapeutically effective dose of the therapeutic composition is delivered to the tissue. In some examples, the therapeutically effective dose may be sufficient to generate a tissue concentration of the anti-proliferative agent of about 0.2 ng/g tissue to about 100 mg/g tissue. In some examples, the therapeutically effective dose may be sufficient to generate a tissue concentration of the anti-proliferative agent of 0.5 ng/g tissue to about 100 mg/g tissue. In some examples, the therapeutically effective dose may be sufficient to generate a tissue concentration of the anti-proliferative agent of about 1 ng/g tissue to about 100 mg/g tissue. In some examples, the therapeutically effective dose may be sufficient to generate a tissue concentration of the anti-proliferative agent of about 10 ng/g tissue to about 100 mg/g tissue. In some examples, the therapeutically effective dose may be sufficient to generate a tissue concentration of the anti-proliferative agent of about 100 ng/g tissue to about 100 mg/g tissue.

In some examples, the therapeutic composition comprises myolimus at a concentration sufficient to generate a tissue concentration of sirolimus in a sclera of the eye of within a range of about 0.1 ng/g tissue to about 100 mg/g tissue within about 1 day. In some examples, the therapeutic composition comprises myolimus at a concentration sufficient to generate a tissue concentration of sirolimus in a sclera of the eye of within a range of about 0.1 ng/g tissue to about 100 mg/g tissue within about 72 days.

In some examples, the therapeutically effective dose is sufficient to maintain a tissue concentration of myolimus of about 0.1 ng/g tissue to about 100 mg/g tissue for about 1 day to about 1 year, 30 days to about 1 year, 3 months to about 1 year, or 6 months to about 1 year.

In some examples, the anti-angiogenic agent comprises ranibizumab and is present in the therapeutic composition at a concentration effective to achieve a tissue concentration of the anti-angiogenic agent of about 0.1 ng/g tissue to about 100 mg/g tissue when a therapeutically effective dose of the therapeutic composition is delivered to the tissue. In some examples, the therapeutically effective dose may be sufficient to generate a tissue concentration of the anti-proliferative agent of about 0.2 ng/g tissue to about 100 mg/g tissue. In some examples, the therapeutically effective dose may be sufficient to generate a tissue concentration of the anti-proliferative agent of 0.5 ng/g tissue to about 100 mg/g tissue. In some examples, the therapeutically effective dose may be sufficient to generate a tissue concentration of the anti-proliferative agent of about 1 ng/g tissue to about 100 mg/g tissue. In some examples, the therapeutically effective dose may be sufficient to generate a tissue concentration of the anti-proliferative agent of about 10 ng/g tissue to about 100 mg/g tissue. In some examples, the therapeutically effective dose may be sufficient to generate a tissue concentration of the anti-proliferative agent of about 100 ng/g tissue to about 100 mg/g tissue.

In some examples, the therapeutically effective dose is sufficient to maintain a tissue concentration of ranibizumab of about 0.1 ng/g tissue to about 100 mg/g tissue for about 1 day to about 1 year, 30 days to about 1 year, 3 months to about 1 year, or 6 months to about 1 year.

In some examples, the therapeutically effective dose is sufficient to maintain a tissue concentration of brolucizumab within a range of about 0.1 ng/g tissue to about 100 mg/g tissue for about 1 day to about 1 year, 30 days to about 1 year, 3 months to about 1 year, or 6 months to about 1 year.In some examples, the anti-angiogenic agent comprises aflibercept and is present in the therapeutic composition at a concentration effective to achieve a tissue concentration of the anti-angiogenic agent of about 0.1 ng/g tissue to about 100 mg/g tissue when a therapeutically effective dose of the therapeutic composition is delivered to the tissue. In some examples, the therapeutically effective dose may be sufficient to generate a tissue concentration of the anti-proliferative agent of about 0.2 ng/g tissue to about 100 mg/g tissue. In some examples, the therapeutically effective dose may be sufficient to generate a tissue concentration of the anti-proliferative agent of 0.5 ng/g tissue to about 100 mg/g tissue. In some examples, the therapeutically effective dose may be sufficient to generate a tissue concentration of the anti-proliferative agent of about 1 ng/g tissue to about 100 mg/g tissue. In some examples, the therapeutically effective dose may be sufficient to generate a tissue concentration of the anti-proliferative agent of about 10 ng/g tissue to about 100 mg/g tissue. In some examples, the therapeutically effective dose may be sufficient to generate a tissue concentration of the anti-proliferative agent of about 100 ng/g tissue to about 100 mg/g tissue.

In some examples, the therapeutically effective dose is sufficient to maintain a tissue concentration of aflibercept of about 0.1 ng/g tissue to about 100 mg/g tissue for about 1 day to about 1 year, 30 days to about 1 year, 3 months to about 1 year, or 6 months to about 1 year.

In some examples, the anti-angiogenic agent comprises pegaptanib and is present in the therapeutic composition at a concentration effective to achieve a tissue concentration of the anti-angiogenic agent of about 0.1 ng/g tissue to about 100 mg/g tissue when a therapeutically effective dose of the therapeutic composition is delivered to the tissue. In some examples, the therapeutically effective dose may be sufficient to generate a tissue concentration of the anti-proliferative agent of about 0.2 ng/g tissue to about 100 mg/g tissue. In some examples, the therapeutically effective dose may be sufficient to generate a tissue concentration of the anti-proliferative agent of 0.5 ng/g tissue to about 100 mg/g tissue. In some examples, the therapeutically effective dose may be sufficient to generate a tissue concentration of the anti-proliferative agent of about 1 ng/g tissue to about 100 mg/g tissue. In some examples, the therapeutically effective dose may be sufficient to generate a tissue concentration of the anti-proliferative agent of about 10 ng/g tissue to about 100 mg/g tissue. In some examples, the therapeutically effective dose may be sufficient to generate a tissue concentration of the anti-proliferative agent of about 100 ng/g tissue to about 100 mg/g tissue.

In some examples, the therapeutically effective dose is sufficient to maintain a tissue concentration of pegaptanib of about 0.1 ng/g tissue to about 100 mg/g tissue for about 1 day to about 1 year, 30 days to about 1 year, 3 months to about 1 year, or 6 months to about 1 year.

In some examples, a therapeutically effective dose of the therapeutic composition may be delivered to the target tissue to generate a therapeutically effective concentration in the target tissue. The therapeutic composition may be formulated such that the therapeutically effective concentration may be substantially higher than a systemic blood concentration of each agent in order to treat effectively the disease or condition of the lung. Preferably, the tissue concentration is at least about 1 times a median maximum serum concentration (Cₘₐₓ) blood concentration, at least 1.5 times, 2 times, or 5 times higher than the systemic Cₘₐₓ blood concentration or about 10 times higher than the systemic dose.

In some examples, formulations of the present invention for treating dry eye may include drugs to reduce eyelid inflammation, antibiotics; substances to control cornea inflammation, for example containing immune-suppressing medication such as cyclosporine, corticosteroids, and the like, tear-stimulating drugs such as cholinergic agents (e.g., pilocarpine, cevimeline), and the like.

In some examples, formulations for glaucoma treatment may include prostaglandin analogs, such as bimatoprost, latanoprost, tafluprost, travoprost and the like; beta blockers, such as betaxolol, timolol and the like; alpha-adrenergic agonists, such as apraclonidine, brimonidine and the like; carbonic anhydrase inhibitors, such as brinzolamide, dorzolamide, cholinergic agents, such as pilocarpine and cevimeline; and the like, as well as combinations, such as timolol and dorzolamide, brimonidine and timolol, brimonidine and brinzolamide, and the like.

In some examples, a therapeutically effective concentration of the therapeutic composition may be delivered to the target tissue. The therapeutic composition may be formulated such that the therapeutically effective concentration may be substantially higher than a systemic therapeutic blood concentration of each agent, preferably higher than a systemic therapeutic blood Cₘₐₓ dose of each agent, while the agent blood Cₘₐₓ (from agent diffusing into the systemic circulation) remains under systemic dose of the agent blood Cₘₐₓ. For example, tissue concentration of the agent Apixaban in the ocular tissue (e.g.,the scleral tissue) may range from 0.1 ng/g tissue while the blood concentration in the systemic circulation remains below 100 ng/ml of blood.

For example, typical blood concentrations for systemically delivered apixaban ranges from about 10 ng/ml to about 40 ng/ml in the blood and the Cₘₐₓ depending on the doses ranges from about 50 ng/ml to about 100 ng/ml (sometimes up to about 200 ng/ml). If we assume density of the tissue (being mostly water) is about 1 g/ml, these ranges can be converted from ng/ml to ng/g, with 10 ng/ml being equivalent to about 10 ng/g tissue and 200 ng/ml being equivalent to about 200 ng/g tissue, which means Cₘₐₓ is at or below 200 ng/g of tissue.

In some examples, the therapeutically effective dose in the ocular tissue may be sufficient to generate a blood concentration of the direct factor Xa inhibitor which is less than about 200 ng/ml, 100 ng/ml, 50 ng/ml, 40 ng/ml, or 10 ng/ml.

In some examples, the therapeutically effective dose may be sufficient to generate a blood concentration of the direct factor Xa inhibitor which is smaller than a median maximum serum concentration (Cₘₐₓ) of the direct factor Xa inhibitor generated by systemic delivery of the direct factor Xa inhibitor to achieve the same tissue concentration at the site of the inflammatory, fibrotic, and/or clot formation ophthalmic disease or condition. In some examples, the therapeutically effective dose may be sufficient to generate a blood concentration of the direct factor Xa inhibitor which does not exceed a median maximum serum concentration (Cₘₐₓ) of the direct factor Xa inhibitor generated by systemic delivery of the direct factor Xa inhibitor to achieve the same tissue concentration at the site of the inflammatory, fibrotic, and/or clot formation ophthalmic disease or condition for more than about 6 hours to about 3 days. In some examples, the systemic delivery comprises a single oral dose, a daily oral dose, or a smallest oral dose of the direct factor Xa inhibitor. In some examples, the Cₘₐₓ is measured using one of plasma blood, serum blood, or whole blood. In other examples, the median Cₘₐₓ is 80 ng/ml, or 123 ng/ml, or 171 ng/ml, or 321 ng/ml, or 480 ng/ml of blood.

In some examples, the therapeutically effective dose may be sufficient to generate a blood concentration of the direct factor Xa inhibitor which is less than a systemic therapeutic concentration of the direct factor Xa inhibitor for any systemic indication.

In some examples, the therapeutic composition is formulated to release a dose of the direct factor Xa inhibitor sufficient to generate a plasma drug level area under the curve (AUC (0-24) or AUC (0-∞)) in ng.h/ml which is smaller than a median (AUC (0-24) or AUC (0-∞)) in ng.h/ml of the direct factor Xa inhibitor generated by systemic delivery of the direct factor Xa inhibitor to achieve the same tissue concentration at the site of the inflammatory, fibrotic, and/or clot formation ophthalmic disease or condition. In other examples, the therapeutic composition is formulated to release a dose of the direct factor Xa inhibitor sufficient to generate a plasma drug level area under the curve (AUC (0-24) or AUC (0-∞)) in ng.h/ml which is smaller than a median (AUC (0-24) or AUC (0-∞)) in ng.h/ml of the direct factor Xa inhibitor generated by systemic delivery of the direct factor Xa inhibitor when taking one or more oral dose of said factor Xa inhibitor. In some examples, the systemic delivery comprises a single oral dose, a daily oral dose, or a smallest oral dose of the direct factor Xa inhibitor. In some examples, the (AUC (0-24) or AUC (0-∞)) is measured using one of plasma blood, serum blood, or whole blood. In other examples, the median (AUC (0-24) or AUC (0-∞)) is 724 ng.h/ml, or1437 ng.h/ml, or 2000 ng.h/ml, or 4000 ng.h/ml.

In some examples, the therapeutically effective dose in ocular tissue may be sufficient to generate a blood concentration of the direct factor IIa inhibitor which is less than about 200 ng/ml, 100 ng/ml, 50 ng/ml, 40 ng/ml, or 10 ng/ml.

In some examples, the therapeutically effective dose may be sufficient to generate a blood concentration of the direct factor IIa inhibitor which is smaller than a median maximum serum concentration (Cₘₐₓ) of the direct factor IIa inhibitor generated by systemic delivery of the direct factor IIa inhibitor to achieve the same tissue concentration at the site of the the inflammatory, fibrotic, and/or clot formation ophthalmic disease or condition. In some examples, the therapeutically effective dose may be sufficient to generate a blood concentration of the direct factor IIa inhibitor which does not exceed a median maximum serum concentration (Cₘₐₓ) of the direct factor IIa inhibitor generated by systemic delivery of the direct factor IIa inhibitor to achieve the same tissue concentration at the site of the inflammatory, fibrotic, and/or clot formation ophthalmic disease or condition for more than about 6 hours to about 3 days. In some examples, the systemic delivery comprises a single oral dose, a daily oral dose, or a smallest oral dose of the direct factor IIa inhibitor. In some examples, the systemic delivery comprises a single oral dose, a daily oral dose, or a smallest oral dose of the direct factor IIa inhibitor. In some examples, the Cₘₐₓ is measured using one of plasma blood, serum blood, or whole blood. In other examples, the median Cₘₐₓ is 80 ng/ml, or 123 ng/ml, or 171 ng/ml, or 321 ng/ml, or 480 ng/ml of blood.

In some examples, the therapeutic composition is formulated to release a dose of the direct factor IIa inhibitor sufficient to generate a plasma drug level area under the curve (AUC (0-24) or AUC (0-∞)) in ng.h/ml which is smaller than a median (AUC (0-24) or AUC (0-∞)) in ng.h/ml of the direct factor IIa inhibitor generated by systemic delivery of the direct factor IIa inhibitor to achieve the same tissue concentration at the site of the inflammatory, fibrotic, and/or clot formation ophthalmic disease or condition. In other examples, the therapeutic composition is formulated to release a dose of the direct factor IIa inhibitor sufficient to generate a plasma drug level area under the curve (AUC (0-24) or AUC (0-∞)) in ng.h/ml which is smaller than a median (AUC (0-24) or AUC (0-∞)) in ng.h/ml of the direct factor IIa inhibitor generated by systemic delivery of the direct factor IIa inhibitor when taking one or more oral dose of said factor IIa inhibitor. In some examples, the systemic delivery comprises a single oral dose, a daily oral dose, or a smallest oral dose of the direct factor IIa inhibitor. In some examples, the (AUC (0-24) or AUC (0-∞)) is measured using one of plasma blood, serum blood, or whole blood. In other examples, the median (AUC (0-24) or AUC (0-∞)) is 724 ng.h/ml, or1437 ng.h/ml, or 2000 ng.h/ml, or 4000 ng.h/ml.

In some examples, the therapeutically effective dose in ocular tissue may be sufficient to generate a blood concentration of the anti-fibrotic agent comprising nintedanib which is less than about 100 ng/ml, 50 ng/ml, 40 ng/ml, or 30 ng/ml.

In some examples, the therapeutically effective dose may be sufficient to generate a blood concentration of nintedanib which is smaller than a median maximum serum concentration (Cₘₐₓ) of nintedanib generated by systemic delivery of nintedanib to achieve the same tissue concentration at the site of the inflammatory, fibrotic, and/or clot formation ophthalmic disease or condition. In some examples, the therapeutically effective dose may be sufficient to generate a blood concentration of nintedanib which does not exceed a median maximum serum concentration (Cₘₐₓ) of nintedanib generated by systemic delivery of nintedanib to achieve the same tissue concentration at the site of the inflammatory, fibrotic, and/or clot formation ophthalmic disease or condition for more than about 6 hours to about 12 hours. In some examples, the systemic delivery comprises a single oral dose, a daily oral dose, or a smallest oral dose of nintedanib. In some examples, the systemic delivery comprises a single oral dose, a daily oral dose, or a smallest oral dose of nintedanib. In some examples, the Cₘₐₓ is measured using one of plasma blood, serum blood, or whole blood. In other examples, the median Cₘₐₓ is 31.8 ng/ml or 43.2 ng/ml of blood.

In some examples, the therapeutic composition is formulated to release a dose of nintedanib sufficient to generate a plasma drug level area under the curve (AUC (0-24) or AUC (0-∞)) in ng.h/ml which is smaller than a median (AUC (0-24) or AUC (0-∞)) in ng.h/ml of nintedanib generated by systemic delivery of nintedanib to achieve the same tissue concentration at the site of the inflammatory, fibrotic, and/or clot formation ophthalmic disease or condition. In other examples, the therapeutic composition is formulated to release a dose of nintedanib sufficient to generate a plasma drug level area under the curve (AUC (0-24) or AUC (0-∞)) in ng.h/ml which is smaller than a median (AUC (0-24) or AUC (0-∞)) in ng.h/ml of nintedanib generated by systemic delivery of nintedanib when taking one or more oral dose of nintedanib. In some examples, the systemic delivery comprises a single oral dose, a daily oral dose, or a smallest oral dose of nintedanib. In some examples, the (AUC (0-24) or AUC (0-∞)) is measured using one of plasma blood, serum blood, or whole blood. In other examples, the median (AUC (0-24) or AUC (0-∞)) is 266 ng.h/ml.

In some examples, the therapeutically effective dose in ocular tissue may be sufficient to generate a blood concentration of the anti-fibrotic agent comprising pirfenidone which is less than about 20000 ng/ml, 12000 ng/ml, 7000 ng/ml, or 6500 ng/ml.

In some examples, the therapeutically effective dose may be sufficient to generate a blood concentration of pirfenidone which is smaller than a median maximum serum concentration (Cₘₐₓ) of pirfenidone generated by systemic delivery of pirfenidone to achieve the same tissue concentration at the site of the inflammatory, fibrotic, and/or clot formation ophthalmic disease or condition. In some examples, the therapeutically effective dose may be sufficient to generate a blood concentration of pirfenidone which does not exceed a median maximum serum concentration (Cₘₐₓ) of pirfenidone generated by systemic delivery of pirfenidone to achieve the same tissue concentration at the site of the inflammatory, fibrotic, and/or clot formation ophhtalmic disease or condition for more than about 6 hours to about 3 days. In some examples, the systemic delivery comprises a single oral dose, a daily oral dose, or a smallest oral dose of pirfenidone. In some examples, the systemic delivery comprises a single oral dose, a daily oral dose, or a smallest oral dose of pirfenidone. In some examples, the Cₘₐₓ is measured using one of plasma blood, serum blood, or whole blood. In other examples, the median Cₘₐₓ is 6560 ng/ml, or 7640 ng/ml, or 12300 ng/ml, or 12500 ng/ml, or 12600 ng/ml, or 19800 ng/ml of blood.

In some examples, the therapeutic composition is formulated to release a dose of pirfenidone sufficient to generate a plasma drug level area under the curve (AUC (0-24) or AUC (0-∞)) in ng.h/ml which is smaller than a median (AUC (0-24) or AUC (0-∞)) in ng.h/ml of pirfenidone generated by systemic delivery of pirfenidone to achieve the same tissue concentration at the site of the inflammatory, fibrotic, and/or clot formation ophthalmic disease or condition. In other examples, the therapeutic composition is formulated to release a dose of pirfenidone sufficient to generate a plasma drug level area under the curve (AUC (0-24) or AUC (0-∞)) in ng.h/ml which is smaller than a median (AUC (0-24) or AUC (0-∞)) in ng.h/ml of pirfenidone generated by systemic delivery of pirfenidone when taking one or more oral dose of pirfenidone. In some examples, the systemic delivery comprises a single oral dose, a daily oral dose, or a smallest oral dose of pirfenidone. In some examples, the (AUC (0-24) or AUC (0-∞)) is measured using one of plasma blood, serum blood, or whole blood. In other examples, the median (AUC (0-24) or AUC (0-∞)) is 39800 ng.h/ml, or 40900 ng.h/ml, or 49400 ng.h/ml, or 49700 ng.h/ml, or 55900 ng.h/ml, or 92900 ng.h/ml.

In some examples, the therapeutically effective dose in ocular tissue may be sufficient to generate a blood concentration of the anti-viral/anti-diabetic agent comprising metformin which is less than about 1800 µg/ml, 900 µg/ml, 500 µg/ml, 50 µg/ml, 1.3 µg/ml, 1 µg/ml, or 0.8 µg/ml.

In some examples, the therapeutically effective dose may be sufficient to generate a blood concentration of metformin which is smaller than a median maximum serum concentration (Cₘₐₓ) of metformin generated by systemic delivery of metformin to achieve the same tissue concentration at the site of the inflammatory, fibrotic, and/or clot formation ophthalmic disease or condition. In some examples, the therapeutically effective dose may be sufficient to generate a blood concentration of metformin which does not exceed a median maximum serum concentration (Cₘₐₓ) of metformin generated by systemic delivery of metformin to achieve the same tissue concentration at the site of the inflammatory, fibrotic, and/or clot formation ophthalmic disease or condition for more than about 6 hours to about 3 days. In some examples, the systemic delivery comprises a single oral dose, a daily oral dose, or a smallest oral dose of metformin. In some examples, the systemic delivery comprises a single oral dose, a daily oral dose, or a smallest oral dose of metformin. In some examples, the Cₘₐₓ is measured using one of plasma blood, serum blood, or whole blood. In other examples, the median Cₘₐₓ is 811.9 ng/ml, or 959.1 ng/ml, or 1301.4 ng/ml of blood.

In some examples, the therapeutic composition is formulated to release a dose of metformin sufficient to generate a plasma drug level area under the curve (AUC (0-24) or AUC (0-∞)) in ng.h/ml which is smaller than a median (AUC (0-24) or AUC (0-∞)) in ng.h/ml of metformin generated by systemic delivery of metformin to achieve the same tissue concentration at the site of the inflammatory, fibrotic, and/or clot formation ophthalmic disease or condition. In other examples, the therapeutic composition is formulated to release a dose of metformin sufficient to generate a plasma drug level area under the curve (AUC (0-24) or AUC (0-∞)) in ng.h/ml which is smaller than a median (AUC (0-24) or AUC (0-∞)) in ng.h/ml of metformin generated by systemic delivery of metformin when taking one or more oral dose of metformin. In some examples, the systemic delivery comprises a single oral dose, a daily oral dose, or a smallest oral dose of metformin. In some examples, the (AUC (0-24) or AUC (0-∞)) is measured using one of plasma blood, serum blood, or whole blood. In other examples, the median (AUC (0-24) or AUC (0-∞)) is 14182 ng.h/ml, or 15260 ng.h/ml, or 15342 ng.h/ml.

In some examples, the therapeutically effective dose in ocular tissue may be sufficient to generate a blood concentration of the anti-proliferative agent comprising sirolimus which is less than about 200 ng/ml.

In some examples, the therapeutically effective dose may be sufficient to generate a blood concentration of sirolimus which is smaller than a median maximum serum concentration (Cₘₐₓ) of sirolimus generated by systemic delivery of sirolimus to achieve the same tissue concentration at the site of the inflammatory, fibrotic, and/or clot formation ophthalmic disease or condition. In some examples, the therapeutically effective dose may be sufficient to generate a blood concentration of sirolimus which does not exceed a median maximum serum concentration (Cₘₐₓ) of sirolimus generated by systemic delivery of sirolimus to achieve the same tissue concentration at the site of the inflammatory, fibrotic, and/or clot formation ophthalmic disease or condition for more than about 6 hours to about 7 days. In some examples, the systemic delivery comprises a single oral dose, a daily oral dose, or a smallest oral dose of sirolimus. In some examples, the systemic delivery comprises a single oral dose, a daily oral dose, or a smallest oral dose of sirolimus. In some examples, the Cₘₐₓ is measured using one of plasma blood, serum blood, or whole blood. In other examples, the median Cₘₐₓ is less than about 144 ng/ml.

In some examples, the therapeutic composition is formulated to release a dose of sirolimus sufficient to generate a plasma drug level area under the curve (AUC (0-24) or AUC (0-∞)) in ng.h/ml which is smaller than a median (AUC (0-24) or AUC (0-∞)) in ng.h/ml of sirolimus generated by systemic delivery of sirolimus to achieve the same tissue concentration at the site of the inflammatory, fibrotic, and/or clot formation ophthalmic disease or condition. In other examples, the therapeutic composition is formulated to release a dose of sirolimus sufficient to generate a plasma drug level area under the curve (AUC (0-24) or AUC (0-∞)) in ng.h/ml which is smaller than a median (AUC (0-24) or AUC (0-∞)) in ng.h/ml of sirolimus generated by systemic delivery of sirolimus when taking one or more oral dose of sirolimus. In some examples, the systemic delivery comprises a single oral dose, a daily oral dose, or a smallest oral dose of sirolimus. In some examples, the (AUC (0-24) or AUC (0-∞)) is measured using one of plasma blood, serum blood, or whole blood. In other examples, the median (AUC (0-24) or AUC (0-∞)) is 160 ng.h/ml, or 200 ng.h/ml, or 230 ng.h/ml, or 400 ng.h/ml, or 5432 ng.h/ml, or 34920 ng.h/ml.

In some examples, the therapeutically effective dose in ocular tissue may be sufficient to generate a blood concentration of the anti-proliferative agent comprising novolimus which is less than about 100 ng/ml.

In some examples, the therapeutically effective dose may be sufficient to generate a blood concentration of novolimus which is smaller than a median maximum serum concentration (Cₘₐₓ) of novolimus generated by systemic delivery of novolimus to achieve the same tissue concentration at the site of the inflammatory, fibrotic, and/or clot formation ophthalmic disease or condition. In some examples, the therapeutically effective dose may be sufficient to generate a blood concentration of novolimus which does not exceed a median maximum serum concentration (Cₘₐₓ) of novolimus generated by systemic delivery of novolimus to achieve the same tissue concentration at the site of the inflammatory, fibrotic, and/or clot formation ophthalmic disease or condition for more than about 6 hours to about 7 days. In some examples, the systemic delivery comprises a single oral dose, a daily oral dose, or a smallest oral dose of novolimus. In some examples, the systemic delivery comprises a single oral dose, a daily oral dose, or a smallest oral dose of novolimus. In some examples, the Cₘₐₓ is measured using one of plasma blood, serum blood, or whole blood. In other examples, the median Cₘₐₓ is less than about 100 ng/ml.

In some examples, the therapeutic composition is formulated to release a dose of novolimus sufficient to generate a plasma drug level area under the curve (AUC (0-24) or AUC (0-∞)) in ng.h/ml which is smaller than a median (AUC (0-24) or AUC (0-∞)) in ng.h/ml of novolimus generated by systemic delivery of novolimus to achieve the same tissue concentration at the site of the inflammatory, fibrotic, and/or clot formation ophthalmic disease or condition. In other examples, the therapeutic composition is formulated to release a dose of novolimus sufficient to generate a plasma drug level area under the curve (AUC (0-24) or AUC (0-∞)) in ng.h/ml which is smaller than a median (AUC (0-24) or AUC (0-∞)) in ng.h/ml of novolimus generated by systemic delivery of novolimus when taking one or more oral dose of novolimus. In some examples, the systemic delivery comprises a single oral dose, a daily oral dose, or a smallest oral dose of novolimus. In some examples, the (AUC (0-24) or AUC (0-∞)) is measured using one of plasma blood, serum blood, or whole blood. In other examples, the median (AUC (0-24) or AUC (0-∞)) is 61 ng.h/ml, or 489 ng.h/ml to 1032 ng.h/ml, or within a range of about 3060 ng.h/ml to about 6660 ng.h/ml.

In some examples, the therapeutically effective dose in ocular tissue may be sufficient to generate a blood concentration of the anti-proliferative agent comprising myolimus which is less than about 200 ng/ml.

In some examples, the therapeutically effective dose may be sufficient to generate a blood concentration of myolimus which is smaller than a median maximum serum concentration (Cₘₐₓ) of myolimus generated by systemic delivery of myolimus to achieve the same tissue concentration at the site of the inflammatory, fibrotic, and/or clot formation ophthalmic disease or condition. In some examples, the therapeutically effective dose may be sufficient to generate a blood concentration of myolimus which does not exceed a median maximum serum concentration (Cₘₐₓ) of myolimus generated by systemic delivery of myolimus to achieve the same tissue concentration at the site of the inflammatory, fibrotic, and/or clot formation ophthalmic disease or condition for more than about 6 hours to about 7 days. In some examples, the systemic delivery comprises a single oral dose, a daily oral dose, or a smallest oral dose of myolimus. In some examples, the systemic delivery comprises a single oral dose, a daily oral dose, or a smallest oral dose of myolimus. In some examples, the Cₘₐₓ is measured using one of plasma blood, serum blood, or whole blood. In other examples, the median Cₘₐₓ is less than about 200 ng/ml.

In some examples, the therapeutic composition is formulated to release a dose of myolimus sufficient to generate a plasma drug level area under the curve (AUC (0-24) or AUC (0-∞)) in ng.h/ml which is smaller than a median (AUC (0-24) or AUC (0-∞)) in ng.h/ml of myolimus generated by systemic delivery of myolimus to achieve the same tissue concentration at the site of the inflammatory, fibrotic, and/or clot formation ophthalmic disease or condition. In other examples, the therapeutic composition is formulated to release a dose of myolimus sufficient to generate a plasma drug level area under the curve (AUC (0-24) or AUC (0-∞)) in ng.h/ml which is smaller than a median (AUC (0-24) or AUC (0-∞)) in ng.h/ml of myolimus generated by systemic delivery of myolimus when taking one or more oral dose of myolimus. In some examples, the systemic delivery comprises a single oral dose, a daily oral dose, or a smallest oral dose of myolimus. In some examples, the (AUC (0-24) or AUC (0-∞)) is measured using one of plasma blood, serum blood, or whole blood. In other examples, the median (AUC (0-24) or AUC (0-∞)) is 100 ng.h/ml, or 200 ng.h/ml, or 300 ng.h/ml, or 400 ng.h/ml, 1000 ng.h/ml or 5000 ng.h/ml, or 50000 ng.h/ml.

In some examples, the therapeutically effective dose in ocular tissue may be sufficient to generate a blood concentration of the anti-angiogenic agent comprising ranibizumab which is less than about 1000 µg/ml, 500 µg/ml, 250 µg/ml, 100 µg/ml or 50 µg/ml.,

In some examples, the therapeutically effective dose may be sufficient to generate a blood concentration of ranibizumab which is smaller than a median maximum serum concentration (Cₘₐₓ) of ranibizumab generated by systemic delivery of ranibizumab to achieve the same tissue concentration at the site of the inflammatory, fibrotic, and/or clot formation ophthalmic disease or condition. In some examples, the therapeutically effective dose may be sufficient to generate a blood concentration of ranibizumab which does not exceed a median maximum serum concentration (Cₘₐₓ) of ranibizumab generated by systemic delivery of ranibizumab to achieve the same tissue concentration at the site of the inflammatory, fibrotic, and/or clot formation ophthalmic disease or condition for more than about 6 hours to about 3 days. In some examples, the systemic delivery comprises a single oral dose, a daily oral dose, or a smallest oral dose of ranibizumab. In some examples, the systemic delivery comprises a single oral dose, a daily oral dose, or a smallest oral dose of ranibizumab. In some examples, the Cₘₐₓ is measured using one of plasma blood, serum blood, or whole blood. In other examples, the median Cₘₐₓ is 620 µg/ml, or 500 µg/ml, or 230 µg/ml, or 170 µg/ml or 120 µg/ml.

In some examples, the therapeutic composition is formulated to release a dose of ranibizumab sufficient to generate a plasma drug level area under the curve (AUC (0-24) or AUC (0-∞)) in ng.h/ml which is smaller than a median (AUC (0-24) or AUC (0-∞)) in ng.h/ml of ranibizumab generated by systemic delivery of ranibizumab to achieve the same tissue concentration at the site of the inflammatory, fibrotic, and/or clot formation ophthalmic disease or condition. In other examples, the therapeutic composition is formulated to release a dose of ranibizumab sufficient to generate a plasma drug level area under the curve (AUC (0-24) or AUC (0-∞)) in ng.h/ml which is smaller than a median (AUC (0-24) or AUC (0-∞)) in ng.h/ml of ranibizumab generated by systemic delivery of ranibizumab when taking one or more oral dose of ranibizumab. In some examples, the systemic delivery comprises a single oral dose, a daily oral dose, or a smallest oral dose of ranibizumab. In some examples, the (AUC (0-24) or AUC (0-∞)) is measured using one of plasma blood, serum blood, or whole blood. In other examples, the median (AUC (0-24) or AUC (0-∞)) is 0.2 µg.d/ml, or 1 µg.d/ml, or 10 µg.d/ml, or 150 µg.d/ml, or 220 µg.d/ml, or 315 µg.d/ml, or 433 µg.d/ml, or 586 µg.d/ml, or 687 µg.d/ml, or 1500 µg.d/ml, or 3230 µg.d/ml.

In some examples, the therapeutically effective dose in ocular tissue may be sufficient to generate a blood concentration of the anti-angiogenic agent comprising brolucizumab which is less than about 100 µg/ml.

In some examples, the therapeutically effective dose may be sufficient to generate a blood concentration of brolucizumab which is smaller than a median maximum serum concentration (Cₘₐₓ) of brolucizumab generated by systemic delivery of brolucizumab to achieve the same tissue concentration at the site of the inflammatory, fibrotic, and/or clot formation ophthalmic disease or condition. In some examples, the therapeutically effective dose may be sufficient to generate a blood concentration of brolucizumab which does not exceed a median maximum serum concentration (Cₘₐₓ) of brolucizumab generated by systemic delivery of brolucizumab to achieve the same tissue concentration at the site of the inflammatory, fibrotic, and/or clot formation ophthalmic disease or condition for more than about 6 hours to about 3 days. In some examples, the systemic delivery comprises a single oral dose, a daily oral dose, or a smallest oral dose of brolucizumab. In some examples, the systemic delivery comprises a single oral dose, a daily oral dose, or a smallest oral dose of brolucizumab. In some examples, the Cₘₐₓ is measured using one of plasma blood, serum blood, or whole blood. In other examples, the median Cₘₐₓ is 100 µg/ml, or 50 µg/ml, or 10 µg/ml,or 1 µg/ml or 0.1 µg/ml.

In some examples, the therapeutic composition is formulated to release a dose of brolucizumab sufficient to generate a plasma drug level area under the curve (AUC (0-24) or AUC (0-∞)) in ng.h/ml which is smaller than a median (AUC (0-24) or AUC (0-∞)) in ng.h/ml of brolucizumab generated by systemic delivery of brolucizumab to achieve the same tissue concentration at the site of the inflammatory, fibrotic, and/or clot formation ophthalmic disease or condition. In other examples, the therapeutic composition is formulated to release a dose of brolucizumab sufficient to generate a plasma drug level area under the curve (AUC (0-24) or AUC (0-∞)) in ng.h/ml which is smaller than a median (AUC (0-24) or AUC (0-∞)) in ng.h/ml of brolucizumab generated by systemic delivery of brolucizumab when taking one or more oral dose of brolucizumab. In some examples, the systemic delivery comprises a single oral dose, a daily oral dose, or a smallest oral dose of brolucizumab. In some examples, the (AUC (0-24) or AUC (0-∞)) is measured using one of plasma blood, serum blood, or whole blood. In other examples, the median (AUC (0-24) or AUC (0-∞)) is 0.2 µg.d/ml, or 1 µg.d/ml, or 10 µg.d/ml, or 30 µg.d/ml, or 60 µg.d/ml, or 100 µg.d/ml, or 200 µg.d/ml, or 300 µg.d/ml, or 400 µg.d/ml, or 500 µg.d/ml, or 700 µg.d/ml, or 1500 µg.d/ml.

In some examples, the therapeutically effective dose in ocular tissue may be sufficient to generate a blood concentration of the anti-angiogenic agent comprising aflibercept which is less than about 400 µg/ml.

In some examples, the therapeutically effective dose may be sufficient to generate a blood concentration of aflibercept which is smaller than a median maximum serum concentration (Cₘₐₓ) of aflibercept generated by systemic delivery of aflibercept to achieve the same tissue concentration at the site of the inflammatory, fibrotic, and/or clot formation ophthalmic disease or condition. In some examples, the therapeutically effective dose may be sufficient to generate a blood concentration of aflibercept which does not exceed a median maximum serum concentration (Cₘₐₓ) of aflibercept generated by systemic delivery of aflibercept to achieve the same tissue concentration at the site of the inflammatory, fibrotic, and/or clot formation ophthalmic disease or condition for more than about 6 hours to about 3 days. In some examples, the systemic delivery comprises a single oral dose, a daily oral dose, or a smallest oral dose of aflibercept. In some examples, the systemic delivery comprises a single oral dose, a daily oral dose, or a smallest oral dose of aflibercept. In some examples, the Cₘₐₓ is measured using one of plasma blood, serum blood, or whole blood. In other examples, the median Cₘₐₓ is 68 µg/ml or 74 µg/ml.

In some examples, the therapeutic composition is formulated to release a dose of aflibercept sufficient to generate a plasma drug level area under the curve (AUC (0-24) or AUC (0-∞)) in ng.h/ml which is smaller than a median (AUC (0-24) or AUC (0-∞)) in ng.h/ml of aflibercept generated by systemic delivery of aflibercept to achieve the same tissue concentration at the site of the inflammatory, fibrotic, and/or clot formation ophthalmic disease or condition. In other examples, the therapeutic composition is formulated to release a dose of aflibercept sufficient to generate a plasma drug level area under the curve (AUC (0-24) or AUC (0-∞)) in ng.h/ml which is smaller than a median (AUC (0-24) or AUC (0-∞)) in ng.h/ml of aflibercept generated by systemic delivery of aflibercept when taking one or more oral dose of aflibercept. In some examples, the systemic delivery comprises a single oral dose, a daily oral dose, or a smallest oral dose of aflibercept. In some examples, the (AUC (0-24) or AUC (0-∞)) is measured using one of plasma blood, serum blood, or whole blood. In other examples, the median (AUC (0-24) or AUC (0-∞)) is 124 µg.d/ml or 174 µg.d/ml.

In some examples, the therapeutically effective dose in ocular tissue may be sufficient to generate a blood concentration of the anti-angiogenic agent comprising pegaptanib which is less than about 1000 µg/ml.

In some examples, the therapeutically effective dose may be sufficient to generate a blood concentration of pegaptanib which is smaller than a median maximum serum concentration (Cₘₐₓ) of pegaptanib generated by systemic delivery of pegaptanib to achieve the same tissue concentration at the site of the inflammatory, fibrotic, and/or clot formation ophthalmic disease or condition. In some examples, the therapeutically effective dose may be sufficient to generate a blood concentration of pegaptanib which does not exceed a median maximum serum concentration (Cₘₐₓ) of pegaptanib generated by systemic delivery of pegaptanib to achieve the same tissue concentration at the site of the inflammatory, fibrotic, and/or clot formation ophthalmic disease or condition for more than about 6 hours to about 3 days. In some examples, the systemic delivery comprises a single oral dose, a daily oral dose, or a smallest oral dose of pegaptanib In some examples, the systemic delivery comprises a single oral dose, a daily oral dose, or a smallest oral dose of pegaptanib. In some examples, the Cₘₐₓ is measured using one of plasma blood, serum blood, or whole blood. In other examples, the median Cₘₐₓ is 0.1 µg/ml to 2 µg/ml.

In some examples, the therapeutic composition is formulated to release a dose of pegaptanib sufficient to generate a plasma drug level area under the curve (AUC (0-24) or AUC (0-∞)) in ng.h/ml which is smaller than a median (AUC (0-24) or AUC (0-∞)) in ng.h/ml of pegaptanib generated by systemic delivery of pegaptanib to achieve the same tissue concentration at the site of the inflammatory, fibrotic, and/or clot formation ophthalmic disease or condition. In other examples, the therapeutic composition is formulated to release a dose of pegaptanib sufficient to generate a plasma drug level area under the curve (AUC (0-24) or AUC (0-∞)) in ng.h/ml which is smaller than a median (AUC (0-24) or AUC (0-∞)) in ng.h/ml of pegaptanib generated by systemic delivery of pegaptanib when taking one or more oral dose of pegaptanib. In some examples, the systemic delivery comprises a single oral dose, a daily oral dose, or a smallest oral dose of pegaptanib. In some examples, the (AUC (0-24) or AUC (0-∞)) is measured using one of plasma blood, serum blood, or whole blood. In other examples, the median (AUC (0-24) or AUC (0-∞)) is 2 µg.h/ml, or 8 µg.h/ml to 9 µg.h/ml, or 26 µg.h/ml to 29 µg.h/ml.

In some examples, measurements of blood or tissue described herein comprise one or more of mammalian blood or tissue, porcine blood or tissue, human blood or tissue, rabbit blood or tissue, rat blood or tissue, mouse blood or tissue, or the like.

In some examples, measurements of dose described herein are of human and adjustment to dose to account for total blood and/or fluid in other animal species compared to human may be necessary to equate to human dose.

In some examples, one or more agents in the therapeutic composition may have low solubility. For example, one or more of the direct factor Xa inhibitor, the direct factor IIa inhibitor, the anti-fibrotic agent, the anti-angiogenic agent, the anti-proliferative agent, the anti-viral/anti-diabetic agent, etc. may have low solubility.

In some examples, two or more active substances (e.g., direct factor Xa inhibitor, direct factor IIa inhibitor, anti-proliferative agent, anti-viral agent, etc.) may be present in a polymeric material. In some examples, the polymeric material may comprise a material selected from the group consisting of polyacrylates, polymethacrylates, poly(n-butyl methacrylate), poly(hydroxyethylmethacrylate), poly(ethylene glycol), polyethylene oxide (PEO), polydimethylsiloxane, polyvinylpyrrolidone, ethylene-vinyl acetate, phosphorylcholine-containing polymers, poly(2-methacryloyloxyethylphosphorylcholine), poly(2-methacryloyloxyethylphosphorylcholine-co-butyl methacrylate), and copolymers and combinations thereof.

In some examples, implants or vehicles for ocular drug delivery includes but are not limited to ocular inserts, microneedles with hollow for injection, drug coated microneedles with biodegradable or erode polymers, drug coated contact lens, intraocular lens implant with drug, a Port Delivery System (PDS) implant, and the like.

In some examples, an implant or vehicles delivery one or more drugs locally, wherein locally comprises delivery of said one or more drugs to one or more of site-specific ocular location.

In some examples, the therapeutic composition is formulated to release substantially all of the one or more active substances and the combination had synergistic effects, the effects that was better than either alone. In some examples, the therapeutic composition may be delivered simultaneously or sequentially in any order or combination as desired with any combination of methods or devices desired. For example, the microneedle can be configured to deposit a drug formulation within the cornea or sclera to release substantially all of the one or more active substances to one or more ocular tissues surrounding the depot to generate a tissue concentration of each of the agents at the treated site within about certain time of the ocular tissue contact. This release could be extended or sustained than that obtained by eye drops or other topical applications. The microneedle can be configured to delivery liquid formulation or can be configured as an implant with a degradable polymer and drugs.

One or more microneedles can be employed to delivery one or more drugs to one or more of site-specific ocular location from a formulation identical to or different from one another of drug formulation (drug type, volume, amount, and ingredient etc., or a combination of these parameters). For example, two or more microneedles can be inserted into the ocular tissue, each microneedle may form an independent pocket in the ocular tissue into or through which a drug formulation may be deposited.

Further, the first microneedles can be inserted into the ocular tissue with formulation to generate a drug to ocular tissue with a burst release rate and the second microneedles can be inserted into the ocular tissue with formulation to generate a drug to ocular tissue with a sustained or extended-release rate. Each microneedle may form an independent pocket in the ocular tissue into or through which a drug formulation may be deposited. Alternatively, a first microneedle may be used to deliver the anti-VEGF agent and a second microneedle may be used to deliver the direct factor Xa inhibitor and the direct factor IIa inhibitor.

The microneedle can be formed an implant from a drug encapsulated within a biodegradable/bioerodable polymer, which can be implanted into the ocular tissue for drug release upon degradation/dissolution of the microneedle. The microneedle can also be formed of drug coated metal and delivers the drug by dissolution/diffusion upon insertion into the ocular tissue.

In some examples, a drug delivery system comprised a drug coated contact lens. The drug can be nano dispersed and diffuse into or migrate through contact lens and into the post-lens tear film when contact lens is placed on the eye.

In some examples, a drug delivery system comprised Port Delivery System (PDS). The PDS is designed to be a permanent, reusable drug reservoir as a refillable eye implant as the size of a grain of rice to continuously deliver a drug over a period of months, potentially reducing the treatment burden associated with frequent eye injections. It is initially placed as an implant, and subsequent refills with a specialized needle that flushes out the contents of the device while at the same time refilling it with fresh drug in a doctor office. At the distal end of the septum of the needle, there is a semipermeable titanium membrane that permits continuous passive diffusion of the drug from the higher concentration in the reservoir into the vitreous. The reservoir capacity is from about 0.005 cc to about 0.2 cc. The length of the device is no more than about 6 mm as the size of a grain of rice from the sclera into the vitreous has a minimal effect on patient vision. Extended or sustained release is achieved with the therapeutic agent released from the therapeutic device PDS after implantation. Upon release at the vitreous, the therapeutic agent is stable in the device for at least 30 days. In some cases, the therapeutic agent is released from the device for at least 6 months.

Controlled delivery implants may be formed from biodegradable/erodible polymer or water-soluble polymers matrix. The suitable polymer include but not limited to polyesters, polylactide, polyglycolide, poly(ε-caprolactone), polydioxanone, poly(hydroxyalkanoates), poly(L-lactide-co-D-lactide), poly(L-lactide-co-D,L-lactide), poly(D-lactide-co-D,L-lactide), poly(lactide-co-glycolide) (including 70:30 to 99:1 PLA-co-PGA, such as 85:15 PLA-co-PGA), poly(lactide-co-ε-caprolactone) (including 70:30 to 99:1 PLA-co-PCL, such as 90:10 PLA-co-PCL), poly(glycolide-co-ε-caprolactone), poly(lactide-co-dioxanone), poly(glycolide-co-dioxanone), poly(lactide-co-trimethylene carbonate), poly(glycolide-co-trimethylene carbonate), poly(lactide-co-ethylene carbonate), and copolymers and combinations thereof, wherein lactide includes L-lactide, D-lactide and D,L-lactide, block copolymer of ethylene oxide and propylene oxide, di-block copolymer of polyethylene oxide and polypropylene oxide, tri-block copolymer of polyethylene oxide and polypropylene oxide, and copolymers and combinations thereof.

### Experimental Examples

Examples 1 through 29 describe coating implants in the form of stents, such as vascular stents and drug delivery balloons. The methods for implant fabrication and the successful elution of drugs described under vascular conditions for these implants would apply as well to the fabrication of ocular implants, such as shunts, drug coated microneedles, and ocular stents.

### Example 1: Preparation of Anticoagulant (Rivaroxaban, Argatroban, and Dalteparin) Eluting Implants

Poly(n-butyl methacrylate) polymer was dissolved into dichloromethane (tetrahydrofuran (THF) was used for Dalteparin) at room temperature and vortexed until the polymer had uniformly dissolved/dispersed. Rivaroxaban was dissolved into dichloromethane at room temperature and vortexed until the drug was uniformly dissolved/dispersed. Argatroban (and Argatroban in combination with Rivaroxaban) was dissolved in Methanol and dichloromethane and vortexed at room temperature until the drug was uniformly dispersed/dissolved. Dalteparin was dissolved in water and THF until fully dissolved.

Each polymer solution and each drug solution were combined together (Rivaroxaban to poly(n-butyl methacrylate) by weight ratio was 6:1), (Argatroban to poly(n-butyl methacrylate) by weight ratio was 3:4), (Dalteparin to poly(n-butyl methacrylate) by weight ratio was 2:3), and (Rivaroxaban in combination with Argatroban to Poly (n-butyl methacrylate) weight ratio was 3:2:2) according to the target drug dose of 150 µg for each drug (and 100 µg each for the Rivaroxaban and Argatroban combination).

A microprocessor-controlled ultrasonic sprayer was used to coat each of the implant (a stent but these methods would work as well for ocular shunts and other ocular implants) over 14 mm length uniformly with each of the drug/polymer matrix solutions. After coating, the implants were placed in a vacuum chamber to remove the solvent. The stent implants were then mounted on balloon catheters and crimped. The catheters were then inserted in coils and packaged. The pouches were sterilized. The bare metal control stents were the same as the other stents without a drug or polymer coating.

### Example 2: In Vivo Testing of Drug Eluting Stent With Different Drugs

The drug eluting stent systems containing different anticoagulants prepared as described in Example 1 were evaluated at 3 hours, 6 hours, 1 day, 3 days, 6 days, 7 days, or 28 days following implantation in a porcine coronary artery model.

The porcine model was chosen as this model has been used extensively for stent and angioplasty studies resulting in a large volume of data on the pulmonary response properties and its correlation to human pulmonary response (Schwartz et al, Circulation. 2002; 106:1867 1873). The animals were housed and cared for in accordance with the Guide for the Care and Use of Laboratory Animals as established by the National Research Council.

After induction of anesthesia, the left or right femoral artery was accessed using standard techniques and an arterial sheath was introduced and advanced into the artery. Vessel angiography was performed under fluoroscopic guidance, a 7 Fr. guide catheter was inserted through the sheath and advanced to the appropriate location where intracoronary nitroglycerin was administered. An appropriate implantation segment of coronary artery was randomly selected and a 0.014” guidewire inserted. Quantitative Coronary Angiography (QCA) was performed to document the results. The appropriately-sized stent was advanced to the deployment site. The balloon was inflated at a steady rate to a pressure sufficient to achieve a balloon to artery ratio of approximately 1.1 to 1.0 but less than 1.2:1.

Follow up angiography was performed at the designated timepoint for each of the animals. Late lumen loss (LLL) can be expressed as: LLL = Post-stent minimum lumen diameter - Final minimum lumen diameter The LLL is an indicator of the amount smooth muscle cell (SMC) proliferation or inhibition. It is used to measure efficacy between drugs for SMC proliferation inhibition. The smaller the LLL, the better the efficacy of the drug.

Stented portions of coronary arteries were embedded in methyl methacrylate (MMA), then divided into a target of at least three blocks of approximately similar lengths for histology evaluation. Quantitative histopathological evaluation of stented artery sections was then performed and scored as indicated. For Fibrin formation, scores ranged from 0 to 3, with a score of 0 indicating absent or rare minimal spotting around struts of the stent, a score of 1 indicating the presence of fibrin in small amounts localized only around the struts, a score of 2 indicating the moderately abundant or denser presence of fibrin around and extending beyond the struts, and a score of 3 indicating the presence of abundant and dense fibrin and/or bridging of the fibrin between the sruts. The mean score was calculated and reported. The mean of each section was then averaged to provide a mean fibrin score per stent. The smaller the fibrin score, the better efficacy.

The percentage and/or amount of each anticoagulant drug for or by each time point indicated were analyzed for each stent from the different devices in the example and the average drug tissue concentration reported.

Tissue concentrations and the amount of drug released from the stents were measured using stents implanted in porcine arteries for the drugs as indicated. The arteries at the designated time point were excised and a length of stented artery spanning from 5 mm proximal to the stented segment to 5 mm distal to the stented segment was cut. The stented artery was cut longitudinally with surgical scissors. The stents were separated from the tissue. The tissue content of each drug was analyzed using liquid chromatography mass spectroscopy (LCMS) and reported as a mean for each of the timepoint indicated. For drug remaining on each stent, each drug was extracted from the stent, measured using HPLC, and reported as a mean for each of the timepoint indicated as drug released or drug remaining on a stent (where drug remaining is equal to 100% minus the percentage of drug released).

TABLE 1

Histopathology Scores, Quantitative Coronary Angiography data and PK data of Rivaroxaban, Argatroban, and Dalteparin (low molecular weight heparin) released from 14 mm stents at day 7

| Stent coating information (n=3 for each arm) | Fibrin score at day 7 | Cumulative Percent Release of drug by day 7 | Tissue concentration at day 7 (ng/mg) | LLL at day 7 | Injury | Inflammation | Diameter Stenosis from Coronary (%) |
|---|---|---|---|---|---|---|---|
| 150 µg Rivaroxaban & 25 µg Poly(n-butyl methacrylate) matrix coated stent | 0.72±0.12 | 99.6% | 3.9±0.6 | 0.31±0.20 | 0.03±0.04 | 1.15±0.34 | 11.4±2.5 |
| 150 µg Argatroban & 200 µg Poly(n-butyl methacrylate) matrix coated stent | 1.25±0.87 | 47.7% | 5.7±1.8 | 0.34±0.20 | 0.08 ± 0.08 | 0.81 ± 0.46 | 2.4±2.3 |
| 100 µg Argatroban and 100 µg Rivaroxaban in 150 µg Poly(n-butyl methacrylate) matrix coated stent | 0.80±0.39 | 70.9% for Rivaroxaban 96.5% for Argatroban | 48.1±43.6 for Rivaroxaban 8.9±6.3 for Argatroban | 0.04±0.06 | 0.06 ± 0.07 | 1.11 ± 0.35 | 0.5±0.9 |
| 150 µg Dalteparin(low molecular weight heparin) in 225 µg Poly(n-butyl methacrylate) matrix coated stent | 1.50±0.82 | 98.3% | Not tested | 0.23±0.20 | 0.05±0.06 | 0.65±0.30 | 3.7±5.2 |
| Bare metal control stent (BMS) | 1.02±0.35 | N/A | N/A | 0.17±0.22 | 0.05 ± 0.08 | 0.77 ± 0.45 | 3.3±1.7 |

As shown in Table 1, Rivaroxaban composition released from stents was more effective at inhibiting fibrin formation compared to bare metal control stents at 7 days, while Argatroban composition released from stents or Dalteparin composition released from stents were not more effective at inhibiting fibrin formation compared to bare metal control stents at 7 days.

As shown in Table 1, Rivaroxaban, Argatroban, or Dalteparin compositions released from stents as single agents had larger LLLs compared to control and thus were not more effective at inhibiting smooth muscle cell proliferation compared to bare metal control stents at 7 days.

As shown in Table 1, the combination of Rivaroxaban and Argatroban composition released from stents had a smaller LLL compared to control and thus was more effective at inhibiting smooth muscle cell proliferation compared to bare metal control stents at 7 days. Furthermore, the combination of Rivaroxaban and Argatroban composition released from stents was more effective at inhibiting fibrin formation compared to bare metal control stents.

As shown in Table 1, Rivaroxaban composition comprising fast released from stents at a dose of about 150 µg within 7 days from implant (or from vessel injury) was more effective at inhibiting fibrin formation at or within 7 days.

As shown in Table 1, Rivaroxaban composition comprising fast released from stents at a dose of about 1.8 µg/mm2 within 7 days from implant (or from vessel injury) was more effective at inhibiting fibrin formation at or within 7 days.

As shown in Table 1, Rivaroxaban composition comprising fast released from stents at a dose of about 10.7 µg/mm of stent length within 7 days from implant (or from vessel injury) was more effective at inhibiting fibrin formation.

As shown in Table 1, Rivaroxaban composition comprising a dose of about150µg, and/or of about 1.8 µg/mm², and/or of about 10.7 µg/mm of device length, released from a stents device at a release rate comprising of about 99.5% within 7 days from implant (or from time of injury) was more effective at inhibiting fibrin formation.

As shown in Table 1, Rivaroxaban composition released from stents at a release rate comprising of about 70.9% within 7 days when combined with Argatroban composition at a release rate comprising of about 96.9% within 7 days from implant (or from time of injury) was more effective at inhibiting fibrin formation.

Table 1 shows Rivaroxaban composition released from a stent at a dose of about 100 µg, or at a dose comprising of about 1.2 µg/mm², and/or at a dose of about 7.14 µg/mm of stent length, at a release rate comprising of about 70.9% within 7 days when combined with Argatroban composition released from a stent at a dose comprising of about 100 µg, and/or at a dose of about 1.2 µg/mm², and/or at a dose of about 7.14 µg/mm of stent length, at a release rate comprising of about 96.5% within 7 days from implant (or from time of injury) was more effective at inhibiting fibrin formation.

### Example 3: Preparation of Rivaroxaban and m-TOR Inhibitor Releasing Implant

Base coat of Novolimus (m-TOR inhibitor) and Poly (n-butyl methacrylate) matrix: Poly(n-butyl methacrylate) polymer was dissolved into dichloromethane at room temperature and vortex until the polymer had uniformly dissolved/dispersed. Novolimus was placed in another vial and dissolved in dichloromethane at room temperature until uniformly dissolved or dispersed. The polymer solution and drug solutions were mixed together and coated as a matrix (the drug to polymer weight ratio was 2:3 by weight).

Top layer or coat of Rivaroxaban and poly (n-butyl methacrylate) matrix: Poly(n-butyl methacrylate) polymer was dissolved in dichloromethane at room temperature and vortex until the polymer had uniformly dissolved/dispersed. Rivaroxaban was dissolved into dichloromethane at room temperature and vortex until the drug was uniformly dissolved/dispersed. Each polymer solution and each drug solutions were mixed together as a matrix (Rivaroxaban to poly(n-butyl methacrylate) by weight ratio was 6:1 for the Rivaroxaban fast formulation without m-TOR). Rivaroxaban to poly (n-butyl methacrylate) ratio was 4:1 for the fast release formulation with m-TOR base coat matrix, and 2:1 for the slow release formulation with m-TOR base coat matrix according to the target drug dose of 100 µg Rivaroxaban and 25 µg poly(n-butyl methacrylate) for fast release formulation, and 100 µg Rivaroxaban and 50 µg poly(n-butyl methacrylate) for the slow release formulation.

A microprocessor-controlled ultrasonic sprayer was used to coat each of the implants (the stents but these methods would work as well for ocular shunts and other ocular implants) ’ 14 mm length uniformly with each of the drug/polymer matrix solution with the base coat matrix first, placing the stents in vacuum chamber to remove the solvent, followed by the top layer or coat matrix. The stents were placed in a vacuum chamber again to remove the solvents. The stents were then mounted on balloon catheters and crimped. The catheters were then inserted in coils and packaged. The pouches were sterilized. The Novolimus (m-TOR inhibitor) stents controls (DES) consisted of only the base coat drug/polymer matrix, without the top layer or coat drug/polymer matrix, otherwise being the same as the other stents. The bare metal control stents (BMS) were the same as the other stents without a drug or polymer coating.

[symbol]

As shown in Table 2, Rivaroxaban composition comprising fast released formulation from a stent was more effective at inhibiting fibrin formation compared to control at 7 days and/or at 28 days when it was released at a faster rate formulation.

As shown in Table 2, Rivaroxaban composition released from a stent was more effective at inhibiting fibrin formation compared to control at 7 days and/or at 28 days when it was released at a rate comprising of about 88.9% within 7 days and/or when a dose of about 88.9 µg, and/or a dose of about 1.2 pg/mm², and/or a dose of about 7.14 µg/mm of stent length, was released within 7 days from vessel injury (or from implantation).

As shown in Table 2, Rivaroxaban formulation released from a stent was more effective at inhibiting fibrin formation compared to control at 7 days and/or at 28 days when it was released at a rate comprising of about 92.5% within 28 days and/or when a dose of about 92.5 µg, and/or a dose of about \1.1 pg/mm², or a dose of about 6.6 µg/mm of stent length, was released at or within 28 days from vessel injury (or from implantation).

As shown in Table 2, Rivaroxaban composition released from a stent was more effective at inhibiting fibrin formation compared to control at 7 days and/or at 28 days when it was released at a rate comprising of 88.9% within 7 days and/or when a dose of 88.9 µg, and/or a dose of 1.2 µg/mm², and/or a dose of 7.14 µg/mm of stent length, was released within 7 days after vessel injury (or from implantation), and/or at a rate comprising of about 92.5% within 28 days and/or when a dose of about 92.5 µg, and/or a dose of about 1.1 pg/mm², and/or a dose of about 6.6 µg/mm of stent length, was released within 28 days after vessel injury (or from implantation).

As shown in Table 2, Rivaroxaban composition released in combination with an m-TOR inhibitor from a stent was more effective at inhibiting fibrin formation compared to control at 7 days and/or at 28 days when it was released in a faster formulation rate in accordance with the experiment.

As shown in Table 2, Rivaroxaban composition released in combination with an m-TOR formulation inhibitor from a stent was more effective at inhibiting fibrin formation compared to control at 7 days and/or at 28 days when Rivaroxaban composition was released at a rate comprising of about 88.9 µg, or a dose of about 1.2 pg/mm², or a dose of about 7.14 µg/mm of stent length, within 7 days, and/or released at a rate comprising of about 92.5 µg, and/or a dose of about 1.1 pg/mm², and/or a dose of about 6.6 µg/mm of stent length, within 28 days.

As shown in Table 2, Rivaroxaban composition released in combination with an m-TOR inhibitor formulation from a stent was more effective at inhibiting fibrin formation compared to control at 7 days when Rivaroxaban composition was released at a rate comprising of about 68.1 µg, or at rate comprising of 0.84 pg/mm², and/or at a rate comprising of about 4.86 µg/mm of stent length, within 7 days.

As shown in Table 2, Rivaroxaban tissue concentration ranges from at least 3.96 ng/mg of tissue adjacent to the stented segment to at least 15 ng/mg of tissue adjacent to the stented segment, within or at 7 days, or within or 28 days from implant (or tissue injury)

It was reported that Rivaroxaban IC₅₀ for factor Xa inhibition to be about 21 nM or 0.0092 ng/mg. As shown in Table 2, the tissue concentration for Rivaroxaban was at least 426 times Rivaroxaban IC 50 for factor Xa inhibition.

TABLE 3

Tissue concentration of Rivaroxaban and Argatroban at 7 days show multiple folds higher (or times higher) than IC50 for Anti-factor Xa/IIa and antiplatelet for the respective drugs

| Stent coated with combination of Rivaroxaban and Argatroban | Rivaroxaban* | | Argatroban* * | |
|---|---|---|---|---|
| | Tissue concentration at day 7 in folds higher than IC50 of anti-Factor Xa | Tissue concentration at day 7 in folds higher than IC50 of anti-platelet | Tissue concentration at day 7 in folds higher than IC50 of anti-Factor IIa | Tissue concentration at day 7 in folds higher than IC50 of anti-platelet |
| 100 µg Argatroban and 100 µg Rivaroxaban in Poly(n-butyl methacrylate) matrix | 5253 | 354 | 835 | 1223 |

* Rivaroxaban IC50 for Anti-Factor Xa is 21 nM or 0.00916 ng/mg
*Rivaroxaban IC50 for Tissue factor generated antiplatelet is 312 nM or 0.136 ng/mg
**Argatroban IC50 for Anti-Factor IIa is 21 nM or 0.0107 ng/mg
**Argatroban IC50 for Tissue factor generated antiplatelet is 79 nM or 0.04 ng/mg

Table 3 shows the tissue PK data for Rivaroxaban and Argatroban at or by or within 7 days from implants of stented vessels. It shows Rivaroxaban and Argatroban has therapeutic tissue concentrations in the tissue segment up to 7 days. Table 3 is Rivaroxaban and Argatroban concentration (ng/mg) in the tissue of treated area of the implanted device fold higher than IC₅₀ for anti-Factor Xa or Anti-Factor IIa and anti-platelet. It shows that Rivaroxaban and Argatroban in tissue concentrations have several order of magnitudes, has from 2 to 4 orders of magnitude of tissue concentration for each of the drugs compared to their IC₅₀, in the treated tissue segments up to 7 days, therefore inhibiting or enhancing dissolution of one or more of cell proliferation, fibrin formation, or clot formation on the device surfaces, the stented segment tissue, and/or the tissue adjacent to the stented segment.

### Example 4: Preparation of Anticoagulant1/Anticoagulant2/mTOR Eluting Implants

Poly(L-lactide acid-co-glycolic acid) polymer was dissolved into dichloromethane at room temperature and vortex until the polymer had uniformly dissolved/dispersed. Sirolimus and anticoagulants (Apixahan or Rivaroxaban and Argatroban) were placed in a vial and dissolved in dichloromethane or dichloromethane/Methanol at room temperature and vortex until all the drug was uniformly dissolved/dispersed.

Each polymer solution and each drug (or combined drugs) solutions were combined together (SS7 arm anticoagulant (Apixaban to Argatroban was 1:1) to poly(L- lactide acid-co-glycolic acid) matrix by weight ratio was 3:1 as a base coat and Siroliums to poly(L-lactide acid-co-glycolic acid) matrix by weight ratio was 2:3 and coated as a top coat), (SS9 arm Siroliums and anticoagulant Apixaban and Argatroban was (1:1:1) to poly(L- lactide acid-co-glycolic acid) by weight ratio was 5:2 on matrix), (SS15 arm Sirolimus and Apixaban and Argatroban were combined in a ratio of (1:1:1) with poly(L- lactide acid-co- glycolic acid) by weight ratio which was (1:2)( by weight of 23 µg Sirolimus, 23 µg Apixaban and 23 µg Argatroban combined with 138 µg poly(L- lactide acid-co- glycolic acid)) and mixed together, and coated as a base coat (drug/polymer matrix as a base coat). In addition, Sirolimus and Apixaban and Argatroban were combined in the ratio of (3:4:4) with poly(L- lactide acid-co-glycolic acid) by weight ratio which was (5:3) and coated as a top layer or coat (drug/polymer matrix top layer or coat), (by weight of 71 µg Sirolimus, 94 µg Apixaban and 94 µg Argatroban and combined with 155 µg poly(L- lactide acid-co- glycolic acid) and coated as a top layer or coat, for cumulative total target drug dose of 117 µg for each anticoagulant and 94 µg for Sirolimus for a 14 mm stent length, (Slider II Arm1 (SS16) Sirolimus and Rivaroxaban and Argatroban were combined together in the ratio of (1:1:1) and were combined with poly(L-lactide acid-co- glycolic acid) by weight ratio which was (1:2) and coated as a base coat (drug/polymer matrix as base coat). In addition, Sirolimus and Rivaroxaban and Argatroban were combined in the ratio of (3:4:4) and combined with poly(L- lactide acid-co- glycolic acid) by weight ratio was (5:3) and coated as a top layer or coat (drug/polymer matrix as top layer or coat), (by weight of 23 µg Sirolimus, 23 µg Rivaroxaban and 23 µg Argatroban and 138 µg poly(L- lactide acid-co- glycolic acid) mixed together and coated as base coat; and by weight of 71 µg Sirolimus, 94 µg Rivaroxaban and 94 µg Argatroban and 155 µg poly(L- lactide acid-co-glycolic acid) mix together in a matrix and coated as top layer or coat, for a total target drug dose of 117 µg for each anticoagulant and 94 µg for Sirolimus for a 14 mm stent length, (Slider II Arm2 (SS17) Sirolimus and Rivaroxaban and Argatroban were combined in a ratio of (4:1:1) and combined with poly(L- lactide acid-co- glycolic acid) by weight ratio which was (1:1) and coated as a base coat (drug/polymer matrix as base coat). In addition, Rivaroxaban and Argatroban were combined in a ratio of (1:1) and combined with poly(L- lactide acid-co-glycolic acid) by weight ratio which was (5:3) and coated as a top layer or coat on the stent (drug/polymer matrix as a top layer or coat), (by weight of 94 µg Sirolimus, 23 µg Rivaroxaban and 23 µg Argatroban and 140 µg poly(L- lactide acid-co- glycolic acid) mixed together and coated as base coat; and by weight of 94 µg Rivaroxaban and 94 µg Argatroban and 113 µg poly(L- lactide acid-co- glycolic acid) were mixed together and coated as top layer or coat, for a total target drug dose of 117 µg for each anticoagulant and 94 µg for Sirolimus for a 14 mm stent length. The preceding doses for SS7, SS9, SS15, SS16, and SS17 were for 14 mm stent lengths. Drug and polymer doses are adjusted accordingly for each stent length. Control was 14 mm stent length eluting 65 µg Novolimus (m-TOR inhibitor). A microprocessor controlled ultrasonic sprayer was used to coat each of the implants’ (the stents but these methods would work as well for ocular shunts and other ocular implants) 14 mm length uniformly with each of the drug/polymer matrix solution. After coating, the stents were placed in a 70° C. oven for about 2 hours to remove the solvent. The stents were then mounted on balloon catheters and crimped. The catheters were then inserted in coils and packaged. The pouches were sterilized.

The following tables 4A-4K describe results from in vivo testing for the following arms of SS7, SS9, SS15,SS16, and SS17 from example 4.

TABLE 4A

In-vivo cumulative percent drug release profile of Rapamycin, Apixaban/ Rivaroxaban and Argatroban in stented segments

| Sample Matrix | Sample Size | Time period | 1H | 3H | 24H | 6D | 7D | 28D | 90D |
|---|---|---|---|---|---|---|---|---|---|
| SS7 Argatroban/Apixaban /Sirolimus | n=1 | Apixaban,% | N/A | 30 | 91 | 98 | 99 | 99 | N/A |
| | | Argatroban,% | N/A | 91 | 99 | 98 | 99 | 99 | N/A |
| | | Sirolimus,% | N/A | 50 | 70 | 69 | 73 | 77 | N/A |
| SS9 Argatroban/Apixaban /Sirolimus | n=1 | Apixaban,% | N/A | 68 | 97 | 98 | 98 | 99 | N/A |
| | | Argatroban,% | N/A | 79 | 98 | 98 | 98 | 99 | N/A |
| | | Sirolimus,% | N/A | 60 | 91 | 93 | 94 | 97 | N/A |
| SS15 Argatroban/Apixaban /Sirolimus | n=5 | Apixaban,% | 49 | 61 | 77 | N/A | 80 | 84(n=3) | 87(n=3) |
| | | Argatroban,% | 51 | 63 | 77 | N/A | 80 | 84(n=3) | 86(n=3) |
| | | Sirolimus,% | 44 | 55 | 71 | N/A | 81 | 90(n=3) | 97(n=3) |
| SS16 Argatroban/Rivaroxaban /Sirolimus | n=5 | Rivaroxaban,% | 36 | 47 | 83 | N/A | 86 | 89 | N/A |
| | | Argatroban,% | 35 | 49 | 82 | N/A | 85 | 87 | N/A |
| | | Sirolimus,% | 29 | 44 | 73 | N/A | 87 | 94 | N/A |
| SS17 Argatroban/Rivaroxaban /Sirolimus | n=5 | Rivaroxaban,% | 65 | 71 | 86 | N/A | 92 | 94 | N/A |
| | | Argatroban,% | 78 | 80 | 86 | N/A | 91 | 93 | N/A |
| | | Sirolimus,% | 8 | 18 | 63 | N/A | 77 | 87 | N/A |

N/A: Not available

Table 4A: SS7 and SS9 provide a therapeutic composition where about 90% of the factor Xa and factor IIa inhibitors are released from the stent within 24 hours. It also shows that these agents are released substantially completely within about 28 days.

Table 4A: SS15, SS16, and SS17 provide therapeutic compositions where each composition providing a bolus drug release from time of injury and/or implant, and an extended drug release from time of injury and/or implant for each of Apixaban, Rivaroxaban, and Argatroban.

Table 4A: SS15 provides a therapeutic composition providing a bolus drug release phase (or formulation) from time of injury and/or implant and an extended drug release phase (or formulation) from time of injury and/or implant for the combination of Apixaban and Argatroban, wherein the bolus drug release occurs within an hour, within 3 hours, or within 24 hours, from time of injury and/or implant; and the extended drug release extends beyond 7 day, extends beyond 28 days, or extends beyond 90 days from time of injury and/or implant.

Table 4A: SS15 provides a therapeutic composition a bolus drug release phase (or formulation) from time of injury and/or implant and an extended drug release phase (or formulation) from time of injury and/or implant for the combination of Apixaban, Argatroban, and Sirolimus, wherein the bolus drug release occurs within an hour to within 24 hours from time of injury and/or implant and the extended drug release extends beyond 7 day, extends beyond 28 days, or extends beyond 90 days from time of injury and/or implant.

Table 4A: SS15 provides a therapeutic composition providing a bolus drug release phase (or formulation) from time of injury and/or implant and an extended drug release phase (or formulation) from time of injury and/or implant for the combination of Apixaban and Argatroban, wherein the bolus drug release occurs within an hour from time of injury and/or implant and wherein Apixaban bolus release is about 49% within an hour and wherein Argatroban bolus release is about 51% within an hour and the extended drug release of each of the drugs is about 80% within 7 days, about 84% within 28 days, and about 86% within 90 days from time of injury and/or implant. In this arm, the drugs are released or commence release substantially about the same time.

Table 4A: SS16 and SS17 provide therapeutic composition providing a bolus drug release phase (or formulation) from time of injury and/or implant and an extended drug release phase (or formulation) from time of injury and/or implant for the combination of Rivaroxaban and Argatroban, wherein the bolus drug release occurs within an hour to within 24 hours from time of injury and/or implant and the extended drug release extends beyond 7 day, or extends beyond 28 days from time of injury and/or implant.

Table 4A: SS16 provides a therapeutic composition providing a bolus drug release phase (or formulation) from time of injury and/or implant and an extended drug release phase (formulation) from time of injury and/or implant for the combination of Rivaroxaban, Argatroban, and Sirolimus, wherein the bolus drug release occurs within an hour to within 24 hours from time of injury and/or implant and the extended drug release extends beyond 7 day, or extends beyond 28 days from time of injury and/or implant. In this arm, the drugs are released or commence release substantially about the same time.

Table 4A: SS16 and SS17 provide therapeutic compositions providing a bolus drug release phase (or formulation) from time of injury and/or implant and an extended drug release phase (or formulation) from time of injury and/or implant for the combination of Rivaroxaban and Argatroban, wherein the bolus drug release occurs within an hour to within 24 hours from time of injury and/or implant and wherein Rivaroxaban bolus release ranges from 36% to 68% within an hour and wherein Argatroban bolus release ranges from 35% to 78% within an hour and the extended drug release of each of the drugs ranges from 85% to 92% for Rivaroxaban within 7 days, 86%-91% for Argatroban within 7 days, ranges from 89%-94% within 28 days for Rivaroxaban and 87%-93% for Argatroban from time of injury and/or implant to within 28 days.

Table 4A: SS16 and SS17 formulations each has one formulation providing a bolus drug release and another formulation providing an extended drug release for the combination of Rivaroxaban and Argatroban, wherein the bolus drug release occurs within an hour to within 24 hours of injury or implantation and the extended drug release extends beyond 7 day, or extends beyond 28 days.

Table 4A: SS16 and SS17 shows multiple formulations providing a bolus drug release formulation and an extended drug release formulation for the combination of each of Rivaroxaban and Argatroban, wherein the extended release extends beyond 7 day, or extends beyond 28 days.

Table 4A: SS15, SS16, and SS17 Provides therapeutic compositions comprising two drugs/polymer formulations each, wherein each formulation contains at least two drugs: a factor Xa inhibitor and a factor IIA inhibitor. A third drug being an M-tor inhibitor is present in each of the formulations except in SS17 where it is present in only one formulation (base formulation) configured to delay the release of M-tor in SS17 providing a smaller bolus within the first hour for M-tor. All formulations provide an extended release of the drugs beyond 7 days, or beyond 28 days. Arm SS17 factor IIa inhibitor and factor Xa inhibitor commence release prior to the anti-proliferative which was intended/configured to delay commence of its release compared to the other two drugs.

TABLE 4B

Tissue drug concentration (ng/mg) of Apixaban, Rivaroxaban, Argatroban and Rapamycin in the stented segment tissue at the indicated time points following implantation

| Sample Matrix | Sample Size | Time period | 1H | 3H | 24H | 6D | 7D | 28D | 90D |
|---|---|---|---|---|---|---|---|---|---|
| SS7 Argatroban /Apixaban /Sirolimus | n-1 | Apixaban | N/A | 102.9 | 16.5 | 0.07 | 0.03 | 0.12 | N/A |
| | | Argatroban | N/A | 54 | 0.75 | 0.09 | 0.05 | 0.15 | N/A |
| | | Sirolimus | N/A | 7.34 | 4.46 | 1.38 | 0.73 | 1.79 | N/A |
| SS9 Argatroban /Apixaban /Sirolimus | n-1 | Apixaban | N/A | 91.8 | 4.17 | 0.28 | 2.65 | 1.69 | N/A |
| | | Argatroban | N/A | 123.1 | 0.18 | 0.33 | 2.54 | 1.76 | N/A |
| | | Sirolimus | N/A | 40.57 | 0.93 | 1.61 | 3.24 | 2.48 | N/A |
| SS15 Argatroban /Apixaban /Sirolimus | n=5 | Apixaban | 66.94 ± 27.33 | 25.31 ± 11.21 | 13.25 ± 10.17 | NA | 1.15 ± 0.52 | 1.28 ± 0.47(n=3) | 3.05 ± 1.77(n=3) |
| | | Argatroban | 71.37 ± 31.32 | 27.65 ± 15.00 | 15.64 ± 12.08 | NA | 1.41 ± 0.69 | 1.69 ± 0.64(n=3) | 3.74 ± 1.89(n=3) |
| | | Sirolimus | 43.22 ± 14.73 | 23.37 ± 6.88 | 29.17 ± 18.65 | NA | 1.54 ± 0.35 | 1.67 ± 0.22(n=3) | 1.28 ± 0.07(n=3) |
| SS16 Argatroban /Rivaroxab an /Sirolimus | n=5 | Rivaroxaban | 48.75 ± 25.52 | 21.480, 5.80 | 3.67± 5.59 | N/A | 0.31 ± 0.24 | 0.34 ±0.27 | N/A |
| | | Argatroban | 61.87 ± 24.60 | 32.81± 10.96 | 3.80± 4.87 | N/A | 0.42 ± 0.32 | 0.52 ±0.37 | N/A |
| | | Sirolimus | 45.10 ± 14.77 | 38.30 ± 9.29 | 9.18 ±5.69 | N/A | 1.46 ± 0.38 | 0.94 ± 0.19 | N/A |
| SS17 Argatroban /Rivaroxab an /Sirolimus | n=5 | Rivaroxaban | 38.31± 16.08 | 26.23 ± 23.50 | 1.31 ± 0.28 | N/A | 1.07 ± 1.88 | 0.52 ± 0.63 | N/A |
| | | Argatroban | 11.80 ± 2.69 | 8.06± 3.48 | 1.19 ± 0.46 | N/A | 1.35 ± 2.41 | 0.67 ± 0.87 | N/A |
| | | Sirolimus | 21.34 ± 7.51 | 27.32 ± 6.86 | 10.85 ± 3.55 | N/A | 3.80 ± 4.86 | 1.73 ± 1.52 | N/A |

Table 4B shows drug concentration in tissue adjacent to the stented segment for each of the drugs: Apixaban of about 67 ng/mg within one hour, of about 25 ng/mg tissue within 3 hours, of about 1.15 ng/mg tissue within 7 days, 1.28 ng/mg tissue within 28 days, and of about 3 ng/mg tissue within 90 days from time of injury and/or implant; Rivaroxaban of about 38 ng/mg, or of about 49 ng/mg within one hour, of about 21 ng/mg, or of about 26 ng/mg tissue within 3 hours, of about 0.3 ng/mg, or of about 1.1 ng/mg tissue within 7 days, of about 0.34 ng/mg, or of about 0.52 ng/mg tissue within 28 days, from time of injury and/or implant; Argatroban of about 12 ng/mg, of about 62 ng/mg tissue, or of about 71 ng/mg tissue within 1 hour, of about 8 ng/mg tissue, of about 33 ng/mg tissue, or of about 27 ng/mg tissue within 3 hours, of about 0.42 ng/mg tissue, of about 1.35 ng/mg tissue, or of about 1.41 ng/mg tissue within 7 days, of about 0.52 ng/mg tissue, of about 0.67 ng/mg tissue, or of about 1.69 ng/mg tissue within 28 days, and of about 3.74 ng/mg tissue within 90 days from time of injury and/or implant; and Sirolimus of about 21 ng/mg tissue, of about 45 ng/mg tissue, or of about 43 ng/mg tissue within one hour, of about 27 ng/mg tissue, or about 38 ng/mg tissue, or of about 24 ng/mg tissue within 3 hours, of about 1.46 ng/mg tissue, of about 3.8 ng/mg tissue, or of about 1.54 ng/mg tissue within 7 days, of about 0.94 ng/mg tissue, of about 1.73 ng/mg tissue, or of about 1.67 ng/mg tissue within 28 days, and of about 1.28 ng/mg tissue within 90 days, from time of tissue injury and/or implant.

TABLE 4C

In Vivo drug remaining on stent (µ g) and average cumulative percentage releases (%) of Apixaban, Rivaroxaban, Argatroban and Sirolimus in the stent at the indicated time points following implantation

| Sample matrix* (n=5) | SS15 Argatroban/Apixaban /Sirolimus in base coat and in topcoat | | | SS16 Argatroban/Rivaroxaban /Sirolimus in base coat and in topcoat | | | SS17 Argatroban/Rivaroxaban /Sirolimus in base coat and in topcoat | | |
|---|---|---|---|---|---|---|---|---|---|
| Drug remaining on stent, µg & percentage released (%) | Apixaban | Argatroban | Sirolimus | Rivaroxaban | Argatroban | Sirolimus | Rivaroxaban | Argatroban | Sirolimus |
| Time (hrs) | | | | | | | | | |
| 0 | 119 | 123 | 96 | 120 | 119 | 90 | 121 | 121 | 96 |
| 1H | 61±4.8 (49%) | 60±5.5 (51%) | 54±2.8 (44%) | 76±7.1 (36%) | 77±59.2 (35%) | 63±4.7 (29%) | 43±3.5 (65%) | 26±0.8 (78%) | 88±1.5 (8%) |
| 3H | 46±5.2 (61%) | 46±5.1 (63%) | 43±3.9 (55%) | 64±9.5 (47%) | 61±13.9 (49%) | 50±6.1 (44%) | 34±5.1 (71%) | 24±1.9 (80%) | 79±3.5 (18%) |
| 24H | 27±3.6 (77%) | 28±1.4 (77%) | 28±0.8 (71%) | 21±4.8 (83%) | 21±1.6 (82%) | 24±7.1 (73%) | 17±5.9 (86%) | 17±3.8 (86%) | 36±7.3 (63%) |
| 7D | 24±1.4 (80%) | 25±1.0 (80%) | 18±0.3 (81%) | 16±2.5 (86%) | 18±1.7 (85%) | 12±1.2 (87%) | 9±0.3 (92%) | 11±0.4 (91%) | 22±0.5 (77%) |
| 28D | 19±0.9 (84%) | 20±6.1 (84%) | 9±0.3 (90%) | 14±1.0 (89%) | 15±0.8 (87%) | 5±0.4 (94%) | 7±0.4 (94%) | 9±0.7 (93%) | 13±0.7 (87%) |
| 90D | 16±1.6 (87%) | 17±0.6 (86%) | 3±0.1 (97%) | N/A | N/A | N/A | N/A | N/A | N/A |

N/A: Not available
*SS15 28D and 90D (n=3)

[symbol]

TABLE 4E

In Vivo Apixaban concentration (ng/mg) in the stented segment tissue and adjacent segments of 5 mm proximal and 5 mm distal to the implanted device
Apixaban in treated area tissue c ontent, ng/mg

| Stent (Sample Size) | 1 hour | 3 hours | 1 day | 6 days | 7 days | 28 days | 90 days |
|---|---|---|---|---|---|---|---|
| SStS(n=5) | 66.94 ± 27.33 | 25.31 ± 11.21 | 13.25 ± 10.17 | N/A | 1.15 ± 0.52 | 1.28 ± 0.47(n=3) | 3.05 ± 1.77(n=3) |
| Apixaban in Proximal tissue content, ng/mg | | | | | | | |
| Stent (Sample Size) | 1 hour | 3 hours | 1 day | 6 days | 7 days | 28 days | 90 days |
| SS15 (n=5) | 7.86 ± 3.01 | 5.15 ± 1.59 | 0.14 ± 0.08 | N/A | 0.01 ± 0.01 | 0.04(n=1) BQL(n=2) | 0.0003 ± 0.0002(n=2) |
| Apixaban in Distal tissue content, ng/mg | | | | | | | |
| Stent (Sample Size) | 1 hour | 3 hours | 1 day | 6 days | 7 days | 28 days | 90 days |
| SS15(n=5) | 3.53 ± 1.95 | 6.77 ± 2.22 | 0.36 ± 0.29 | N/A | 0.01 ± 0.01 | 0.02(n=1) BQL(n=2) | 0.0002± 0.0001(n=3) |

BQL: Below Quantification Limit

TABLE 4F

In Vivo Rivaroxaban concentration (ng/mg) in the stented segment tissue and adjacent segments of 5 mm proximal and 5 mm distal to the implanted device
Rivaroxaban in treated area tissue content, ng/mg

| Stent (Sample Size) | 1 hour | 3 hours | 1 day | 7 days | 28 days |
|---|---|---|---|---|---|
| SS16: Slider II Arm1 (n=5 except 3H n=4, 28d n=6) | 48.75±25.52 | 21.48± 5.80 | 3.67± 5.59 | 0.31 ± 0.24 | 0.34 ± 0.27 |
| SS17: Slider II Arm2 (n=5 except 3H n=4, 28d n=6) | 38.31± 16.08 | 26.23 ± 3.50 | 1.31 ± 0.28 | 1.07 ± 1.88 | 0.52 ± 0.63 |

Rivaroxaban in Proximal tissue content, ng/mg

| | | | | | |
|---|---|---|---|---|---|
| Stent (Sample Size) | 1 hour | 3 hours | 1 day | 7 days | 28 days |
| SS16: Slider II Arm1 (n=5 except 3H n=4, 28d n=6) | 2.63 ± 1.14 | 1.72 ± 0.53 | 0.09 ± 0.03 | BQL | 0.02 (n=1); BQL(n=5) |
| SS17: Slider II Arm2 (n=5 except 3H n=4, 28d n=6) | 1.52 ± 0.70 | 3.00 ± 1.50 | 0.09 ± 0.05 | 0.01(n=1); BQL(n=4) | 0.02 ± 0.001 (n=2); BQL(n=4) |

Rivaroxaban in Distal tissue content, ng/mg

| | | | | | |
|---|---|---|---|---|---|
| Stent (Sample Size) | 1 hour | 3 hours | 1 day | 7 days | 28 days |
| SS16: Slider II Arm1 (n=5 except 3H n=4, 28d n=6) | 6.51 ± 2.45 | 2.16± 0.83 | 0.09± 0.02 | BQL | 0.02(n=1); BQL(n=5) |
| SS17: Slider II Arm2 (n=5 except 3H n=4, 28d n=6) | 2.61 ± 1.38 | 2.72± 1.66 | 0.07± 0.02 | 0.01 (n=1); BQL (n=4) | BQL |

BQL: Below Quantification Limit
N/A: Not Available

TABLE 4G

In Vivo Argatroban concentration (ng/mg) in the stented segment tissue and adjacent tissue segment of 5 mm proximal and 5 mm distal to the implanted device
Argatroban in treated tissue content, ng/mg

| Stent (Sample Size) | 1 hour | 3 hours | 1 day | 6 days | 7 days | 28 days | 90 days |
|---|---|---|---|---|---|---|---|
| SS7 (n=1) | N/A | 54.00 | 0.75 | 0.09 | 0.05 | 0.15 | N/A |
| SS9 (n=1) | N/A | 123.10 | 0.18 | 0.33 | 2.54 | 1.76 | N/A |
| SS15 (n=5) | 71.37 ± 31.32 | 27.65 ± 15.00 | 15.64 ± 12.08 | N/A | 1.41 ±0.69 | 1.69 ± 0.64(n=3) | 3.74± 1.89(n=3) |
| SS16: Slider II Arm1 (n=5 except 3H n=4, 28d n=6) | 61.87 ± 24.60 | 32.81± 10.96 | 3.80± 4.87 | N/A | 0.42 ± 0.32 | 0.52 ± 0.37 | N/A |
| SS17: Slider II Arm2 (n=5 except 3H n=4, 28d n=6) | 11.80±2.69 | 8.06± 3.48 | 1.19±0.46 | N/A | 1.35±2.41 | 0.67±0.87 | N/A |
| Argatroban in Proximal tissue content, ng/mg | | | | | | | |
| Stent (Sample Size) | 1 hour | 3 hours | 1 day | 6 days | 7 days | 28 days | 90 days |
| SS7 (n=1) | N/A | 13.39 | BQL | BQL | BQL | BQL | N/A |
| SS9 (n=1) | N/A | 1.27 | BQL | BQL | BQL | BQL | N/A |
| SS15(n=5) | 8.57 ±3.66 | 5.43 ±2.67 | 0.05 ±0.03 | N/A | 0.01 ±0.01 | 0.04(n=1) BQL(n=2) | 0.0008 (n=1) BQL (n=2) |
| SS16: Slider II Arm1 (n=5 except 3H n=4, 28d n=6) | 3.74 ± 1.93 | 2.48 ±0.36 | 0.07 ±0.03 | N/A | BQL | BQL | N/A |
| SS17: Slider II Arm2 (n=5 except 3H n=4, 28d n=6) | 0.65 ±0.23 | 0.68 ±0.28 | 0.04 ±0.01 | N/A | BQL | BQL | N/A |
| Argatroban in Distal tissue content, ng/mg | | | | | | | |
| Stent (Sample Size) | 1 hour | 3 hours | 1 day | 6 days | 7 days | 28 days | 90 days |
| SS7 (n=1) | N/A | 5.20 | BQL | BQL | BQL | BQL | N/A |
| SS9 (n=1) | N/A | 11.33 | BQL | N/A | BQL | BQL | N/A |
| SS15 (n=5) | 3.84 ±2.42 | 6.46 ±3.14 | 0.17 ±0.09 | N/A | 0.01 ± 0.003 | 0.02(n=1) BQL(n=2) | 0.0006 ± 0.0004(n=2) BQL (n=1) |
| SS16: Slider II Arm1 (n=5 except 3H n=4, 28d n=6) | 7.87±3.04 | 2.63±0.87 | 0.09±0.03 | N/A | BQL | BQL | N/A |
| SS17: Slider II Arm2 (n=5 except 3H n=4, 28d n=6) | 0.91±0.51 | 0.87 ±0.52 | 0.03 ±0.01 | N/A | BQL | BQL | N/A |

BQL: Below Quantification Limit N/A: Not available

TABLE 4H

In Vivo Rapamycin concentration (ng/mg) in the tissue of treated segment (stented segment) and adjacent tissue segment of 5 mm proximal and 5 mm distal to the implanted device
Sirolimus in treated tissue content, ng/mg

| Stent (Sample Size) | 1 hour | 3 hours | 1 day | 6 days | 7 days | 28 days | 90 days |
|---|---|---|---|---|---|---|---|
| SS7 (n=1) | N/A | 7.34 | 4.46 | 1.38 | 0.73 | 1.79 | N/A |
| SS9 (n=1) | N/A | 40.57 | 0.93 | 1.61 | 3.24 | 2.48 | N/A |
| SS15 (n=5) | 43.22 ± 14.73 | 23.37 ± 6.88 | 29.17 ± 18.65 | N/A | 1.54 ± 0.35 | 1.67 ± 0.22(n=3) | 1.28 ± 0.07(n=3) |
| SS16: Slider II Arm1 (n=5 except 3H n=4, 28d n=6) | 45.10 ± 14.77 | 38.30 ± 9.29 | 9.18 ± 5.69 | N/A | 1.46 ± 0.38 | 0.94 ± 0.19 | N/A |
| SS17: Slider II Arm2 (n=5 except 3H n=4, 28d n=6) | 21.34 ± 7.51 | 27.32 ± 6.86 | 10.85 ± 3.55 | N/A | 3.80 ± 4.86 | 1.73 ± 1.52 | N/A |
| Sirolimus in Proximal tissue content, ng/mg | | | | | | | |
| Stent (Sample Size) | 1 hour | 3 hours | 1 day | 6 days | 7 days | 28 days | 90 days |
| SS7 (n=1) | N/A | 3.21 | BQL | 0.14 | BQL | 0.01 | N/A |
| SS9 (n=1) | N/A | 0.38 | 0.58 | BQL | 0.15 | 0.1 | N/A |
| SS15 (n=5) | 2.82 ± 1.15 | 3.52 ± 0.51 | 0.12 ± 0.11 | N/A | 0.02 ± 0.01 | 0.02 ± 0.01(n=3) | 0.01 ± 0.01(n=3) |
| SS16: Slider II Arm1 (n=5 except 3H n=4, 28d n=6) | 2.30 ± 1.22 | 2.62 ± 0.82 | 0.18 ± 0.06 | N/A | 0.04 ± 0.03 | 0.05 ± 0.05 (n=4); BQL(n=2) | N/A |
| SS17: Slider II Arm2 (n=5 except 3H n=4, 28d n=6) | 1.39 ± 0.83 | 2.49 ± 1.02 | 0.26 ± 0.27 | N/A | 0.01(n=3); BQL(n=2) | 0.03 ± 0.02 (n=4); BQL(n=2) | N/A |
| Sirolimus in Distal tissue content, ng/mg | | | | | | | |
| Stent (Sample Size) | 1 hour | 3 hours | 1 day | 6 days | 7 days | 28 days | 90 days |
| SS7 (n=1) | N/A | 1.86 | 0.99 | 0.69 | 0.46 | 0.14 | N/A |
| SS9 (n=1) | N/A | 6.56 | 1.88 | 0.39 | 0.61 | 0.21 | N/A |
| SS15 (n=5) | 1.66 ± 0.78 | 3.81 ± 0.61 | 3.18 ±3.01 | N/A | 0.27 ± 0.11 | 0.14 ± 0.04(n=3) | 0.02 ± 0.01(n=3) |
| SS16: Slider II Arm1 (n=5 except 3H n=4, 28d n=6) | 5.25 ± 1.62 | 3.78 ± 0.81 | 1.10 ± 0.44 | N/A | 0.55± 0.22 | 0.11 ± 0.05 | N/A |
| SS17: Slider II Arm2 (n=5 except 3H n=4, 28d n=6) | 1.81 ± 0.83 | 3.95 ± 3.09 | 0.86 ± 0.48 | N/A | 0.34 ± 0.21 | 0.13 ± 0.06 | N/A |

BQL: Below Quantification Limit
N/A: Not available

TABLE 4I

Tissue concentration of Rivaroxaban, Argatroban and Sirolimus number of orders of magnitude higher than IC50 for Anti-factor Xa/IIa or anti- cell proliferation for the respective drugs in the tissue of treated segment and adjacent tissue segment of 5 mm proximal and 5 mm distal to the implanted device for SS15

| Ratio of Tissue content to IC50 | 1 Hour | | | 3 Hours | | | 1 Day | | | 7 Days | | | 28 dyas | | | 3 month | | |
|---|---|---|---|---|---|---|---|---|---|---|---|---|---|---|---|---|---|---|
| | Proximal | Treated | Distal | Proximal | Treated | Distal | Proximal | Treated | Distal | Proximal | Treated | Distal | Proximal | Treated | Distal | Proximal | Treated | Distal |
| Apixaban | 215142 | 1821895 | 95316 | 141612 | 688998 | 185185 | 3813 | 362200 | 9804 | 272 | 31318 | 272 | 1089 | 34858 | 545 | 8 | 83061 | 5 |
| Argatroban | 806 | 6693 | 356 | 506 | 2587 | 609 | 5 | 1462 | 16 | 1 | 132 | 1 | 4 | 158 | 2 | 0 | 351 | 0 |
| Sirolimus | 28000 | 432000 | 17000 | 35000 | 234000 | 38000 | 1200 | 292000 | 31800 | 200 | 15400 | 2700 | 200 | 16700 | 1400 | 100 | 12800 | 200 |

*IC50 of Apixaban for anti-Xa 0.08 nM or 0.00004 ng/mg
*IC50 of Argatroban for anti-IIa 21 nM or 0.01ng/mg
*IC50 of Sirolimus for cell proliferation 0.1 nM or 0.0001 ng/mg

TABLE 4J

Tissue concentration of Rivaroxaban, Argatroban and Sirolimus number of orders of magnitude higher than IC50 for Anti-factor Xa/IIa or anti- cell proliferation for the respective drugs in the tissue of treated segment and adjacent tissue segment of 5 mm proximal and 5 mm distal to the implanted device for Slider II Arm1

| Ratio of Tissue content to IC50 | 1 Hour | | | 3 Hours | | | 1 Day | | | 7 Days | | | 28 days | | |
|---|---|---|---|---|---|---|---|---|---|---|---|---|---|---|---|
| | Proximal | Treated | Distal | Proximal | Treated | Distal | Proximal | Treated | Distal | Proximal | Treated | Distal | Proximal | Treated | Distal |
| Rivaroxaban | 286 | 5299 | 708 | 187 | 2335 | 235 | 10 | 399 | 10 | N/A | 34 | N/A | 2 | 37 | 2 |
| Argatroban | 351 | 5800 | 738 | 232 | 3076 | 247 | 7 | 356 | 8 | N/A | 39 | N/A | N/A | 49 | N/A |
| Rapamycin | 23000 | 451000 | 52500 | 26200 | 383000 | 37800 | 1800 | 91800 | 11000 | 400 | 14600 | 5500 | 500 | 9400 | 1100 |

*IC50 of Rivaroxaban for anti-Xa 21 nM or 0.0092 ng/mg
*IC50 of Argatroban for anti-IIa 21 nM or 0.01ng/mg
*IC50 of Sirolimus for cell proliferation 0.1 nM or 0.0001 ng/mg

TABLE 4K

Tissue concentration of Rivaroxaban, Argatroban and Sirolimus number of orders of magnitude higher than IC50 for Anti-factor Xa/IIa or anti- cell proliferation for the respective drugs in the tissue of treated segment and adjacent tissue segment of 5 mm proximal and 5 mm distal to the implanted device for Slider II Arm2

| Ratio of Tissue content to IC50 | 1 Hour | | | 3 Hours | | | 1 Day | | | 7 Days | | | 28 days | | |
|---|---|---|---|---|---|---|---|---|---|---|---|---|---|---|---|
| | Proximal | Treated | Distal | Proximal | Treated | Distal | Proximal | Treated | Distal | Proximal | Treated | Distal | Proximal | Treated | Distal |
| Rivaroxaban | 165 | 4164 | 284 | 326 | 2851 | 296 | 10 | 142 | 8 | 1 | 116 | 1 | 2 | 57 | N/A |
| Argatroban | 61 | 1106 | 85 | 64 | 756 | 82 | 4 | 112 | 3 | N/A | 127 | N/A | N/A | 63 | N/A |
| Rapamycin | 13900 | 213400 | 18100 | 24900 | 273200 | 39500 | 2600 | 108500 | 8600 | 100 | 38000 | 3400 | 300 | 17300 | 1300 |

IC50 of Rivaroxaban for anti-Xa 21 nM or 0.0092 ng/mg
IC50 of Argatroban for anti-IIa 21 nM or 0.01 ng/mg
IC50 of Sirolimus for cell proliferation 0.1 nM or 0.0001 ng/mg

Tables 4D-4K show that all three drugs Apixaban, Argatroban, and rapamycin maintain therapeutic tissue concentrations in the tissue segment up to 28 days, up to 90 days or longer, and furthermore achieve therapeutic tissue concentration in the adjacent tissue segment (±5 mm from the tissue segment such as Proximal and distal) at 1 hour, 3 hours and at/or up to 1 day. This can be important to inhibit thrombus formation in the stented segment, the device surface, and in the tissue adjacent to the stented segment as in many cases such tissue is injured by balloon deployment or stent edges.

Taking Apixaban IC50 for factor Xa inhibition to be about 0.08 nM or 0.00004 ng/mg; Rivaroxaban IC50 for factor Xa inhibition to be about 21 nM or 0.0092 ng/mg; Argatroban IC50 for factor IIa inhibition to be about 21 nM or 0.01 ng/mg; Sirolimus IC50 for cell proliferation to be about 0.1 nM or 0.0001 ng/mg. Table 4I-Table 4 K are the tissue concentration of Apixaban or Rivaroxaban, Argatroban and Sirolimus are several order of magnitude higher (or times higher) than IC50 for Anti-factor Xa, anti-IIa, or anti- cell proliferation for the respective drugs in the tissue of treated segment and adjacent tissue segment of 5 mm proximal and 5 mm distal to the implanted device. It shows that Apixaban, Rivaroxaban, Argatroban and/or Sirolimus in tissue concentrations have one or more order of magnitudes higher concentration at the times specified, has from 1 to 6 orders of magnitude of tissue concentration for each of the drugs compared to their IC50, in the treated tissue segments up to 28 days, or up to 90 days.

### Example 5: In Vivo Animal Study of Anticoagulant1/Anticoagulant2/mTOR Eluting Stents (Scaffolds)

The test drug eluting stent systems containing anticoagulants were prepared as described in Example 4 and were evaluated at 28 days and 90 days following implantation in a porcine coronary artery. The control device was the Novolimus (m-TOR) eluting DESyne X2 stent.

The porcine artery was chosen as this model has been used extensively for stent and angioplasty studies resulting in a large volume of data on the pulmonary response properties and its correlation to human pulmonary response (Schwartz et al, Circulation. 2002; 106:1867 1873). The animals were housed and cared for in accordance with the Guide for the Care and Use of Laboratory Animals as established by the National Research Council.

All animals were pretreated with aspirin (325 mg) and Clopidogrel (75 mg) per oral dose beginning at least 3 days prior to the intervention and continuing for the duration of the study. After induction of anesthesia, the left or right femoral artery was accessed using standard techniques and an arterial sheath was introduced and advanced into the artery. Vessel angiography was performed under fluoroscopic guidance, a 7 Fr. guide catheter was inserted through the sheath and advanced to the appropriate location where intracoronary nitroglycerin was administered. An appropriate implantation segment of coronary artery was randomly selected and a 0.014” guidewire inserted. Quantitative Coronary Angiography (QCA) was performed to document the results. The appropriately sized stent (3.0 × 14 mm or 3.5 × 14 mm) was advanced to the deployment site. The balloon was inflated at a steady rate to a pressure sufficient to achieve a balloon to artery ratio of approximately 1.1 to 1.0 but less than 1:2:1. Pressure was maintained for approximately 10 seconds before the balloon was deflated. Each pig was implanted with 3 test devices and one control device in the coronary arteries. Each time point a whole blood was drawn from animals for blood drug concentration test.

Follow up angiography imaging was performed at the designated endpoint for each of the animals. Quantitative coronary angiographic analysis was performed and the average percent diameter stenosis values and late lumen loss for the test arms and control DESyne X2 for the 28 days and 3-month time points are shown in Table 5A.

Upon completion of follow-up angiography imaging, the animals were euthanized. The hearts were harvested from each animal. Any myocardial lesions or unusual observations were reported. The coronary arteries were perfused with 10% buffered formalin at 100 to 120 mm Hg with the animal’s ear tag until processed for histology.

Stented portions of coronary arteries were embedded in methyl methacrylate (MMA), then divided into a target of three blocks of approximately similar length (about 4 mm), identified as proximal, mid and distal segments. From three blocks, 3 to 5 cuts were made for histology evaluation.

Quantitative histopathological evaluation of stented artery sections was then performed and scored as indicated. The mean of each section was recorded and then averaged to provide a mean score per stent for the different parameters (Table 5A). The smaller the score, the better the efficacy.

### Fibrin (Strut-by-Strut)

0 = absent, or rare minimal spotting around struts
1 = fibrin in small amounts, localized only around struts
2 = fibrin moderately abundant or denser, extending beyond struts
3 = abundant, dense fibrin, bridging between struts
Each strut in the section was scored; the mean fibrin score for each section was calculated and reported. The mean of the section means was calculated and reported, providing a mean fibrin score per stent.

### Injury Based on Schwartz Et Al. J Am Coll Cardiol 1992; 19:267-274. (Strut-by-Strut)

0 = IEL intact
1 = IEL lacerated
2 = media completely lacerated
3 = EEL lacerated
Each strut in the section as scored and the mean injury score for each section was calculated and reported. The mean of the section means was calculated and reported, providing a mean injury score per stent.

### Inflammation (Strut-by-Strut)

0 = no or very few (≤3) inflammatory cells around strut
1 = few (~4-10) inflammatory cells around strut
2 = many (> 10) inflammatory cells around strut, can extend into but does not efface surrounding tissue
3 = many (> 10) inflammatory cells, effacing surrounding tissue
Each strut in the section was scored and the mean inflammation score for each section was calculated and reported. The mean of the section means was calculated and reported, providing a mean inflammation score per device.

TABLE 5A

Histopathology Scores and Quantitative Coronary Angiography data Apixaban/Rivaroxaban, Argatroban and Sirolimus releasing 14 mm stents at 7 days, 28 days and 3 month

| Time Point | Device | Injury | Inflammation | Fibrin | Diameter Stenosis% | LLL, mm |
|---|---|---|---|---|---|---|
| 7 Day | SS7(n=1) | 0.15 | 1.52 | 0.62 | N/A | N/A |
| | SS9(n=1) | 0.61 | 1.66 | 0.44 | N/A | N/A |
| 28 Day | SS7(n=1) | 0.21 | 0.49 | 0.94 | 16.7 | 0.71 |
| | SS9(n=1) | 0.36 | 0.32 | 0.64 | 21.3 | 0.76 |
| | SS15 (n=3) | 0.38±0.34 | 0.62±0.12 | 1.67±0.38 | 19.8±4.1 | 0.45±0.15 |
| | DESyne X2(n=1) | 1.34 | 1.83 | 1.87 | 51.6 | 1.14 |
| | SS16(Slider II Arm1, n=6) | 0.91± 0.95 | 1.52± 1.03 | 1.90± 0.26 | 19.4 ± 13.8 | 0.72± 0.32 |
| | DESyne X2(n=2) | 1.17 ± 1.60 | 1.97 ± 1.46 | 1.32 ± 1.11 | 36.6 ± 27.3 | 0.99 ± 0.48 |
| | SS17(Slider II Arm2, n=6) | 1.01±0.47 | 1.46±0.43 | 1.88±0.48 | 33.1±12.8 | 0.86±0.34 |
| | DESyne X2 (n=2) | 0.87±0.74 | 1.59±0.95 | 1.93 ±0.31 | 21.9± 11.6 | 0.71 ±0.62 |
| 3 Month | SS15 (n=3) | 0.15± 0.18 | 0.52±0.19 | 0.08± 0.06 | 18.2± 9.3 | 0.25 ± 0.29 |
| | DESyne X2(n=1) | 0.43 | 0.59 | 0.76 | 22.3 | 0.52 |

LLL is an indicator of the amount cell proliferation or inhibition potency. It is used to measure efficacy between drugs for proliferation inhibition in mammalian arteries. The smaller the LLL, the better the efficacy of the drug.

As shown in Table 5A, SS 15 composition providing the combination of Sirolimus, Apixaban and Argatroban released from stents had a smaller LLL compared to control which only had m-TOR inhibitor (Novolimus) and thus was unexpectedly more effective at inhibiting smooth muscle cell proliferation compared to Novolimus releasing stents at 28 days, and at 90 days. This was an unexpected finding for the test SS15 stents in comparison to the control DESyne X2 stents at the 28-day time point and/or at 90 days.

In an unexpected finding, SS15 stents composition eluting Apixaban, Argatroban, and the M-Tor inhibitor rapamycin exhibited more efficacy at inhibiting one or more of the following at 28 days and/or 90 day time points: cell proliferation, inflammation, injury, fibrin formation inhibition, and fibrin dissolution acceleration.

The LLL is an indicator of the amount cell proliferation or inhibition potency. It is used to measure efficacy between drugs for proliferation inhibition in mammalian arteries. The smaller the LLL, the better the efficacy of the drug.

As shown in Table 5A, SS16 shows the combination of Sirolimus, Rivaroxaban and Argatroban released from stents had a smaller LLL compared to control which only had m-TOR inhibitor (Novolimus) and thus was unexpectedly more effective at inhibiting smooth muscle cell proliferation compared to Novolimus releasing stents at 28 days.

As shown in Table 5A, SS17 composition configured to delay the release and tissue concentration of rapamycin within the first 1 hour and/or within the first 3 hours by incorporating rapamycin in the base coating shows the combination of Sirolimus, and/or lower tissue concentration of Rivaroxaban and Argatroban within at least the first hour showed less inhibition of SMC proliferation at 28 days.

TABLE 5B

Whole Blood PK Results of Apixaban/Argatroban/Sirolimus Eluting Stents from SS15(from study with SS15; target dose Apixaban/Argatroban/Sirolimus=117/117/94; n=5)

| Time Points | Apixaban (ng/mL) | | | | | Argatroban (ng/mL) | | | | | Sirolimus (ng/mL) | | | | |
|---|---|---|---|---|---|---|---|---|---|---|---|---|---|---|---|
| | 1 H | 3 H | 1 D | 7 D | 28D | 1 H | 3 H | 1 D | 7 D | 28D | 1 H | 3 H | 1 D | 7 D | 28D |
| Pre-implant | BQL | BQL | BQL | BQL | BQL | BQL | BQL | BQL | BQL | BQL | BQL | BQL | BQL | BQL | BQL |
| Post 1^{st} implant | 0.44 1 | 0.71 | 4.02 | 0.16 | 0.30 | 1.36 | 2.02 | 4.44 | 0.58 | 0.66 | 0.70 | 0.59 | 2.52 | 0.69 | 0.32 |
| Post 2^{nd} implant | 0.95 | 1.17 | 3.51 | 0.77 | 0.44 | 3.16 | 4.35 | 5.74 | 4.41 | 1.83 | 1.90 | 1.94 | 4.16 | 2.13 | 1.06 |
| Post 3^{rd} implant | 1.8 | 2.18 | 4.29 | 1.31 | 0.91 | 6.71 | 7.59 | 9.26 | 7.85 | 3.72 | 3.30 | 3.74 | 7.08 | 3.28 | 2.49 |
| Post 4^{th} implant | 2.56 | 2.91 | 5.01 | 1.72 | 1.65 | 10.2 | 11.0 | 11.4 | 10.9 | 7.33 | 4.80 | 5.22 | 8.06 | 4.17 | 4.73 |
| Post 5^{th} implant | 3.27 | 3.55 | 5.84 | 2.12 | 2.45 | 13.4 | 13.3 | 14.5 | 12.8 | 10.3 | 6.71 | 7.13 | 9.98 | 4.75 | 5.91 |
| 15 min. | 3.91 | 4.60 | ------ | ------ | ------ | 17.8 | 21.7 | ------ | ------ | --- | 8.77 | 11.3 | ----- | ------ | ------ |
| 30 min. | 4.78 | 5.20 | 8.69 | ----- | ----- | 22.7 | 24.6 | 22.6 | ------ | --- | 11.2 | 12.3 | 17.2 | ------ | ------ |
| 45 min. | 6.29 | ------ | ------ | ------ | ------ | 28.9 | ------ | ----- | ------ | ---- | 13.4 | ------ | ----- | ------ | ------ |
| 60 min. (1 hr) | 6.32 | 6.28 | 9.91 | ------ | ------ | 28.2 | 29.0 | 24.4 | ----- | ---- | 12.8 | 12.7 | 21.3 | ------ | ------ |
| 90 min. | ------ | 7.26 | ------ | ------ | ------ | ------ | 31.1 | ------ | ------ | ----- | ------ | 13.7 | ------ | ----- | ------ |
| 120 min.(2 hr) | ------ | 7.76 | 12.7 | ------ | ------ | ------ | 28.5 | 20.7 | ------ | ----- | ------ | 12.8 | 21.3 | ------ | ------ |
| 150 min. | ------ | 7.38 | ------ | ------ | ------ | ------ | 28.9 | ------ | ------ | ----- | ------ | 12.4 | ---- | ------ | ------ |
| 180 min.(3 hr) | ------ | 6.89 | 12.8 | ------ | ------ | ------ | 20.4 | 16.9 | ------ | ----- | ------ | 9.32 | 14.0 | ------ | ------ |
| 4 hr | ------ | ------ | 13.3 | ------ | ------ | ------ | ------ | 12.9 | ------ | ----- | ------ | ------ | 12.4 | ------ | ------ |
| 5 hr | ----- | ------ | 14.7 | ------ | ------ | ------ | ------ | 9.25 | ------ | ----- | ------ | ------ | 11.0 | ------ | ------ |
| 6 hr | ------ | ------ | 13.3 | ------ | ------ | ------ | ------ | 7.02 | ------ | ----- | ----- | ------ | 10.5 | ------ | ------ |
| 24 hr(Day 1) | ------ | ------ | 1.51 | ------ | ------ | ------ | ------ | 0.38 3 | ------ | ----- | ------ | ------ | 2.65 | ------ | ------ |
| Day 7 | - | - | - | BQL | - | - | - | - | BQL | - | - | - | - | 0.38 4 | - |
| Day 28 | - | - | - | - | BQL | - | - | - | - | BQL | - | - | - | - | BQL |

Note. Blood volume in porcine model is about 40%-50% of adult human. Thus drug concentrations in human would typically be lower than the figures shown in table 5B.

TABLE 5C

Whole Blood PK Results of Rivaroxaban /Argatroban/Sirolimus Eluting Stents from SS16 (Rivaroxaban Arm1; target dose Rivaroxaban/Argatroban/Sirolimus=117/117/94; n=5 except D28 n= 6)

| Time Points | Rivaroxaban (ng/mL) | | | | Argatroban (ng/mL) Sirolimus (ng/mL) | | | | | | | |
|---|---|---|---|---|---|---|---|---|---|---|---|---|
| | 1 H | 1 D | 7 D | 28 D | 1 H | 1 D | 7 D | 28 D | 1 H | 1 D | 7 D | 28 D |
| Pre-implant | BQL | BQL | BQL | BQL | BQL | BQL | BQL | BQL | BQL | BQL | BQL | BQL |
| Post 1^{st} implant | 0.72 | 1.14 | 1.07 | 0.69 | 1.96 | 2.94 | 2.22 | 1.89 | 0.52 | 0.69 | 0.66 | 0.48 |
| Post 2^{nd} implant | 1.34 | 2.16 | 2.14 | 1.44 | 5.05 | 6.92 | 5.91 | 4.76 | 1.56 | 1.47 | 1.75 | 1.29 |
| Post 3^{rd} implant | 2.10 | 2.57 | 2.94 | 2.17 | 10.3 | 10.1 | 10.2 | 8.22 | 3.53 | 2.22 | 3.35 | 2.32 |
| Post 4^{th} implant | 2.67 | 3.36 | 3.52 | 2.80 | 15.0 | 17.2 | 13.6 | 11.69 | 4.69 | 3.41 | 4.69 | 3.59 |
| Post 5^{th} implant | 2.92 | 4.32 | 3.95 | 3.33 | 18.9 | 19.9 | 19.1 | 14.42 | 6.15 | 5.37 | 7.24 | 5.08 |
| Post 6^{th} implant | - | - | - | 4.32 | - | - | - | 19.05 | - | - | - | 6.58 |
| 15 min. | 3.32 | 4.05 | - | - | 23.5 | 22.8 | - | - | 8.38 | 6.92 | - | - |
| 30 min. | 3.34 | 3.76 | - | - | 27.5 | 27.0 | - | - | 10.1 | 8.56 | - | - |
| 45 min. | 3.68 | 3.47 | - | - | 31.9 | 31.5 | - | - | 10.9 | 9.93 | - | - |
| 60 min. (lhr) | 4.08 | 2.61 | - | - | 31.1 | 31.5 | - | - | 11.2 | 10.7 | - | - |
| 90 min. | - | 2.43 | - | - | - | 30.8 | - | - | - | 12.3 | - | - |
| 120 min.(2 hr) | - | 2.30 | - | - | - | 26.6 | - | - | - | 12.1 | - | - |
| 150 min. | - | 2.08 | - | - | - | 26.6 | - | - | - | 11.7 | - | - |
| 180 min.(3 hr) | - | 1.95 | - | - | - | 25.1 | - | - | - | 10.3 | - | - |
| 4 hr | - | 1.23 | - | - | - | 18.4 | - | - | - | 7.62 | - | - |
| 5 hr | - | 1.11 | - | - | - | 13.5 | - | - | - | 6.89 | - | - |
| 6 hr | - | 1.03 | - | - | - | 11.0 | - | - | - | 6.09 | - | - |
| 24 hr(Day 1) | - | BQL | - | - | - | 1.92 | - | - | - | 2.14 | - | - |
| Day 7 | - | - | BQL | - | - | - | BQL | - | - | - | 0.44 | - |
| Day 28 | - | - | - | BQL | - | - | - | BQL | - | - | - | BQL |

Note. Blood volume in porcine model is about 40%-50% of adult human. Thus drug concentrations in human would typically be lower than the figures shown in table 5C.

TABLE 5D

Whole Blood PK Results of Rivaroxaban /Argatroban/Sirolimus Eluting Stents from SS17 Rivaroxaban/ Argatroban/Sirolimus= 117/117/94; n=5 except D28 n= 6)

| Time Points | Rivaroxaban (ng/mL) | | | | Argatroban (ng/mL) | | | | Sirolimus (ng/mL) | | | |
|---|---|---|---|---|---|---|---|---|---|---|---|---|
| | 1 H | 1 D | 7 D | 28 D | 1 H | 1 D | 7 D | 28 D | 1 H | 1 D | 7 D | 28 D |
| Pre-implant | BQL | BQL | BQL | BQL | BQL | BQL | BQL | BQL | BQL | BQL | BQL | BQL |
| Post 1^{st} implant | 3.77 | 3.62 | 3.18 | 3.20 | 11.7 | 11.1 | 11.0 | 14.05 | BQL | 0.11 | BQL | BQL |
| Post 2^{nd} implant | 5.27 | 6.16 | 5.90 | 5.79 | 20.5 | 24.7 | 25.9 | 32.44 | 0.17 | 0.36 | BQL | 0.06 |
| Post 3^{rd} implant | 9.27 | 11.4 | 8.41 | 8.66 | 41.3 | 45.2 | 44.3 | 52.20 | 0.28 | 0.44 | 0.22 | 0.21 |
| Post 4^{th} implant | 10.9 | 16.7 | 10.8 | 9.02 | 54.4 | 59.6 | 63.2 | 65.75 | 0.47 | 0.91 | 0.77 | 0.46 |
| Post 5^{th} implant | 12.5 | 22.3 | 12.0 | 10.70 | 65.7 | 70.3 | 73.6 | 79.38 | 0.98 | 1.38 | 1.08 | 0.69 |
| Post 6^{th} implant | ------ | ------ | ------ | 13.42 | ------ | ------ | ----- | 96.18 | ------ | ------ | ------ | 1.20 |
| 15 min. | 10.8 | 24.0 | ------ | ------ | 73.4 | 67.2 | ------ | ------ | 2.11 | 2.88 | ------ | ------ |
| 30 min. | 8.44 | 20.1 | ----- | ------ | 51.9 | 51.7 | ------ | ------ | 2.79 | 4.65 | ------ | ------ |
| 45 min. | 7.82 | 19.5 | ------ | ------ | 42.4 | 46.5 | ------ | ------ | 3.96 | 5.35 | ------ | ------ |
| 60 min. (1 hr) | 7.54 | 18.5 | ------ | ------ | 38.3 | 44.2 | ------ | ------ | 4.32 | 6.97 | ------ | ------ |
| 90 min. | ------ | 15.0 | ------ | ------ | ------ | 32.9 | ------ | ------ | ------ | 8.08 | ------ | ------ |
| 120 min.(2 hr) | ------ | 11.6 | ------ | ------ | ------ | 25.8 | ------ | ------ | ------ | 8.12 | ------ | ------ |
| 150 min. | ----- | 11.3 | ------ | ------ | ------ | 20.5 | ------ | ------ | ------ | 7.97 | ------ | ------ |
| 180 min.(3 hr) | ------ | 8.39 | ------ | ------ | ------ | 16.3 | ------ | ------ | ------ | 7.10 | ------ | ------ |
| 4 hr | ------ | 3.74 | ------ | ------ | ------ | 9.30 | ------ | ------ | ------ | 5.63 | ------ | ------ |
| 5 hr | ------ | 3.90 | ------ | ------ | ------ | 6.78 | ------ | ------ | ------ | 6.80 | ------ | ------ |
| 6 hr | ------ | 3.10 | ------ | ------ | ------ | 5.40 | ------ | ------ | ------ | 6.30 | ------ | ------ |
| 24 hr(Day 1) | ------ | 0.16 | ------ | ------ | ------ | 0.48 | ------ | ------ | ------ | 2.92 | ------ | ------ |
| Day 7 | ----- | ----- | BQL | ------ | ------ | ----- | 0.36 | ------ | ------ | ----- | 0.49 | ------ |
| Day 28 | ----- | ----- | ------ | BQL | ------ | ----- | ----- | BQL | ------ | ----- | ----- | 0.66 |

Note. Blood volume in porcine model is about 40%-50% of adult human. Thus drug concentrations in human would typically be lower than the figures shown in table 5D.

Tables 5B, 5C, and 5D show although local (tissue adjacent to the device) concentrations of Apixaban, Rivaroxaban, and Argatroban reached therapeutic levels, the systemic blood concentrations for each of the drugs were below one or more of the following to achieve systemic therapeutic concentrations: systemic Cmax, Systemic Cmean, Systemic Ctrough. These tables also show that each of these agents reached BQL levels within one of the following: 1 day, 7 days, 28 days, or 90 days

### Example 6: Anti-Proliferative Activity of Apixaban, Argatroban and Rapamycin Combination

Anti-proliferative activity of Apixaban, Argatroban, and Rapamycin was tested in Human Aortic SMC (HAoSMC, ATCC, PCS-100-012). Cell proliferation assay was done in 96-well format. Low passage cells were trypsinized and seeded in 96 well plates at a density of ~4000 cells/well. The cells are allowed to attach overnight in a CO₂ incubator. Next day, the medium was removed and replaced with fresh complete medium containing various concentrations of the test compounds. The final concentration of vehicle (DMSO) in the test medium was 0.1%. After adding test compounds, the cells were incubated for 72 hours. Following this period, the medium was removed and then added fresh medium (100 µl) containing CellTiter Aqueous (1x concentration final) to the wells and incubated for 2 hours in the CO2 incubator. At the end of incubation measured fluorescence with a plate-reader. Controlled incubations with untreated cells and blank incubations containing only medium were included and tested similarly. Based on the cell viability assay the percentage inhibition of the cell proliferation was determined at the different concentrations of the drug tested.

The cell proliferation assay was performed with different concentrations of Apixaban and Argatroban when combined with Rapamycin. Following the cell proliferation assay as indicated earlier, the percent cell proliferation inhibition was determined, and the assay results plotted to determine the IC50.

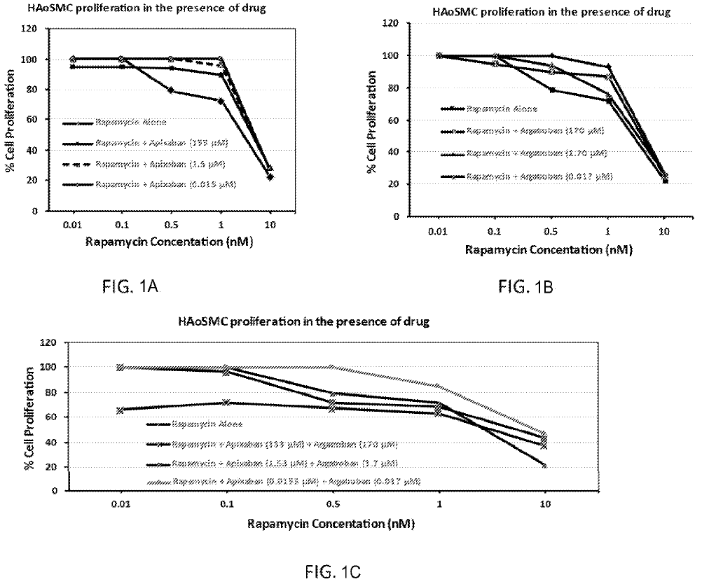

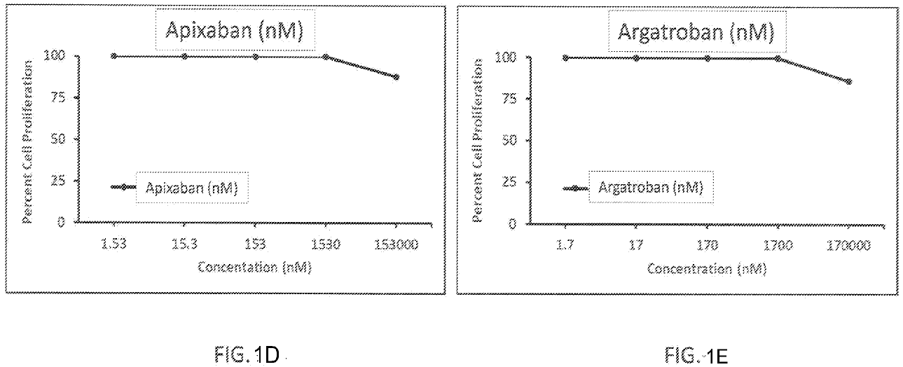

FIGS. 1A-1C show HAoSMC proliferation inhibition in the presence of different drug combinations. The data shows the combination of Apixaban, Argatroban surprisingly and unexpectedly enhanced the anti-proliferative effects of rapamycin on smooth muscle cell proliferation as measured by cell proliferation test when Apixaban and Argatroban were combined with rapamycin, i.e the combination of Apixaban, Argatroban, and rapamycin were more potent than rapamycin alone at inhibiting SMS proliferation.

FIGS. 1D and 1E show HAoSMC proliferation in presence of different concentrations of Apixaban or Argatroban. In order to determine if Apixaban or Argatroban independently had inhibitory effect on the proliferation of HAoSMC, a proliferation assay in the presence of either of these two drugs at different concentrations were tested as described earlier. Various concentration of Apixaban alone or Argatroban alone had small to no inhibition of HAoSMC proliferation was observed as shown in FIGS. 1D-1E.

### Example 7: Activated Clotting Time (ACT) Evaluation of Apixaban, Argatroban or a Combination of Apixaban and Argatroban

The activated clotting time (ACT) evaluation of anticoagulants was performed in Calcium-reconstituted sheep blood and recorded employing the Hemochron® Response device.

The ACT measurements were made in citrated sheep blood. 1.9 ml of citrated sheep blood was added to a test tube containing an activator (Hemochron@Celite@ ACT tubes, Lot F8FTE026 from Accriva Diagnostics, Inc.). A target amount of drug solution was then added into the test tube. The test tube was gently swirled so that the blood and drug was well-mixed. 0.1 ml of 0.3 M calcium chloride was then added. The tube was gently shaken before being inserted into the Hemochron Response detector. The ACT read out was recorded and reported. The ACT of the control blood in the absence of any drug as first determined to establish a baseline. Then ACT was determined in the presence of different concentrations of the drug as a single component. Selected drug combinations, were then tested to evaluate for potential synergy in action between the two drugs.

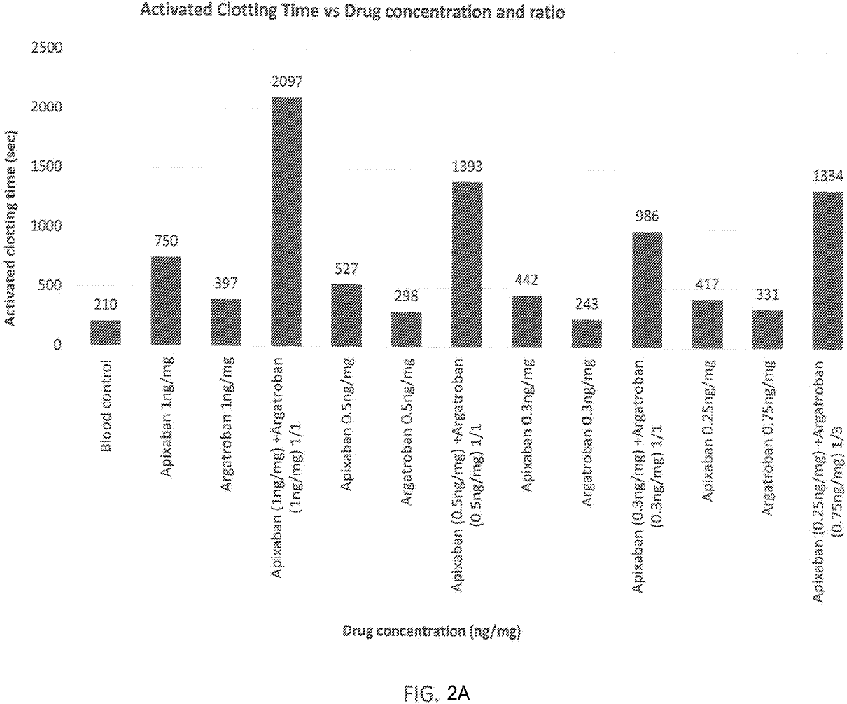

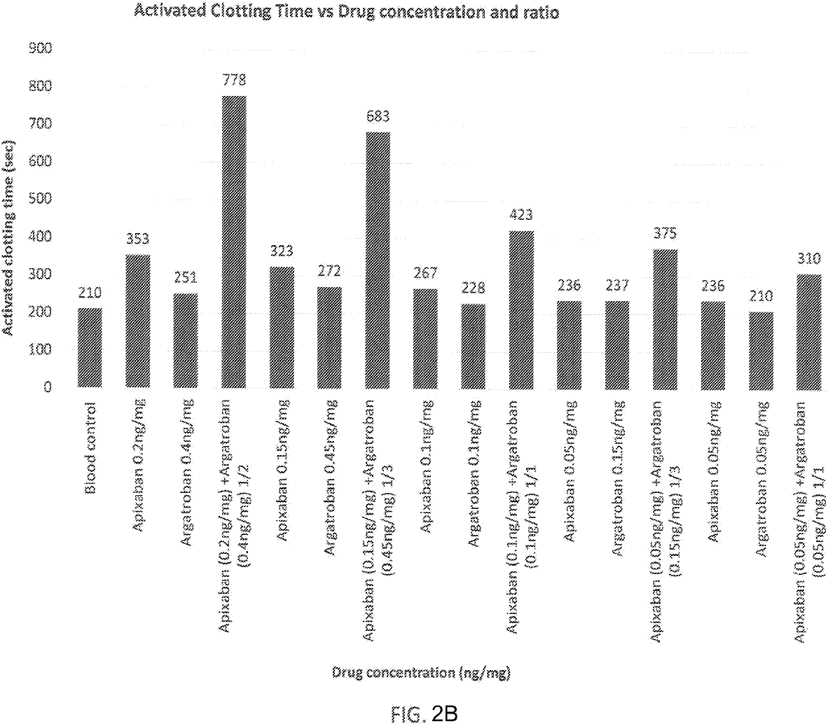

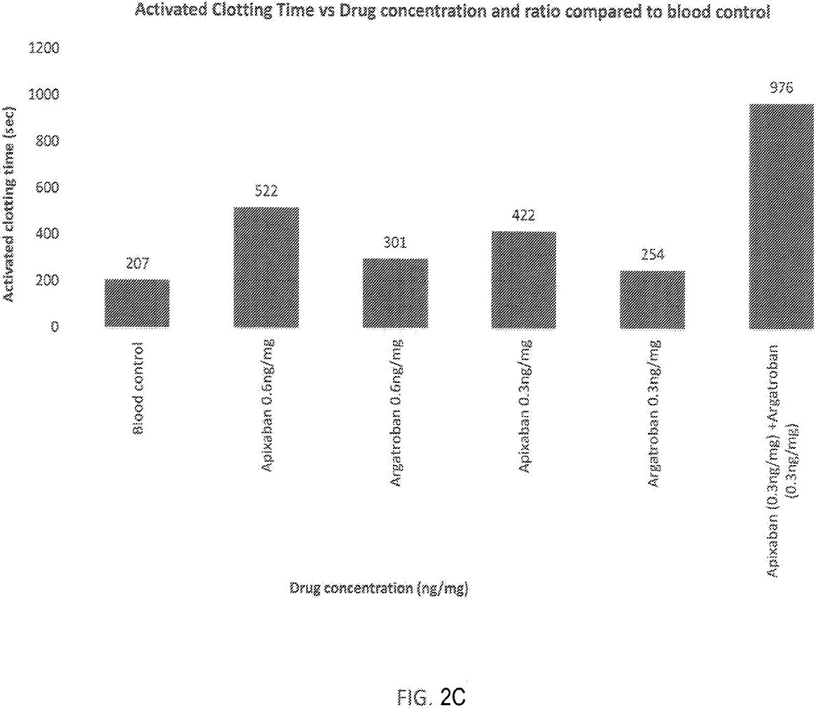

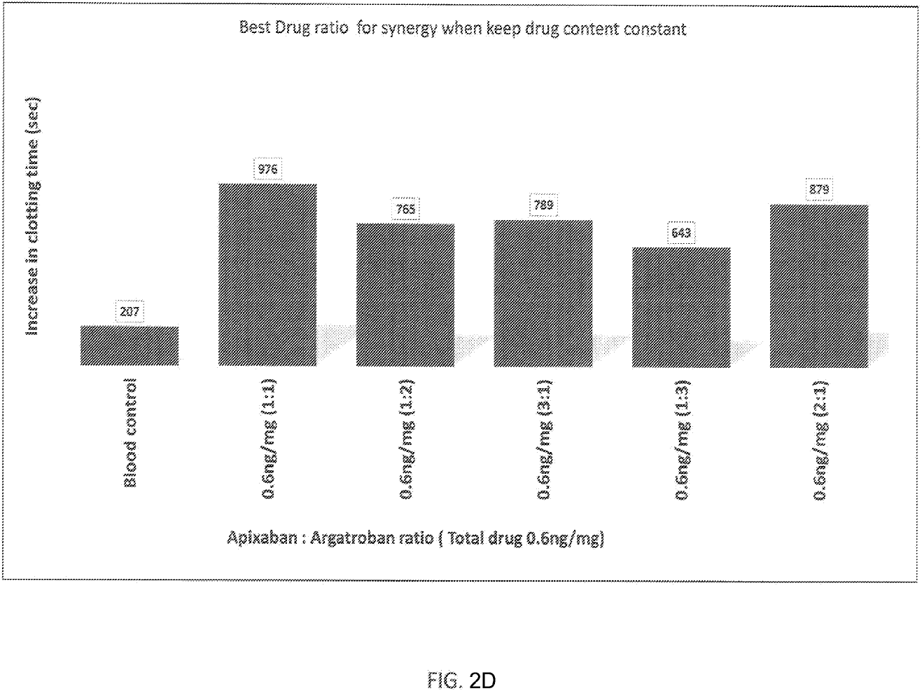

As shown in FIGS. 2A-2D, the clotting time was observed to be significantly extended or increased at a higher drug combination concentration. The Apixaban/Argatroban combination achieved ACT levels that were higher than the sum of the individual ACT values, indicating a synergistic effect between these drug combinations. This may be particularly important when delivering these drugs locally (adjacent to injured tissue) to inhibit clot formation. The figures are presented in ng/mg wherein the density of blood and tissue are approximately the same.

It was found, unexpectedly, that the combination drug concentrations of 0.025 ng/mg for each Apixaban and Argatroban drug extended the ACT by a larger time (as shown in FIG. 2B).

It was found, unexpectedly, that the combination drug concentrations of 0.3 ng/mg for each drug (0.6 ng/mg total) extended the ACT by a larger time (i.e., was more effective) than the ACT for each individual drug at 0.6 ng/mg concentration (ACT of 976 for the combination versus 522 for Apxaban versus 301 for Argatroban as shown in FIG. 2C).

FIG. 2C further shows, unexpectedly, that the combination drug concentrations of 0.3 ng/mg for each drug (0.6 ng/mg total) extended the ACT by a larger time (i.e., was more effective) than the ACT for the sum of each individual drug ACT at 0.3 ng/mg or at 0.6 ng/mg concentration. (ACT of 976 for the combination versus 676 (for the sum of individual drugs having 0.3 ng/mg concentrations).

It is important to note that drug tissue concentrations for factor Xa inhibitors like Rivaroxiban or Apixaban alone or in combination with factor IIa Argatrban to have sufficient tissue concentrations in the stented tissue segment and in the adjacent tissue segment to have therapeutic levels for each drug to be larger than 0.02 ng/mg, larger than 0.1 ng/mg, preferably larger than 0.2 ng/mg of tissue, preferably 0.3 ng/mg of tissue, more preferably larger than 1 ng/mg of tissue at or within 3 hours after implantation, or at or within 1 day after implantation, to inhibit clot formation.

It was also shown that the combination of Apixaban and Argatroban with m-TOR inhibitor inhibited thrombus formation in a shunt model (e.g., as in Example 8).

### Example 8: Ex Vivo Testing, of Drug, Eluting Stent Compared With 2 Anticoagulants and mTOR Eluting Stents

The thrombogenicity of a drug eluting stent system with two anticoagulant Apixaban and Argatroban in combination with rapamycin at two different loading drug doses was evaluated at 1 hour in an arteriovenous ex vivo shunt in a porcine model wherein the devices were deployed in a blood compatible polymeric tubing.

The control stents were 16-o-demethyl rapamycin m-TOR inhibitor (Novolimus) drug eluting coronary stent (DESyne, Elixir) and m-TOR inhibitor Zotarolimus eluting coronary stent (Resolute, Medtronic, USA).

The test arm for this experiment were SS9, SS9*, and SS10* and were manufactured as follows: Each polymer solution and each drug solutions were combined together ((Sirolimus and anticoagulant Apixaban and Argatroban was 1:1:1) to poly(L- lactide acid-co- glycolic acid) by weight ratio was 5:2 matrix) according to the target drug dose of 235 µg for each anticoagulant and 94 µg for Sirolimus for SS9, SS9* test arm was about ⅓ of each of the drugs dose as follows: Sirolimus and anticoagulant Apixaban and Argatroban was 1:1:1) to poly(L- lactide acid-co- glycolic acid) by weight ratio was 5:2 on matrix) according to the target drug dose of 78.3 µg for each anticoagulant and 31.3 µg for Sirolimus, and SS10* arm was Sirolimus and anticoagulant Apixaban and Argatroban was 1:1:3) to poly(L- lactide acid-co- glycolic acid) by weight ratio was 5:2 matrix) according to the target drug dose of 39.2 µg for Apixaban and 117.5 µg for Argatroban and 31.3 µg for Sirolimus.

A microprocessor controlled ultrasonic sprayer was used to coat each of the stents 14 mm length uniformly with each of the drug/polymer matrix solution. After coating, the stents were placed in a 70° C. oven for about 2 hours to remove the solvent. The stents were then mounted on balloon catheters and crimped. The catheters were then inserted in coils and packaged. The pouches were sterilized.

The ex-vivo shunt model to evaluate thrombogenicity has been extensively employed to evaluate the biocompatibility of different drug eluting stents (Waksman et al. Circ Cardiovasc Interv. 2017; 10:e004762, Otsuka et al. J Am Coll Cardiol Intv 2015; 8:1248-60, Lipinski et al. EuroInterv 2018; Jaa-369 2018) The animals were housed and cared for in accordance with the Guide for the Care and Use of Laboratory Animals as established by the National Research Council.

The two pigs that were employed in this study did not receive any aspirin or clopidogel pretreatment. Further all procedures were performed in the absence of any anticoagulant including heparin. After induction of anesthesia, an arterial bypass shunt from the femoral artery to the femoral vein was created.. Blood flow was established through the shunt. Flow rates through the shunt was continuously monitored during the procedure with a flow probe that was placed on the shunt tubing proximal to the arterial flow.

In the first pig three control devices were deployed in the first shunt and the blood flow through the shunt was performed for a period of 1 hour. Following perfusion, the shunt tubing containing the stents was rinsed with saline and then fixed in situ with 10% buffered formalin in order to capture the thrombus, if any, that are deposited on the stent surface.

Similar procedure with 3 shunts with only one stent SS9* or SS9 in each shunt was tested with a perfusion time of 1 hour for each of the shunts.

In the second pig three control devices were deployed in the first shunt and the blood flow through the shunt was performed for a period of 1 hour. Following perfusion, the shunt tubing containing the stents was rinsed with saline and then fixed in situ with 10% buffered formalin in order to capture the thrombus, if any, that are deposited on the stent surface.

Similar procedure with 2 shunts with only one stent SS10* in each shunt was tested with a perfusion time of 1 hour for each of the shunts.

Promptly following perfusion in each of the shunts, the tubing containing the stents was gently rinsed with saline under gravity flow and then fixed in situ with 10% buffered formalin in order to anchor the thrombus, if any, that are deposited on the stent surface.

The stents were then removed from the tubing and bisected longitudinally. Low magnification photographs of the luminal side of two halves of the control and test stents were recorded.

The two halves of the stents were then processed for scanning electron microscopy (SEM) so as to examine the thrombus on the luminal side of the stent. Low (15x) and high (200x) magnification images of the stent surface were captured to evaluate the extent of thrombus deposition on the luminal surface of the stent.

Significant number of thrombus was observed on the luminal surface (inner surface) of the control DES stents as seen on the low and high magnification SEM images whereas there was little or no thrombus deposits on the test stents with combinations of Apixaban and Argatroban and m-TOR. The number of thrombus deposits on the control and test stents as evaluated from SEM images are shown in Table 6. The data shows that the combinations of Apixaban and Argatroban and m-TOR inhibited thrombus formation in the shunt model better than control.

Table 6 shows several therapeutic compositions of factor Xa inhibitor, factor IIa, and M-tor inhibitor releasing stents had less thrombus (clot formation) compared to M-Tor inhibitor alone releasing stents.

The composition comprising a combination of factor Xa inhibitor, a factor II inhibitor and an anti-proliferative were surprisingly more effective than the anti-proliferative alone.

TABLE 6

Thrombus deposits on the control and test stents as evaluated from SEM images

| Animal # | Control/Test DES | Device | Number of Thrombus deposits |
|---|---|---|---|
| 1 | Control | DESyne-1 | 18 |
| | | DESyne-2 | 40 |
| | | Resolute | 14 |
| | Test - Apixaban:Argatroban(1:1) | SS9* | 4 |
| | | SS9* | 0 |
| | | SS9 | 3 |
| 2 | Control | DESyne-1 | 17 |
| | | DESyne-2 | 17 |
| | | Resolute | 15 |
| | Test - Apixaban: Argatroban(1:3) | SS10* | 2 |
| | | SS10* | 2 |

### Example 9 Preparation of Anticoagulant or mTOR Inhibitors Coated Balloon

Rivaroxaban,Apixaban, Novolimus, or Rapamycin were dissolved into dichloromethane at room temperature and vortex until the drug was uniformly dissolved/dispersed; Ethylene Vinyl Acetate / Polyvinylpyrrolidone (MW=1.3 M) was dissolved into 6.25% methanol in dichloromethane at room temperature and vortex until the polymer had uniformly dissolved/dispersed (100% Dichloromethane was used for Polyethylene oxide (MW=8 M)).

Each polymer solution and each drug solutions were combined together (Rivaroxaban to Ethylene vinyl acetate / Polyvinylpyrrolidone (MW=1.3 M) by weight ratio was 2:1:1 for SV300), (Apixaban to Polyethylene oxide (MW=8M)/ Polyvinylpyrrolidone (MW=1.3 M) / Butylated hydroxytoluene by weight ratio was 2/1/1/0.01 for VGR), (Novolimus to Ethylene vinyl acetate / Polyvinylpyrrolidone (MW=1.3 M) by weight ratio was 2:1:1 NEV250 and NEV200), and (Rapamycin to Ethylene vinyl acetate / Polyvinylpyrrolidone (MW=1.3 M) by weight ratio was 2:1:1 for REV200) according to the target drug dose of 300 µg for Rivaroxaban in SV300, 800 µg for Apixaban in VGR, 250 µg for Novoliums in NEV250, 200 µg for Novoliums in NEV200 and 200 µg for Rapamycin in REV200.

A microprocessor controlled ultrasonic sprayer was used to coat each of the balloon 14 mm or 18 mm length uniformly (as shown in Table 7) with each of the drug/polymer matrix solutions. Balloons were inflated prior to coating and held by a rotating fixture. A rotational motor rotated the catheter and balloon 360 degrees while a mandrel and a clamp securely held the catheter tail in place and rotate. The coating parameter was adjusted to ideal coating texture and the morphology and the profile of the interface between drug and balloon surface. After coating, the balloons were placed in a vacuum chamber to remove the solvent. The balloons were then tri-folded before putting on the protective sheath. The balloon catheters were then inserted in coils and packaged. The pouches were sterilized.

### Example 10: In Vivo Pharmacokinetics of Drug Eluting Balloon With Anticoagulant

The pharmacokinetics of the drug eluting balloon systems with anticoagulant of Example 9 were evaluated in porcine coronary/intemal thoracic arteries in the non-diseased porcine coronary artery model. The balloon (e.g., the balloon of a balloon-catheter of a stent-delivery system) was advanced to a site to be treated (e.g., an occluded or weakened section of a blood vessel to be opened up or supported by a stent) in the body of a subject and inflated to a desired inflation diameter. Before, during, and/or after inflation of the balloon, the balloon released the therapeutic composition to, into, or at the treatment site, or to, into, or at an area adjacent thereto, by any suitable mechanism (e.g., concentration gradient, diffusion, pressure, or mechanical force, or a combination thereof). Safety of the device was evaluated at the 7- and 28-day time points following treatment in the coronary arteries and the tissue pharmacokinetics were evaluated following treatment in the coronary/thoracic arteries at the 7 and 28 day time points. Following treatment with the 14 mm length test drug coated balloon or the control plain balloon, an 18 mm length drug eluting balloon was deployed over the balloon treated segment of the coronary/thoracic artery. The tissue concentration is shown in ng/mg tissue.

The coated balloons were evaluated for drug delivery efficiency in an animal study. Drug transfer of the coated balloons into arterial segments were evaluated using harvested pig arteries. The arterial wall was separated after animal study. The arterial walls were then stored in an individual labeled vial. All samples were kept on dry ice until stored in the -80° C. freezer. All samples were then frozen to 70° C. prior to being analyzed. The tissue was extracted with Acetonitrile/methanol for Rivaroxaban, Apixaban, Novolimus and Rapamycin. The tissue content of Rivaroxaban, Apixaban, Novolimus and Rapamycin from the different drug coated balloon were analyzed using liquid chromatography mass spectroscopy (LCMS) with corresponding reference standards.

TABLE 7

Coated Balloon tissue concentration in vivo by different time period

| | | | | | |
|---|---|---|---|---|---|
| Balloon type | Balloon Coating information | Artery type | Drug | Time period | Tissue concentration (ng/mg) |
| SV300 18 mm Balloon | 300 µg Rivaroxaban in 150 µg Ethylene vinyl acetate and 150 µg Polyvinylpyrrolidone (MW=1.3 M) matrix | Coronary Artery | Rivaroxaban | 15 min | 1.23±0.37 (n=4) |
| | | Superficial Femoral Artery (SFA) | Rivaroxaban | 15 min | 40.58±64.71 (n=4) |
| VGR 14 mm Balloon | 800 µg Apixaban in 400 µg Polyethylene oxide (MW=8 M), 400 µg Polyvinylpyrrolidone (MW=1.3 M) and 4 µg Butylated hydroxytoluene matrix | Coronary Artery | Apixaban | Acute | 60.09±80.49 (n=2) |
| | | | | 1D | 0.08±0.08 (n=3) |
| | | | | 7D | 0.0001±0.0001 (n=3) |
| | | | | 28D | 0.003±0.005 (n=3) |
| NEV250 18 mm Balloon | 250 µg Novolimus in 125 µg Ethylene vinyl acetate and 125 µg Polyvinylpyrrolidone (MW=1.3 M) matrix | Coronary Artery | Novolimus | 6H | 6.52±3.68 (n=3) |
| | | | | 3D | 3.77±1.62 (n=3) |
| | | | | 7D | 1.47±0.37 (n=3) |
| | | Superficial Femoral Artery (SFA) | Novolimus | 6H | 5.94±3.66 (n=4) |
| | | | | 3D | 4.4±1.95(n=4) |
| | | | | 7D | 1.16±0.23 |
| NEV200 14 mm Balloon | 200 µg Novolimus in 100 µg Ethylene vinyl acetate and 100 µg Polyvinylpyrrolidone (MW=1.3 M) matrix | Coronary Artery | Novolimus | 7D | 0.60±0.64(n=7) |
| REV200 14 mm Balloon | 200 ug Rapamycin in 100 µg Ethylene vinyl acetate and 100 µg Polyvinylpyrrolidone (MW=1.3 M) matrix | Coronary Artery | Rapamycin | 7D | 1.45±1.23(n=8) |

As shown in Table 7, Rivaroxaban tissue concentration of tissue adjacent to the balloon treated segment ranges from at least 1.23 ng/mg within or at 15 min in coronary artery tissue to at least 40.58 ng/mg in Superficial Femoral Artery (SFA) tissue, within or at 15 minutes; Apixaban tissue concentration of tissue adjacent to the balloon treated segment in coronary artery tissue ranges from at least 60.09 ng/mg at acute to at least 0.08 ng/mg within or at 1 day to at least 0.0001 ng/mg within or at 7 days to at least 0.003 ng/mg within or at 28 days; Novolimus tissue concentration of tissue adjacent to the balloon treated segment in coronary artery tissue ranges from at least 6.52 ng/mg within or at 6 hours to at least 3.77 ng/mg within or at 3 day to at least 1.47 ng/mg within or at 7 days; Novolimus tissue concentration of tissue adjacent to the balloon treated segment in Superficial Femoral Artery (SFA)tissue ranges from at least 5.94 ng/mg within or at 6 hours to at least 4.4 ng/mg within or at 3 day to at least 1. 16 ng/mg within or at 7 days; Rapamycin tissue concentration of tissue adjacent to the balloon treated segment in coronary artery tissue ranges from at least 1.45 ng/mg within or at 7 days. Rivaroxaban, Apixaban, Novolimus or Rapamycin released locally sufficiently from a coated balloon catheter to inhibit smooth muscle proliferation after vessel injury at sufficient tissue concentration up to 7 days.

### Example 11: Preparation of Anticoagulant Eluting Valve Implant or Part of the Implant Not Covered by a Sleeve with Carrier

A valve or valve repair implant or part of the implant comprising the valve is coated with a coating containing anticoagulant Apixaban or Rivaroxaban and Argatroban..

Poly (L- lactide acid-co- glycolic acid) polymer is dissolved into dichloromethane at room temperature and vortex until the polymer had uniformly dissolved/dispersed. Anticoagulant (Apixaban or Rivaroxaban & Argatroban) are placed in a vial and dissolved in dichloromethane/Methanol at room temperature and vortex until all the drug was uniformly dissolved/dispersed.

Each polymer solution and each drug solutions are combined together (anticoagulant (Apixaban or Rivaroxaban & Argatroban with weight ratio 1 to 1) to poly (L-lactide acid-co- glycolic acid) by weight ratio was 3:1) according to the target drug dose.

The valve, and/or valve repair implant, and/or at least part of the implant comprising the valve optionally undergo surface treatment if the surface is not porous (i.e. plasma treatment or other surface friction treatment).

A microprocessor controlled ultrasonic sprayer was used to coat the valve of the drug containing carrier solution to the entire surface of the implant or part of the surface. After coating, the implant is placed in a 70° C. oven for about 2 hours to remove the residue solvent. The transcatheter valve or valve repair implant is then mounted on the delivery catheter. The catheters is then inserted in coils and packaged. The pouches were sterilized.

### Example 12: Preparation of Anticoagulant Eluting Valve Implant or Part of the Implant Covered by a Sleeve with Carrier

A valve or valve repair implant or part of the implant covered by a sleeve can have a polymer coating containing anticoagulant Apixaban or Rivaroxaban, Argatroban or a combination of both on top, part of, or adjacent to the sleeve made from ePTFE, Dacron, knitted or weaved fabric, or those known in the art.

The sleeve is infused with a polymer coating in a solvent solution with anticoagulant Apixaban or Rivaroxaban, Argatroban or a combination of both drugs and drug solution into the said sleeve and said solvent is allowed to evaporate leaving either polymer coating or drug in the pores of the sleeve. The sleeve is placed prior to being attached to the valve or valve repair implant or after it has been attached to the implant.

Poly (L- lactide acid-co- glycolic acid) polymer is dissolved into dichloromethane at room temperature and vortex until the polymer is uniformly dissolved/dispersed. Anticoagulant (Apixaban or Rivaroxaban & Argatroban) is placed in a vial and dissolved in dichloromethane/Methanol at room temperature and vortex until all the drug is uniformly dissolved/dispersed.

Each polymer solution and each drug solutions is combined together (anticoagulant (Apixaban or Rivaroxaban and Argatroban with weight ratio 1 to 1) to poly (L- lactide acid-co-glycolic acid) by weight ratio was 3:1) according to the target drug dose.

The sleeve optionally undergoes surface treatment if the surface is not porous (i.e. plasma treatment or other friction surface treatment). After surface treatment, the coating is spray coated or dip coated. The coating can be inside the sleeve or dipped onto the sleeve or coated on the sleeve.

When spray coated, a microprocessor controlled ultrasonic sprayer is used to coat the sleeve containing drug/excipient solution to the entire surface of the implant. After coating, the sleeve is placed in a 70° C. oven for about 2 hours to remove the residue solvent. The sleeve is then attached to the valve or valve repair implant if not prior to being attached to the implant. The valve or valve repair implant with sleeve attached is then mounted on the delivery catheter. The transcatheter valve or valve repair device is then inserted in a coil and packaged. The pouches were sterilized.

### Example 13: Preparation of Drug Eluting Stent Having Anticoagulant Argatroban Crosslinked with Poly N-(2-Hydroxyoropyl) Methacrylamide by Ester Linker

Example 13 includes methods for applying chemically crosslinked polymers and anticoagulant onto stent. The reactive polymer and anti-coagulant can be reacted and purified before making the coating solution or can be mixed together with initiator, then coated one layer for slow release of anticoagulant.

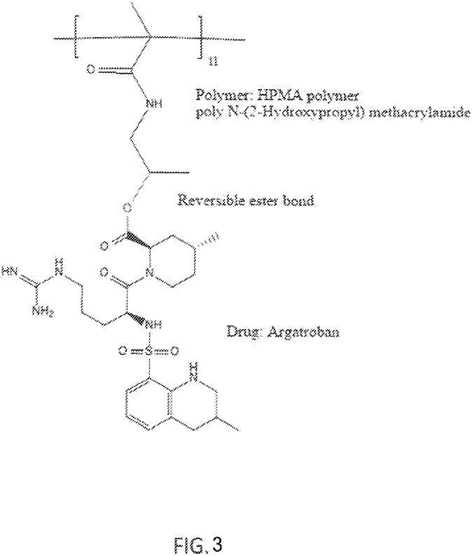

Anticoagulant (Apixaban or Rivaroxaban, Argatroban, Rivaroxaban etc.) is conjugated with biocompatible polymers via a reversible covalent bond, which can slowly release anticoagulant in a controlled manner. For example, Argatroban is linked to poly N-(2-Hydroxypropyl) methacrylamide by a reversible ester bond as show in FIG. 3. When this ester bond is hydrolyzed, the drug Argatroban is released.

Argatroban reacted with poly N-(2-Hydroxypropyl) methacrylamide is dissolved in THF. This polymer solution is air sprayed onto a stainless-steel coronary stent by a microprocessor controlled ultrasonic sprayer until a target weight achieved. After coating, the stents is placed in a 70° C. oven for about 2 hours to remove the solvent. The stents are then mounted on balloon catheters and crimped. The catheters are then inserted in coils and packaged. The pouches are sterilized.

In Vivo, when Argatroban - poly N-(2-Hydroxypropyl) methacrylamide ester bond is hydrolyzed, the drug Argatroban will be released.

### Example 14: Preparation of Drug Eluting Stent Having Anticoagulant Argatroban Crosslinked with PAMAM-OH Dendrimer by Ester Linker

PAMAM-OH dendrimer (generation 2) and Argatroban (1 to 1.2 mole ratio) are dissolved in THF, 4-Dimethylaminopyridine (DMAP) 1% was added as catalyst. The reaction mixture stirred overnight followed by purification. The purified Argatroban - PAMAM-OH dendrimer is dissolved in Tetrahydrofuran (THF). This polymer solution is air sprayed onto a stainless-steel coronary stent by a microprocessor controlled ultrasonic sprayer until a target weight achieved. After coating, the stents are placed in a 70° C. oven for about 2 hours to remove the solvent. The stents are then mounted on balloon catheters and crimped. The catheters are then inserted in coils and packaged. The pouches were sterilized.

In Vivo, when Argatroban crosslinked with PAMAM-OH dendrimer ester bond is hydrolyzed, the drug Argatroban will be released.

### Example 15: Preparation of Drug Eluting Stent Having Anticoagulant and Polymer Microsphere

Anticoagulant (Apixaban, Argatroban, Rivaroxaban etc.) are embodied within biocompatible materials (such as polymers, metals, ceramics, natural plant and/or animal materials). The polymers can be selected from polyesters (poly lactic acid, poly glycolic acid, Polyurethanes), Poly methyl methacrylate (PMMA), poly N-(2-Hydroxypropyl) methacrylamide, Polyethylenimine (PEI), dextran, dextrin, chitosans, poly(L-lysine), and poly(aspartamides), polyethylene, polypropylene, polyamides, Polyethylene glycol (PEG), Polytetrafluoroethylene (PTFE), Silicones, poly(anhydride), poly ortho esters etc. Anticoagulant (Apixaban, Argatroban, Rivaroxaban etc.) with polymers to form drug-polymer nano particles, microsphere, polymeric micelles as the polymer drug delivery systems, which can have high drug loading capacity in the hydrophobic core especially for hydrophobic drugs, and rapid cellular uptake facilitated by their nano-size characteristics.

0.5 mL of poly(D,L-lactide) dichloromethane solution (0.5% w/v) and anticoagulant( Rivaroxaban, Apixaban or Argatroban) dichloromethane solution (0.5% w/v) are slowly added dropwise to polyvinyl alcohol water solution(5% w/w) with magnetic stirring at 1000-1500 rpm. These dispersions are continue stirred for 2 hours at 40° C. and 200 rpm until the microspheres are very small (ie less than 1 µm in diameter) to form colloids and therefore the suspension does not settle under gravity. This polymer-anticoagulant suspension is dip coated multiple times to the stent surface until target drug weight achieved. The stent is air dried first then the stents are placed in a 70° C. oven for about 4 hours to remove the solvent. The stents are then mounted on balloon catheters and crimped. The catheters are then inserted in coils and packaged. The pouches were sterilized.

### Example 16: Preparation and Use of Anticoagulant-Impregnated Balloon

A balloon made of a biodegradable or non-degradable polymeric material (e.g., a nylon) and having openings (e.g., pores, holes, etc.) in the body and/or at the surface of the balloon is immersed in a mixture containing a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator, e.g. tPA) and a solvent, and optionally another kind of bioactive agent (e.g., an anti-proliferative agent, such as rapamycin or a derivative thereof, or an anti-inflammatory agent). The balloon can optionally have one or more coatings comprising a fibrin/thrombus formation-inhibiting or fibrin/thrombus dissolution-promoting agent and/or another kind of bioactive agent and can optionally have a coating comprising no bioactive agent.

The balloon (e.g., the balloon of a balloon-catheter of a stent-delivery system) is advanced to a site to be treated (e.g., an occluded or weakened section of a blood vessel to be opened up or supported by a stent) in the body of a subject and is inflated to a desired inflation diameter. Before, during and/or after inflation of the balloon, the balloon releases the fibrin/thrombus formation-inhibiting or fibrin/thrombus dissolution-promoting agent and the optional other kind of bioactive agent (e.g., an anti-proliferative agent or an anti-inflammatory agent) to, into or at the treatment site, or to, into or at an area adjacent thereto, by any suitable mechanism (e.g., concentration gradient, diffusion, pressure or mechanical force, or a combination thereof).

### Example 17: Preparation and Use of Anticoasulant-Coated Catheter

A first mixture containing a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator, e.g. tPA) and a solvent, and optionally a biodegradable or non-degradable polymeric material and/or another kind of bioactive agent (e.g., an anti-proliferative agent, such as rapamycin or a derivative thereof, or an anti-inflammatory agent), is applied (e.g., by spraying or dipping) to a catheter made of a biodegradable or non-degradable polymeric material (e.g., a nylon or a polyether block amide, such as PEBAX®) to form a first coating on the catheter. The first coating can cover any surfaces (e.g., the exterior surface, any other surfaces or all surfaces) of the catheter.

Optionally, a second mixture containing another kind of bioactive agent (e.g., an anti-proliferative agent, such as rapamycin or a derivative thereof, or an anti-inflammatory agent) and a solvent, and optionally a biodegradable or non-degradable polymeric material and/or a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator, e.g. tPA), is applied to the catheter (e.g., by spraying or dipping) to form a second coating on the catheter. The optional second coating can cover any surfaces (e.g., the exterior surface, any other surfaces or all surfaces) of the catheter. The first mixture and the optional second mixture can be applied to the catheter in any order.

Optionally, a third mixture containing a biodegradable or non-degradable polymeric material and a solvent is applied to the catheter (e.g., by spraying or dipping) to form a third coating over the first coating and the optional second coating. The optional third coating can be, e.g., a top layer or coat or a diffusion barrier that controls release of the fibrin/thrombus formation-inhibiting or fibrin/thrombus dissolution-promoting agent and the optional other kind of bioactive agent from the first coating and the optional second coating. The optional third coating can cover the exterior surface or any other surfaces, or all surfaces, of the catheter.

The coated catheter is optionally heated to stabilize the coating(s) and is placed in a container (e.g., a pouch) and sterilized (e.g., by exposure to e-beam radiation).

The catheter (e.g., the catheter of a stent-delivery system) is advanced to a site to be treated (e.g., an occluded or weakened section of a blood vessel to be opened up or supported by a stent) in the body of a subject. Before, while and/or after the catheter is positioned at the treatment site or at an area adjacent thereto, the catheter releases the fibrin/thrombus formation-inhibiting or fibrin/thrombus dissolution-promoting agent and the optional other kind of bioactive agent (e.g., an anti-proliferative agent or an anti-inflammatory agent) to, into or at the treatment site, or to, into or at an area adjacent thereto, by any suitable mechanism (e.g., concentration gradient, diffusion, pressure or mechanical force, or a combination thereof).

### Example 18: Preparation and Use of Anticoagulant-Impregnated Catheter

A catheter (e.g., an infusion catheter) made of a biodegradable or non-degradable polymeric material (e.g., a nylon or a polyether block amide, such as PEBAX®) and having openings (e.g., pores, holes, etc.) in the body and/or at the surface of the catheter is immersed in a mixture containing a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator) and a solvent, and optionally another kind of bioactive agent (e.g., an anti-proliferative agent, such as rapamycin or a derivative thereof, or an anti-inflammatory agent). The catheter can optionally have one or more coatings comprising a fibrin/thrombus formation-inhibiting or fibrin/thrombus dissolution-promoting agent and/or another kind of bioactive agent and can optionally have a coating comprising no bioactive agent.

The catheter (e.g., the catheter of a stent-delivery system) is advanced to a site to be treated (e.g., an occluded or weakened section of a blood vessel to be opened up or supported by a stent) in the body of a subject. Before, while and/or after the catheter is positioned at the treatment site or at an area adjacent thereto, the catheter releases the fibrin/thrombus formation-inhibiting or fibrin/thrombus dissolution-promoting agent and the optional other kind of bioactive agent (e.g., an anti-proliferative agent or an anti-inflammatory agent) to, into or at the treatment site, or to, into or at an area adjacent thereto, by any suitable mechanism (e.g., concentration gradient, diffusion, pressure or mechanical force, or a combination thereof).

### Example 19: Preparation and Use of Anticoagulant-Coated Balloon-Catheter

A first mixture containing a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator) and a solvent, and optionally a biodegradable or non-degradable polymeric material and/or another kind of bioactive agent (e.g., an anti-proliferative agent, such as rapamycin or a derivative thereof, or an anti-inflammatory agent), is applied (e.g., by spraying or dipping) to the balloon portion and/or the catheter portion of a balloon-catheter made of a biodegradable or non-degradable polymeric material (e.g., a nylon) to form a first coating on the balloon portion and/or the catheter portion. The first coating can cover any surfaces (e.g., the exterior surface, any other surfaces or all surfaces) of the balloon and/or the catheter.

Optionally, a second mixture containing another type of bioactive agent (e.g., an anti-proliferative agent, such as rapamycin or a derivative thereof, or an anti-inflammatory agent) and a solvent, and optionally a biodegradable or non-degradable polymeric material and/or a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator), is applied to the balloon portion and/or the catheter portion (e.g., by spraying or dipping) to form a second coating on the balloon portion and/or the catheter portion. The optional second coating can cover any surfaces (e.g., the exterior surface, any other surfaces or all surfaces) of the balloon and/or the catheter. The first mixture and the optional second mixture can be applied to the balloon portion and/or the catheter portion in any order.

Optionally, a third mixture containing a biodegradable or non-degradable polymeric material and a solvent is applied to the balloon portion and/or the catheter portion (e.g., by spraying or dipping) to form a third coating over the first coating and the optional second coating. The optional third coating can be, e.g., a top layer or coat or a diffusion barrier that controls release of the fibrin/thrombus formation-inhibiting or fibrin/thrombus dissolution-promoting agent and the optional other kind of bioactive agent from the first coating and the optional second coating. The optional third coating can cover the exterior surface or any other surfaces, or all surfaces, of the balloon and/or the catheter.

The coated balloon-catheter is optionally heated to stabilize the coating(s) and is placed in a container (e.g., a pouch) and sterilized (e.g., by exposure to e-beam radiation).

The balloon-catheter (e.g., the balloon-catheter of a stent-delivery system) is advanced to a site to be treated (e.g., an occluded or weakened section of a blood vessel to be opened up or supported by a stent) in the body of a subject, and the balloon is inflated to a desired inflation diameter. Before, during and/or after inflation of the balloon, the balloon portion and/or the catheter portion of the balloon-catheter releases the fibrin/thrombus formation-inhibiting or fibrin/thrombus dissolution-promoting agent and the optional other kind of bioactive agent (e.g., an anti-proliferative agent or an anti-inflammatory agent) to, into or at the treatment site, or to, into or at an area adjacent thereto, by any suitable mechanism (e.g., concentration gradient, diffusion, pressure or mechanical force, or a combination thereof). Example 20: Preparation and use of anticoagulant-impregnated balloon-catheter

The balloon portion and/or the catheter portion of a balloon-catheter (e.g., a weeping catheter) made of a biodegradable or non-degradable polymeric material (e.g., a nylon) and having openings (e.g., pores, holes, etc.) in the body and/or at the surface of the balloon and/or the catheter is immersed in a mixture containing a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator) and a solvent, and optionally another type of bioactive agent (e.g., an anti-proliferative agent, such as rapamycin or a derivative thereof, or an anti-inflammatory agent). The balloon portion and/or the catheter portion of the balloon-catheter can optionally have one or more coatings comprising a fibrin/thrombus formation-inhibiting or fibrin/thrombus dissolution-promoting agent and/or another kind of bioactive agent, and can optionally have a coating comprising no bioactive agent.

The balloon-catheter (e.g., the balloon-catheter of a stent-delivery system) is advanced to a site to be treated (e.g., an occluded or weakened section of a blood vessel to be opened up or supported by a stent) in the body of a subject, and the balloon is inflated to a desired inflation diameter. Before, during and/or after inflation of the balloon, the balloon portion and/or the catheter portion of the balloon-catheter releases the fibrin/thrombus formation-inhibiting or fibrin/thrombus dissolution-promoting agent and the optional other kind of bioactive agent (e.g., an anti-proliferative agent or an anti-inflammatory agent) to, into or at the treatment site, or to, into or at an area adjacent thereto, by any suitable mechanism (e.g., concentration gradient, diffusion, pressure or mechanical force, or a combination thereof).

### Example 21: Use of Anticoagulant-Delivering Infusion Catheter

An infusion catheter contains one or more lumens for delivering one or more drugs. The infusion catheter (e.g., the catheter of a stent-delivery system) is advanced to a site to be treated (e.g., an occluded or weakened section of a blood vessel to be opened up or supported by a stent) in the body of a subject. Before, while and/or after the catheter is positioned at the treatment site or at an area adjacent thereto, a first mixture containing a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator) and a solvent (e.g., saline), and optionally another kind of bioactive agent (e.g., an anti-proliferative agent, such as rapamycin or a derivative thereof, or an anti-inflammatory agent), is injected through one or more drug-delivering lumens of the catheter. Before, while and/or after the catheter is positioned at the treatment site or at an area adjacent thereto, a second mixture containing another kind of bioactive agent (e.g., an anti-proliferative agent or an anti-inflammatory agent) and a solvent (e.g., saline), and optionally a fibrin/thrombus formation-inhibiting or fibrin/thrombus dissolution-promoting agent, optionally is injected through one or more drug-delivering lumens of the catheter. The catheter delivers the fibrin/thrombus formation-inhibiting or fibrin/thrombus dissolution-promoting agent and the optional other kind of bioactive agent (e.g., an anti-proliferative agent or an anti-inflammatory agent) to, into or at the treatment site, or to, into or at an area adjacent thereto.

### Example 22: Preparation and Use of Anticoagulant-Coated Surgical Instrument

A surgical instrument (e.g., a cutting instrument, such as a knife) made of a biodegradable or non-degradable metal or metal alloy (e.g., stainless steel) optionally undergoes surface treatment (e.g., microblasting) to improve adhesion of a coating (e.g., a polymeric coating) to a metal surface.

A first mixture containing a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator) and a solvent, and optionally a biodegradable or non-degradable polymeric material and/or another kind of bioactive agent (e.g., an anti-proliferative agent, such as rapamycin or a derivative thereof, or an anti-inflammatory agent), is applied to the surgical instrument (e.g., by dipping or spraying) to form a first coating on the surgical instrument. The first coating can cover the exterior surface, any other surfaces or all surfaces of the surgical instrument.

Optionally, a second mixture containing another kind of bioactive agent (e.g., an anti-proliferative agent, such as rapamycin or a derivative thereof, or an anti-inflammatory agent) and a solvent, and optionally a biodegradable or non-degradable polymeric material and/or a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator), is applied to the surgical instrument (e.g., by dipping or spraying) to form a second coating on the surgical instrument. The optional second coating can cover the exterior surface, any other surfaces or all surfaces of the surgical instrument. The first mixture and the optional second mixture can be applied to the surgical instrument in any order.

Optionally, a third mixture containing a biodegradable or non-degradable polymeric material and a solvent is applied to the surgical instrument (e.g., by dipping or spraying) to form a third coating over the first coating and the optional second coating. The optional third coating can be, e.g., a top layer or coat or a diffusion barrier that controls release of the fibrin/thrombus formation-inhibiting or fibrin/thrombus dissolution-promoting agent and the optional other kind of bioactive agent from the first coating and the optional second coating. The optional third coating can cover the exterior surface, any other surfaces or all surfaces of the surgical instrument.

The coated surgical instrument is optionally heated to stabilize the coating(s) and is placed in a container (e.g., a pouch) and sterilized (e.g., by exposure to e-beam radiation).

The surgical instrument (e.g., a cutting instrument, such as a knife) is advanced to a site in the body of a subject undergoing a surgery or intervention (e.g., a tissue to be cut or treated). Before, while and/or after the surgical instrument is positioned at the treatment site or at an area adjacent thereto, or before, while and/or after the surgical instrument contacts the tissue to be treated, the surgical instrument releases the fibrin/thrombus formation-inhibiting or fibrin/thrombus dissolution-promoting agent and the optional other kind of bioactive agent (e.g., an anti-proliferative agent or an anti-inflammatory agent) to, into or at the treatment site, or to, into or at an area adjacent thereto, by any suitable mechanism (e.g., concentration gradient, diffusion, pressure or mechanical force, or a combination thereof).

### Example 23: Preparation and Use of Anticoagulant-Impregnated Surgical Instrument

A surgical instrument made of a biodegradable or non-degradable metal or metal alloy (e.g., stainless steel) and having openings (e.g., pores, holes, etc.) in the body and/or at the surface of the surgical instrument is immersed in a mixture containing a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator) and a solvent, and optionally another kind of bioactive agent (e.g., an anti-proliferative agent, such as rapamycin or a derivative thereof, or an anti-inflammatory agent). The surgical instrument can optionally undergo surface treatment, can optionally have one or more coatings comprising a fibrin/thrombus formation-inhibiting or fibrin/thrombus dissolution-promoting agent and/or another kind of bioactive agent, and can optionally have a coating comprising no bioactive agent.

The surgical instrument (e.g., a cutting instrument, such as a knife) is advanced to a site in the body of a subject undergoing a surgery or intervention (e.g., a tissue to be cut or treated). Before, while and/or after the surgical instrument is positioned at the treatment site or at an area adjacent thereto, or before, while and/or after the surgical instrument contacts the tissue to be treated, the surgical instrument releases the fibrin/thrombus formation-inhibiting or fibrin/thrombus dissolution-promoting agent and the optional other kind of bioactive agent (e.g., an anti-proliferative agent or an anti-inflammatory agent) to, into or at the treatment site, or to, into or at an area adjacent thereto, by any suitable mechanism (e.g., concentration gradient, diffusion, pressure or mechanical force, or a combination thereof).

### Example 24: Use of Anticoagulant-Delivering Infusion Surgical Instrument

An infusion surgical instrument [e.g., a cutting instrument (e.g., a knife) or an injection device (e.g., a needle)] contains one or more lumens for delivering one or more drugs. The surgical instrument is advanced to a site in or on the body of a subject undergoing a surgery or intervention (e.g., a tissue to be cut or treated). Before, while and/or after the surgical instrument is positioned at the treatment site or at an area adjacent thereto, or before, while and/or after the surgical instrument contacts the tissue to be treated, a first mixture containing a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator) and a solvent (e.g., saline), and optionally another kind of bioactive agent (e.g., an anti-proliferative agent, such as rapamycin or a derivative thereof, or an anti-inflammatory agent), is injected through one or more drug-delivering lumens of the surgical instrument. Before, while and/or after the surgical instrument is positioned at the treatment site or at an area adjacent thereto, or before, while and/or after the surgical instrument contacts the tissue to be treated, a second mixture containing another kind of bioactive agent (e.g., an anti-proliferative agent or an anti-inflammatory agent) and a solvent (e.g., saline), and optionally a fibrin/thrombus formation-inhibiting or fibrin/thrombus dissolution-promoting agent, optionally is injected through one or more drug-delivering lumens of the surgical instrument. The surgical instrument delivers the fibrin/thrombus formation-inhibiting or fibrin/thrombus dissolution-promoting agent and the optional other kind of bioactive agent (e.g., an anti-proliferative agent or an anti-inflammatory agent) to, into or at the treatment site, or to, into or at an area adjacent thereto.

### Example 25 Preparation of mTOR Inhibitors and/or Anticoagulant Coated Balloon with Excipient

The balloon can optionally adopt carrier excipient to coat to facilitate drug transfer to the vessel wall and control release rate. A variety of carrier excipients and techniques can be used. The selected excipient could be contrast agent (i.e. iopromide), urea, dextrane, shellac, shelloic acid, keratosis (a naturally derived protein), Plasticizer (i.e. butyryl-tri-hexyl citrate,acetyl tributyl citrate, citrate ester, glycerol, other organic ester), hydrophilic space, Polyvinylpyrrolidone (PVP) and its hydrogels, Surfactants, Non-ionic surfactant Polysorbate/sorbitol (i.e. Tween20, Tween60 or Tween80), nordihydroguaiaretic acid (NDGA), hydrophibic excipient such as phospholipid, amphiphilic polymer such as Poly(ethylene glycol) (i.e PEG 8000), poly(ethylene oxide) (PEO) (molecular weight range from 100,000 to 10,000,000), Polyethylenimine (PEI) or polyaziridine linear or branched, amphiphilic block co-polymers composed of poly(ethylene oxide) (PEO) as the hydrophilic block and poly(ether)s, poly(amino acid)s), hydrophobic polymer space, biodegradable polymers such as Poly DL lactide-co-glycolide, Poly L Lactide-co-caprolactone, durable polymers, individually or combinations thereof.

A balloon made of a biodegradable or non-degradable polymeric material (e.g., a nylon) and having openings (e.g., pores, holes, etc.) in the body and/or at the surface of the balloon was used. The drug was selected from a mixture containing a mTOR inhibitors /Anticoagulant1/ Anticoagulant2. The mTOR inhibitors was selected from Sirolimus, Novolimus, temsirolimus, zotarolimus and everolimus etc. The anticoagulants were selected from Apixaban, Argatroban, Rivaroxaban or a derivative thereof, and/or a fibrinolytic or thrombolytic agent, such as a plasminogen activator) individually or combinations thereof. For example, Siroliums and Anticoagulant1/ Anticoagulant2 (Apixaban and Argatroban) were placed in a vial and dissolved in dichloromethane or dichloromethane/Methanol combination at 2 to 10 mg/ml. The carrier excipient was dissolved in a proper solvent. The solution and drug solutions were combined at a target ratio of 3 to 1, 1 to 1, 2 to 1, or 1 to 3 ratios according to the target drug loading. Further dilution with dichloromethane was conducted if needed. Optionally anti-solvent was used to control the coating morphology of particles and drug release rate.

Optionally the balloon can undergo physical surface treatment before coated such that the surface has microspores, micro-holes, or chemical surface treatment before coated such that the balloon materials have photo-link or other chemical function group that can be reacted under UV or other techniques to easy coating. After surface treatment, the coating can be spray coat or dip coat or use other coating techniques (i.e. 3D printer).

The coating can optionally cover any surfaces (e.g., the exterior surface, any other surfaces or all surfaces) of the balloon. The coating solution can be homogeneous or non-homogeneous such as suspension or emulsions.

The coated balloon can optionally combine multi-strategies (e.g., electrospinning, plasma treatment, Layer-by-Layer Self-Assembly or a combination thereof) to form finely controlling structural, mechanical, and surface properties. With tuned coating techniques and solvent removal technique, the produces powder particles can be optionally homogenous, porous, and uniform in size and shape. The morphology of particles could be micro-crystalline, nanoparticles, Nano-encapsulated to provide release rate control.

When spray coat, a microprocessor controlled ultrasonic sprayer was used to apply the drug containing drug solution to cover any surface of a balloon. A mandrel was placed through catheter tips and underneath an ultrasonic spray nozzle (Micromist System with Ultrasonic Atomizing Nozzle Sprayer, Sono-Tek, N.Y.), which was rotating at 80 rpm and move longitudinally at a rate of 0.050 inches/minutes. The coating parameter can optionally adjusted to ideal coating texture and the morphology and the profile of the interface between drug and balloon surface.

After coating, the balloon was placed in a vacuum chamber to remove the residue solvent. Optionally the coated balloon can be tri-folded to protect coated drug with a folded and/or wrapped balloon thereon to a pre-annealing step to induce a fold/wrap memory in the resulting pre-annealed balloon and/or coated balloon has a protector which need to peel off before use.

The balloon catheter was then inserted in a coil and packaged. The pouch was sterilized by Ethylene oxide or E-beam. The pouch was further packaged in a foil pouch with oxygen scavengers and nitrogen purge and vacuum sealed.

The balloon (e.g., the balloon of a balloon-catheter of a balloon-delivery system) was advanced to a site to be treated (e.g., an occluded or weakened section of a blood vessel to be opened up or supported by a balloon) in the body of a subject and was inflated to a desired inflation diameter. Before, during and/or after inflation of the balloon, the balloon releases the fibrin/thrombus formation-inhibiting or fibrin/thrombus dissolution-promoting agent and the optional other kind of bioactive agent (e.g., an anti-proliferative agent or an anti-inflammatory agent) to, into or at the treatment site, or to, into or at an area adjacent thereto, by any suitable mechanism (e.g., concentration gradient, diffusion, pressure or mechanical force, or a combination thereof).

### Example 26: Preparation of Drug Coated Balloon Having Anticoagulant and Polymer Microsphere

Anticoagulant (Apixaban, Argatroban, Rivaroxaban etc.) are embodied within biocompatible materials (such as polymers, metals, ceramics, albumin, liposome, natural plant and/or animal materials). The polymers can be selected from polyesters (poly lactic acid, poly glycolic acid, poly lactic acid-co- glycolic acid, poly lactic acid-co-caprolactone, poly ethylene glycol-block- poly caprolactone, Polyurethanes etc.), Poly methyl methacrylate (PMMA), poly N-(2-Hydroxypropyl) methacrylamide, Polyethylenimine (PEI), dextran, dextrin, chitosans, poly(L-lysine), and poly(aspartamides), polyethylene, polypropylene, polyamides, Polyethylene glycol (PEG), Silicones, poly(anhydride), poly ortho esters etc. Anticoagulant (Apixaban, Argatroban, Rivaroxaban etc.) with polymers to form drug-polymer nano particles, microsphere, polymeric micelles as the polymer drug delivery systems, which can have high drug loading capacity in the hydrophobic core especially for hydrophobic drugs, and rapid cellular uptake facilitated by their micro/nano-size characteristics.

0.5 mL of poly(D,L-lactide) dichloromethane solution (0.5% w/v) and anticoagulant( Rivaroxaban, Apixaban or Argatroban) dichloromethane solution (0.5% w / v) are slowly added dropwise to polyvinyl alcohol water solution(5% w/w) with magnetic stirring at 1000-1500 rpm. These dispersions are continue stirred for 2 hours at 40° C. and 200 rpm until the microspheres are very small (ie less than 1 µm in diameter) to form colloids and therefore the suspension does not settle under gravity. This polymer-anticoagulant suspension is dip coated multiple times to the balloon surface until target drug weight achieved. After coating, the balloons were air dried first then were placed in a vacuum chamber to remove the solvent. The balloons were then tri-folded before putting on the protective sheath. The balloon catheters were then inserted in coils and packaged. The pouches were sterilized.

### Example 27: Preparation of Drug Coated Balloon Having Anticoagulant and Polymer Self-Assembly Hollow Nanoparticles

Anticoagulant (Apixaban, Argatroban, Rivaroxaban etc.) are monodispersed within hollow polymer nanoparticle. The polymers with molecular range from 100 K to 10 K ( optimally from 40 k to 20 K) can be selected from block degradable polymers of poly lactic acid, poly glycolic acid, poly lactic acid-co- glycolic acid, poly lactic acid-co-caprolactone, poly ethylene glycol-block- poly caprolactone etc., or block non-degradable polymers selected from Polyvinylpyridine block with Poly methyl methacrylate (PMMA), poly N-(2-Hydroxypropyl) methacrylamide, Polyethylenimine (PEI), dextran, dextrin, chitosans, poly(L-lysine), and poly(aspartamides), polyamides, Polyethylene glycol (PEG), Silicones, poly(anhydride), poly ortho esters, polystyrene-b-polyvinylpyridine, poly(isoprene)-b-poly(vinyl pyridine), poly(vinyl pyridine)-b-poly(styrene)-b-poly(vinyl pyridine), poly(styrene)-b-polyvinyl pyridine)-b-poly(styrene), poly(styrene-b-poly(acrylic acid), poly(styrene)-b-poly(butadiene)-b-poly(vinyl pyridine), poly(styrene-b-poly(methacrylic acid), poly(styrene)-b-poly(ethylene oxide), poly(butadiene)-b-poly(acrylic acid), poly(butadiene)-b-poly(ethylene oxide), poly(vinyl pyridine)-b-poly(butadiene)-b-poly(vinyl pyridine), poly(ethylene)-b-poly(ethylene oxide), and poly(styrene)-b-poly(vinyl pyridine)-b-poly(ethylene oxide) etc. Anticoagulant (Apixaban, Argatroban, Rivaroxaban etc.) with polymers to form drug-polymer nano particles as the polymer drug delivery systems, which can have high drug loading capacity in the hydrophobic core especially for hydrophobic drugs, and rapid cellular uptake facilitated by their nano-size characteristics.

Poly(ethylene glycol)-block-poly(4-vinyl pyridine) or poly(styrene)-block-poly(4-vinyl pyridine) or other poly(4-vinyl pyridine) block polymers is dissolved in DMF or 1,4-dioxane to prepare a solution of 5 mg/ml; this solution is added to solutions containing varied amount of Azo compounds in the same solvent(the monomer molar ratio of 4-vinyl pyridine: Azo compounds from 1:0.2 to 1:2). Azo compounds are selected from Metanil Yellow, Orange II sodium salt,2,2′-Dihydroxyazobenzene,2-(4-Hydroxyphenylazo) benzoic acid,5-[(2-Carboxyphenyl) azo]-2-hydroxybenzoic acid, Olsalazine, 5-[(4-aminophenyl)azo]-2-hydroxy-Benzoic acid as hydrogen bonding agent for self-assembly. After stirring and reflux overnight, the self-assembly nanoparticles were collected by centrifuging. Using ethanol wash to remove hydrogen bonding agent results in monodisperse hollow nanoparticles with tunable hollow cavity size and internal surface reactivity. The resulting nanoparticles are redispersed in chloroform and mixed with anticoagulant (Rivaroxaban, Apixaban or Argatroban) solution in the same solvent; the balloon can be coated with this solution by dip- or spin-coating method to the balloon surface until target drug weight achieved with anticoagulant (Rivaroxaban, Apixaban or Argatroban) is hydrogen bonding with this hollow nanoparticle polymers.

After coating, the balloons were air dried first then were placed in a vacuum chamber to remove the solvent. The balloons were then tri-folded before putting on the protective sheath. The balloon catheters were then inserted in coils and packaged. The pouches were sterilized.

### Example 28: Preparation of Drug Coated Balloon Having Colocalized Synergized Delivery of m-TOR and Paclitaxel Self-Assembly Hollow Nanoparticles

m-TOR (Sirolimus, biolimus, everolimus, myolimus, novolimus, ridaforolimus, temsirolimus, zotarolimus, or salts, isomers, solvates, analogs, derivatives, metabolites etc.) and paclitaxel are embodied within hollow polymer nanoparticles. The polymers with molecular range from 100 K to 10 K ( optimally from 40 k to 20 K) can be selected from block degradable polymers of poly lactic acid, poly glycolic acid, poly lactic acid-co- glycolic acid, poly lactic acid-co-caprolactone, poly ethylene glycol-block- poly caprolactone etc., or block non-degradable polymers selected from Polyvinylpyridine block with Poly methyl methacrylate (PMMA), poly N-(2-Hydroxypropyl) methacrylamide, Polyethylenimine (PEI), dextran, dextrin, chitosans, poly(L-lysine), and poly(aspartamides), polyamides, Polyethylene glycol (PEG), Silicones, poly(anhydride), poly ortho esters, polystyrene-b-polyvinylpyridine, poly(isoprene)-b-poly(vinyl pyridine), poly(vinyl pyridine)-b-poly(styrene)-b-poly(vinyl pyridine), poly(styrene)-b-polyvinyl pyridine)-b-poly(styrene), poly(styrene-b-poly(acrylic acid), poly(styrene)-b-poly(butadiene)-b-poly(vinyl pyridine), poly(styrene-b-poly(methacrylic acid), poly(styrene)-b-poly(ethylene oxide), poly(butadiene)-b-poly(acrylic acid), poly(butadiene)-b-poly(ethylene oxide), poly(vinyl pyridine)-b-poly(butadiene)-b-poly(vinyl pyridine), poly(ethylene)-b-poly(ethylene oxide), and poly(styrene)-b-poly(vinyl pyridine)-b-poly(ethylene oxide). M-TOR with paclitaxel and polymers to form drug-polymer nano particles as the polymer drug delivery systems, which can have high drug loading capacity in the hydrophobic core especially for hydrophobic drugs, and rapid cellular uptake facilitated by their nano-size characteristics.

Poly(ethylene glycol)-block-poly(4-vinyl pyridine) or poly(styrene)-block-poly(4-vinyl pyridine) or other poly(4-vinyl pyridine) block polymers is dissolved in DMF or 1,4-dioxane to prepare a solution of 5 mg/ml; this solution is added to solutions containing varied amount of Azo compounds in the same solvent(the monomer molar ratio of 4-vinyl pyridine: Azo compounds from 1:0.2 to 1:2). Azo compounds are selected from Metanil Yellow, Orange II sodium salt, 2,2′-Dihydroxyazobenzene,2-(4-Hydroxyphenylazo) benzoic acid,5-[(2-Carboxyphenyl) azo]-2-hydroxybenzoic acid, Olsalazine, 5-[(4-aminophenyl) azo]-2-hydroxy-Benzoic acid as hydrogen bonding agent. After stirring and reflux overnight, the nanoparticles were collected by centrifuging. Using ethanol wash to remove hydrogen bonding agent results in self-assembly monodisperse hollow nanoparticles with tunable hollow cavity size and internal surface reactivity. The resulting nanoparticles are redispersed in chloroform and mixed with m-TOR and paclitaxel (ranging from 3:1 to 1:3 by weight) solution in the same solvent; the balloon can be coated with this solution by dip- or spin-coating method to the balloon surface until target drug weight achieved with m-TOR and paclitaxel are hydrogen bonding with this self-assembly hollow nanoparticle.

After coating, the balloons were air dried first then were placed in a vacuum chamber to remove the solvent. The balloons were then tri-folded before putting on the protective sheath. The balloon catheters were then inserted in coils and packaged. The pouches were sterilized.

### Example 29: Preparation of Anticoagulant and/or mTOR Coated Sleeve in a Covered Stent

A stent covered by a sleeve can have a polymer coating containing anticoagulant and/or a combination with mTOR on top, part of, and /or adjacent to the sleeve made from polymer selected from non-degradable polymers such as polytetrafluoroethylene, fluorinated ethylene propylene, Dacron, polyethylene terephthalate, polyurethanes, polycarbonate, polypropylene, Pebax, polyethylene and biological polymers such as modified cellulose, collagen, fibrin, and elastin, and biodegradable polymer such as poly(alpha-hydroxy acid), poly-L-lactide (PLLA), poly-D-lactide (PDLA), polyglycolide (PGA), polydioxanone, polycaprolactone (PCL), polygluconate, polylactic acid-polyethylene oxide copolymers, poly(hydroxybutyrate), polyanhydride, polyphosphoester, or poly(aminoacides), knitted or weaved fabric material or film material which have been previously cast by brushing, dipping, electrospun or electrospray technique or other means onto the metallic bare or polymer stent, or those known in the art, which coated with anticoagulant and/or mTOR. The sleeve surface can be porous or non-porous.

The sleeve can be infused with a polymer coating in a solvent solution with anticoagulant such as Apixaban, Rivaroxaban, Argatroban or a combination with mTOR such as rapamycin, everolimus, biolimus, temsirolimus, ridaforolimus, zotarolimus, myolimus and novolimus drug solution into the said sleeve and said solvent can evaporate leaving either polymer coating or drug in the pores of the sleeve. This process also applicable to stent graft.

Poly (L- lactide acid-co- glycolic acid) polymer is dissolved into dichloromethane at room temperature and vortex until the polymer is uniformly dissolved/dispersed. Anticoagulant (Apixaban or Rivaroxaban & Argatroban) is placed in a vial and dissolved in dichloromethane/Methanol at room temperature and vortex until all the drug is uniformly dissolved/dispersed.

Each polymer solution and each drug solutions is combined anticoagulant (Apixaban or Rivaroxaban) and Argatroban and/or mTOR with weight ratio 1 to 1 or other ratio) to poly (L- lactide acid-co- glycolic acid) by weight ratio was 3:1 or other ratio according to the target drug dose.

The sleeve can optionally undergo surface treatment if the surface is not porous (i.e. plasma treatment or other friction surface treatment). After surface treatment, the coating could be spray coated or dip coated. The coating can be inside the sleeve or dipped onto the sleeve or coated on the sleeve.

When spray coated, a microprocessor controlled ultrasonic sprayer is used to coat the sleeve containing drug/excipient solution to the entire surface of the implant. After coating, the sleeve is placed in a 70° C. oven for about 2 hours to remove the residue solvent. The covered stent is then mounted on the delivery catheter and then is inserted in a coil and packaged. The pouches were sterilized.

### Example 30: Preparation of m-TOR Organic Formulation With Self-Emulsifying Agent for Use in Eye Injection

Novolimus at concentration of about 200 mg/mL was dissolved in 95% PEG 300 NF with 5% Ethanol 190 Proof USP. Myolimus at concentration of about 175 mg/mL was dissolved in 95% PEG 300 NF with 5% Ethanol 190 Proof USP. These drug solutions were injected (10 to 23 µL) into rabbit and swine eye with drug concentrations in target eye tissues and whole blood as shown in Tables 7A, 7C, 8A, and 8C. Drug concentrations in the eye tissues were measured using High Performance Liquid Chromatography (HPLC). Drug concentrations in the blood were measured using HPLC-Mass Spectrometry (HPLC-MS). In addition, ocular pressure was measured during study as shown in Tables 7B and 8B. Ocular pressure was measured using a Model 30 ClassicT™ pneumatonometer.

TABLE 7A

Tissue Concentrations from Novolimus Formulation I Injection into Rabbit Eye and Swine Eye

| Animal Group | Formulation (Dose in mg) | Time Point (Days) | Average Amount of Drug per Tissue Mass (ng/g) | | | |
|---|---|---|---|---|---|---|
| | | | Vitreous | Retina | Choroid | Sclera |
| G rabbit | Formulation I (20 µl; 4 mg) | 1 | 25328 | 10948 | 3264 | 760 |
| C and D rabbits | Formulation I (20 µl; 4 mg) | 72 | 332 | 76 | 92 | 52 |
| A and B rabbits | Formulation I (10 µl; 2 mg) | 72 | 2 | 4 | 8 | 4 |
| E swine | Formulation I (20 µl; 4 mg) | 1 | 101621 | 135598 | 44736 | 4331 |

TABLE 7B

Intraocular Pressure of Rabbit Eye from Novolimus Formulation I Injection

| Animal Group | Formulation (Dose in mg) | Intraocular Pressure (mm Hg) | | | |
|---|---|---|---|---|---|
| | | Day 1 | | Later Time Point (Day) | |
| | | pre-treatment* | post-treatment* | | |
| G rabbit | Formulation I (20 µl; 4 mg) | 12.0 | 13.0 | Day 2 24.3 | |
| C and D rabbits | Formulation I (20 µl; 4 mg) | 17.0 (Control) 16.8 (Test Article) | 26.5 (Control) 29.5 (Test Article) | Day 8/10 29.3 (Control) 10.8 (Test Article) | Day 71 25.5 (Control) 26.5 (Test Article) |
| A and B rabbits | Formulation I (10 µl; 2 mg) | 21.5 (Control) 23.0 (Test Article) | 25.3 (Control) 24.0 (Test Article) | Day 8/10 27.3 (Control) 23.5 (Test Article) | Day 71 21.0 (Control) 24.3 (Test Article) |

* Under anesthetic and dilation

TABLE 7C

Whole Blood Concentration from Novolimus Formulation I Injection into Rabbit Eye

| Animal Group | Formulation (Dose in mg) | Time Point (Days) | Whole Blood Concentration (ng/mL) |
|---|---|---|---|
| G Rabbit | Formulation I (20 µl; 4 mg) | 1 | 5.23 |
| C and D | Formulation I | 72 | 0.639 |
| Rabbits | (20 µl; 4 mg) | | (average of two) |
| A and B Rabbits | Formulation I (10 µl; 2 mg) | 72 | 0.120 (average of two) |

As shown in Tables 7A and 7C, Novolimus with formulation I for eye injection with 2 mg to 4 mg dose efficiently delivered to eyes and generated Novolimus tissue concentration range from 135598 ng/g to 760 ng/g at day 1 and generated Novolimus tissue concentration range from 332 ng/g to 2 ng/g at day 72. The blood concentration was 5.23 ng/ml at day 1 and 0.120 ng/ml to 0.639 ng/ml at day 72. Both the tissue concentration and blood concentration were well within the therapeutically effective dose range.

TABLE 8A

Tissue Concentrations from Myolimus Formulation I Injection into Rabbit Eye

| Animal Group | Formulation (Dose in mg) | Time Point (Days) | Average Amount of Drug per Tissue Mass (ng/g) | | | |
|---|---|---|---|---|---|---|
| | | | Vitreous | Retina | Choroid | Sclera |
| J | Formulation I (20 µl; 3.5 mg) | 1 | 877017 | 2070 | 12099 | 24928 |
| F | Formulation I (23 µl; 4.0 mg) | 72 | 480072 | 9424 | 3114 | 209 |
| E | Formulation I (10 µl; 1.75 mg) | 72 | 275793 | 17254 | 22206 | 3293 |

TABLE 8B

Intraocular Pressure of Rabbit Eye from Myolimus Formulation I Injection

| Animal Group | Formulation (Dose in mg) | Intraocular Pressure (mm Hg) | | | |
|---|---|---|---|---|---|
| | | Day 1 | | Later Time Point (Day) | |
| | | pre-treatment* | post-treatment* | | |
| J | Formulation I (20 µl; 3.5 mg) | 14.5 | 29.0 | Day 2 25.0 | |
| F | Formulation I (23 µl; 4.0 mg) | 19.8 | 26.5 | Day 8/10 25.5 | Day 71 25.5 |
| E | Formulation I (10 µl; 1.75 mg) | 22.8 | 28.0 | Day 8/10 22.8 | Day 71 26.0 |

* Under anesthetic and dilation

TABLE 8C

Whole Blood Concentration from Myolimus Formulation I Injection into Rabbit Eye

| Animal Group | Formulation (Dose in mg) | Time Point (Days) | Whole Blood Concentration (ng/mL) |
|---|---|---|---|
| J | Formulation I (20 µl; 3.5 mg) | 1 | 1.25 |
| F | Formulation I (23 µl; 4.0 mg) | 72 | 0.177 |
| E | Formulation I (10 µl; 1.75 mg) | 72 | 0.155 |

As shown in Tables 8A and 8C, Myolimus with formulation I for eye injection with 1.75 mg to 4 mg dose efficiently delivered to eyes and generated Myolimus tissue concentration range from 877017 ng/g to 2070 ng/g at day 1 and generated Myolimus tissue concentration range from 480072 ng/g to 209 ng/g at day 72. The blood concentration was 1.25 ng/ml at day 1 and 0.155 ng/ml at day 72. Both the tissue concentration and blood concentration were well within the therapeutically effective dose range.

### Example 31: Preparation of m-TOR Organic Formulation With Self-Emulsiying Agent in Saline for Use in Eye Injection

Novolimus was dissolved in 95% PEG 300 NF with 5% Ethanol 190 Proof USP initially. Prior to injection into animal, this organic formulation with self-emulsifying agent was diluted with saline such that the final composition of this formulation was about 40 mg/mL of Novolimus in 63.3% PEG 300 NF, 3.3% Ethanol 190 Proof USP, and 33.3% saline. Myolimus was dissolved in 95% PEG 300 NF with 5% Ethanol 190 Proof USP initially. Prior to injection into animal, this organic formulation with self-emulsifying agent was diluted with saline such that the final composition of this formulation was about 4 mg/mL of Myolimus in 63.3% PEG 300 NF, 3.3% Ethanol 190 Proof USP, and 33.3% saline. These drug solutions were injected (20 to 50 µL) into rabbit’s eye and swine’s eye with the drug concentrations in target eye tissues and whole blood as shown in Tables 9A, 9C, 10A, and 10C. Drug concentrations in the eye tissues were measured using HPLC. Drug concentrations in the blood were measured using HPLC-MS. In addition, ocular pressure was measured during study as shown in Tables 9B and 10B. Ocular pressure was measured using a Model 30 ClassicT™ pneumatonometer.

TABLE 9A

Tissue Concentrations from Novolimus Formulation II Injection into Rabbit Eye and Swine Eye

| | | | Average Amount of Drug per Tissue Mass (ng/g) | | | |
|---|---|---|---|---|---|---|
| Animal Group | Formulation (Dose in mg) | Time Point (Days) | Vitreous | Retina | Choroid | Sclera |
| H rabbit | Formulation II (50 µl; 2 mg) | 1 | 60554 | 32338 | 3534 | 7207 |
| N rabbit | Formulation II (50 µl; 2 mg) | 37 | 1038 | 1176 | 591 | 236 |
| A rabbit | Formulation II (20 µl; 0.8 mg) | 1 | 80991 | 77291 | 22411 | 3782 |
| C rabbit | Formulation II (20 µl; 0.8 mg) | 36 | 80 | 228 | 265 | 64 |
| B rabbit | Formulation II (50 µl; 2 mg) | 1 | 167460 | 182312 | 85417 | 14610 |
| F swine | Formulation II (50 µl; 2 mg) | 1 | 70509 | 86662 | 45047 | 1150 |
| H swine | Formulation II (50 µl; 2 mg) | 36 | 314 | 469 | 455 | 70 |
| J swine | Formulation II (50 µl; 2 mg) | 72 | 5 | 3 | 12 | 10 |

TABLE 9B

Intraocular Pressure of Rabbit Eye from Novolimus Formulation II Injection

| Animal Group | Formulation (Dose in mg) | Intraocular Pressure (mm Hg) | | | |
|---|---|---|---|---|---|
| | | Day 1 | | Later Time Point (Day) | |
| | | pre-treatment* | post-treatment* | | |
| H rabbit | Formulation II (50 µl; 2 mg) | 14.0 | 36.5 | Day 2 15.3 | |
| N rabbit | Formulation II (50 µl; 2 mg) | 18.0 | 40.3 | Day 8/10 15.5 | Day 36 31.0 |

* Under anesthetic and dilation

TABLE 3C

Whole Blood Concentration from Novolimus Formulation II Injection into Rabbit Eye and Swine Eye

| Animal Group | Formulation (Dose in mg) | Time Point (Days) | Whole Blood Concentration (ng/mL) |
|---|---|---|---|
| H rabbit | Formulation II (50 µl; 2 mg) | 1 | 31.9 (average of two) |
| N rabbit | Formulation II (50 µl; 2 mg) | 37 | 4.88 |
| C rabbit | Formulation II (20 µl; 0.8 mg) | 36 | 0.895 (average of two) |
| H swine | Formulation II (50 µl; 2 mg) | 36 | 1.05 |

As shown in Tables 9A and 9C, Novolimus with formulation II for eye injection with 0.8 mg to 2 mg dose efficiently delivered to eyes and generated Novolimus tissue concentration range from 182312 ng/g to 1150 ng/g at day 1 and efficiently generated Novolimus tissue concentration range from 1176 ng/g to 64 ng/g at day 36 or day 37 and efficiently generated Novolimus tissue concentration range from 12 ng/g to 3 ng/g at day 72. The blood concentration was 31.9 ng/ml at day 1 and 0.895 ng/ml to 4.88 ng/ml at day 36 or day 37. Both the tissue concentration and blood concentration were well within the therapeutically effective dose range.

TABLE 10A

Tissue Concentrations from Myolimus Formulation II Injection into Rabbit Eye

| Animal Group | Formulation (Dose in mg) | Time Point (Days) | Average Amount of Drug per Tissue Mass (ng/g) | | | |
|---|---|---|---|---|---|---|
| | | | Vitreous | Retina | Choroid | Sclera |
| K | Formulation II (20 µl; 0.2 mg) | 1 | 33524 | 7583 | 6202 | 3525 |

TABLE 10B

Intraocular Pressure of Rabbit Eye from Myolimus Formulation II Injection into Rabbit Eye

| Animal Group | Formulation (Dose in mg) | Intraocular Pressure (mm Hg) | | |
|---|---|---|---|---|
| | | Day 1 | | Day 2 |
| | | pre-treatment* | post-treatment* | |
| K | Formulation II (20 µl; 0.2 mg) | 15.8 | 47.3 | 12.3 |

* Under anesthetic and dilation

TABLE 10C

Whole Blood Concentration from Myolimus Formulation III Injection into Rabbit Eye and Swine Eye (Studies NP100203* and NP100904)

| Animal Group | Formulation (Dose in mg) | Time Point (Days) | Whole Blood Concentration (ng/mL) |
|---|---|---|---|
| K | Formulation II (20 µl; 0.2 mg) | 1 | 0.652 |

As shown in Tables 10A and 10C, Myolimus with formulation II for eye injection with 0.2 mg dose efficiently delivered to eyes and generated Myolimus tissue concentration range from 33524 ng/g to 3525 ng/g at day 1 and with blood concentration of 0.652 ng/ml at day 1. Both the tissue concentration and blood concentration were well within the therapeutically effective dose range.

### Example 32: Preparation of m-TOR Formulation With Combination of Self-Emulsifying and Complexing Agents in Saline for Use in Eye Injection

Novolimus was dissolved in 36% PEG 400 NF with 9% absolute ethanol and 55% of an aqueous sodium phosphate buffer (pH 5) with Capitsol(35% w/v). The final Novolimus drug concentration was 2.7 mg/mL. Myolimus was dissolved in 45% PEG 400 NF with 10% absolute ethanol and 45% of an aqueous sodium phosphate buffer (pH 5) with Capitsol (35% w/v). The final Myolimus drug concentration was 0.9 mg/mL. These drug solutions were injected (50 µL) into rabbit’s eye and swine’s eye with the drug concentrations in target eye tissues and whole blood as shown in Tables 11A, 11C, 12A, and 12C. Drug concentrations in the eye tissues were measured using HPLC. Drug concentrations in the blood were measured using HPLC-MS. In addition, ocular pressure was measured during study as shown in Tables 11B and 12B. Ocular pressure was measured using a Model 30 Classic™ pneumatonometer.

TABLE 11A

Tissue Concentrations from Novolimus Formulation III Injection into Rabbit Eye and Swine Eye

| Animal Group | Formulation (Dose in mg) | Time Point (Days) | Average Amount of Drug per Tissue Mass (ng/g) | | | |
|---|---|---|---|---|---|---|
| | | | Vitreous | Retina | Choroid | Sclera |
| I rabbit | Formulation III (50 µl 0.14 mg) | 1 | 757 | 1591 | 2519 | 4022 |
| O rabbit | Formulation III (50 µl 0.14 mg) | 37 | 1.4 | 3.2 | 32 | 19 |
| G swine | Formulation III (50 µl 0.14 mg) | 1 | 10137 | 131076 | 32010 | 334 |
| I swine | Formulation III (50 µl 0.14 mg) | 36 | 57 | 39 | 60 | 14 |

TABLE 11B

Intraocular Pressure of Rabbit Eye from Novolimus Formulation III Injection

| Animal Group | Formulation (Dose in mg) | Intraocular Pressure (mm Hg) | | | |
|---|---|---|---|---|---|
| | | Day 1 | | Later Time Point (Day) | |
| | | pre-treatment* | post-treatment* | | |
| I rabbit | Formulation III (50 µl 0.14 mg) | 16.0 | 36.3 | Day 2 16.3 | |
| O rabbit | Formulation III (50 µl 0.14 mg) | 23.5 | 48.3 | Dav 8/10 20.0 | Dav 36 22.3 |

* Under anesthetic and dilation

TABLE 11C

Whole Blood Concentration from Novolimus Formulation III Injection into Rabbit Eye and Swine Eye

| Animal Group | Formulation (Dose in mg) | Time Point (Days) | Whole Blood Concentration (ng/mL) |
|---|---|---|---|
| I rabbit | Formulation III (50 µl 0.14 mg) | 1 | 5.75 (average of two) |
| O rabbit | Formulation III (50 µl 0.14 mg) | 37 | BQL† |
| G swine | Formulation III (50 µl 0.14 mg) | 1 | 5.23 |
| I swine | Formulation III (50 µl 0.14 mg) | 36 | BQL† |

† < 5 ng/mL (Novolimus)

As shown in Tables 11A and 11C, Novolimus with formulation III for eye injection with 0.14 mg dose efficiently delivered to eyes and generated Novolimus tissue concentration range from 131076 ng/g to 334 ng/g at day 1 and efficiently generated Novolimus tissue concentration range from 60 ng/g to 14 ng/g at day 36 or day 37. The blood concentration was 5.23 ng/ml to 5.75 ng/ml at day 1. Both the tissue concentration and blood concentration were well within the therapeutically effective dose range.

TABLE 12A

Tissue Concentrations from Myolimus Formulation III Injection into Rabbit Eye

| Animal Group | Formulation (Dose in mg) | Time Point (Days) | Average Amount of Drug per Tissue Mass (ng/g) | | | |
|---|---|---|---|---|---|---|
| | | | Vitreous | Retina | Choroid | Sclera |
| L | Formulation III (50 µl 0.05 mg) | 1 | 843 | 5314 | 5033 | 18923 |
| D | Formulation III (50 µl 0.05 mg) | 36 | 6 | 57 | 81 | 38 |

TABLE 12B

Intraocular Pressure of Rabbit Eye from Myolimus Formulation III Injection

| Animal Group | Formulation (Dose in mg) | Time Point (Days) | Intraocular Pressure (mm Hg) | | |
|---|---|---|---|---|---|
| | | | Day 1 | | Day 2 |
| | | | pre-treatment* | post-treatment* | |
| L | Formulation III (50 µl 0.05 mg) | 1 | 16.3 | 38.3 | 13.3 |

* Under anesthetic and dilation

TABLE 12C

Whole Blood Concentration from Myolimus Formulation III Injection into Rabbit Eye

| Animal Group | Formulation (Dose in mg) | Time Point (Days) | Whole Blood Concentration (ng/mL) |
|---|---|---|---|
| L | Formulation III (50 µl 0.05 mg) | 1 | 0.708 |
| D | Formulation III (50 µl 0.05 mg) | 36 | BQL^{†} |

† < 0.25 ng/mL (Myolimus)

As shown in Tables 12A and 12C, Myolimus with formulation III for eye injection with 0.05 mg dose efficiently delivered to eyes and generated Myolimus tissue concentration range from 18923 ng/g to 843 ng/g at day 1 and range from 81 ng/g to 6 ng/g at day 36 and with blood concentration of 0.708 ng/ml at day 1. Both the tissue concentration and blood concentration were well within the therapeutically effective dose range.

### Example 33: Preparation of Direct Factor Xa Inhibitor Injection Formulation for Ophthalmic Application

Direct factor Xa inhibitor (such as Apixaban or Rivaroxaban) at target concentration in USP proof alcohol dissolves in PEG and/or copolymer of PEG with USP proof alcohol. The drug solution is injected (10 to 20 µL) into eye. This formulation, following local application into the eye, is sufficient to provide a therapeutically effective dose of the direct factor Xa inhibitor at day 1 through day 72 or more. Some formulations are sufficient to generate a therapeutically effective dose of the direct factor Xa lasting through 30 days locally, 60 days, 90 days, and even 6 months while below in the systemic circulation therapeutic levels at or after day 1, at day 2, at day 3, at day 7, and even longer from introduction in the eye.

### Example 34: Preparation of Direct Factor Xa Inhibitor Suspension Formulation for Ophthalmic Application with Excipients

Direct factor Xa inhibitor (such as Apixaban or Rivaroxaban) at target concentration formulated as ophthalmic suspension or solution that contains excipients (all, less, or more) such as suspending agents such as carboxymethylcellulose, hypromellose, povidone, buffers, preservatives, surfactants, caffeine, nicotinamide derivatives and complex/encapsulating agents such as derivatives of cyclodextrin (hydroxylpropyl cyclodextrin, captisol). This formulation can use as short release or longer release depending on the delivery method. Some of the delivery methods are sufficient to generate a therapeutically effective dose of the direct factor Xa lasting through 30 days locally, 60 days, 90 days, and even 6 months while negligible in the systemic circulation at day 1, at day 2, at day 3, at day 7, and even longer.

### Example 35: Preparation of Direct Factor Xa Inhibitor Lipid Emulsions Formulation For Ophthalmic Application with Excipients

Direct Factor Xa inhibitor (such as Apixaban or Rivaroxaban) at target concentration formulated as lipid emulsions (all, less, or more) such as medium chain triglycerides with polymeric surface modifiers such as chitosan (CS) and poloxamer 407 (P407) with other components such as surfactants, caffeine, and/or nicotinamide derivatives. This formulation can use as short release or longer release depending on the delivery method. This formulation, following local application into the eye, is sufficient to provide a therapeutically effective dose of the direct factor Xa inhibitor at day 1 through day 72. Some of the delivery methods are sufficient to generate a therapeutically effective dose of the direct factor Xa lasting through 30 days locally, 60 days, 90 days, and even 6 months while negligible in the systemic circulation at day 1, at day 2, at day 3, at day 7, and even longer.

### Example 36: Preparation of Direct Factor Xa Inhibitor Combined With m-TOR Suspension Formulation for Ophthalmic Application with Excipients

Direct Factor Xa inhibitor (such as Apixaban or Rivaroxaban) at target concentration formulated with m-TOR (such as Novolimus or Myolimus ) as ophthalmic suspension or solution that contains excipients (all, less, or more), suspending agents such as carboxymethylcellulose, hypromellose, povidone, buffers, preservatives, surfactants, caffeine, nicotinamide derivatives and complex/encapsulating agents such as derivatives of cyclodextrin (hydroxylpropyl cyclodextrin, captisol). This formulation can use as short release or longer release depending on the delivery method. Some of the delivery method applies into the eye that follows the local dose generates sufficient therapeutically effective dose of the direct factor Xa inhibitor and m-TOR at day 1 through day 72. Some of formulation generates sufficient therapeutically effective dose of the direct factor Xa inhibitor and m-TOR lasting through 30 days locally, 60 days, 90 days, and even 6 months while below the systemic circulation therapeutic level at day 1, at day 2, at day 3, or at day 7.

### Example 37: Preparation of Direct Factor Xa Inhibitor Combined With m-TOR Lipid Emulsions Formulation for Ophthalmic Application with Excipients

Direct Factor Xa inhibitor (such as Apixaban or Rivaroxaban) at target concentration formulated with m-TOR (such as Novolimus or Myolimus) in lipid emulsions (all, less, or more) such as medium-chain medium chain triglycerides with polymeric surface modifiers such as chitosan (CS) and poloxamer 407 (P407) with other components such as surfactants, caffeine, nicotinamide derivatives. This formulation can use as short release or longer release depending on the delivery method. Some of the delivery method applies into the eye that follows the local dose generates sufficient therapeutically effective dose of the direct factor Xa inhibitor and m-TOR at day 1 through day 72. Some of formulation generates sufficient therapeutically effective dose of the direct factor Xa inhibitor and m-TOR lasting through 30 days locally, 60 days, 90 days, and even 6 months below the systemic circulation therapeutic level at day 1, at day 2, at day 3, at day 7.

### Example 38: Preparation of Direct Factor Xa Inhibitor Combined With Aflibercept Formulation For Ophthalmic Application with Excipients

Direct Factor Xa inhibitor (such as Apixaban or Rivaroxaban) at target concentration formulated (such as Apixaban or Rivaroxaban) will be formulated with Eylea (aflibercept) with excipients (all, less, or more) such as buffers, surfactant (such as tween), NaCl, sugar (such as trehalose), amino acid (such as arginine) and complex/encapsulating agents such as derivatives of cyclodextrin (hydroxylpropyl cyclodextrin, captisol). This formulation can use as short release or longer release depending on the delivery method. Some of the delivery method applies into the eye that follows the local dose generates sufficient therapeutically effective dose of the direct factor Xa inhibitor and aflibercept at day 1 through day 72. Some of formulation generates sufficient therapeutically effective dose of the direct factor Xa inhibitor and aflibercept lasting through 30 days locally, 60 days, 90 days, and even 6 months while below the systemic circulation therapeutic level at day 1, at day 2, at day 3, or at day 7.

### Example 39: Preparation of Direct Factor Xa Inhibitor Combined With Ranibizumab Formulation for Ophthalmic Application with Excipients

Direct Factor Xa inhibitor (such as Apixaban or Rivaroxaban) at target concentration formulated with Ranibizumab with excipients (all, less, or more) such as buffers, surfactant (such as tween), NaCl, sugar (such as trehalose), amino acid (such as arginine) and complex/encapsulating agents such as derivatives of cyclodextrin (hydroxylpropyl cyclodextrin, captisol). This formulation can use as short release or longer release depending on the delivery method. Some of the delivery method applies into the eye that follows the local dose generates sufficient therapeutically effective dose of the direct factor Xa inhibitor and Ranibizumab at day 1 through day 72. Some of the delivery method generates sufficient therapeutically effective dose of the direct factor Xa inhibitor and Ranibizumab lasting through 30 days locally, 60 days, 90 days, and even 6 months while below the systemic circulation therapeutic level at day 1, at day 2, at day 3, or at day 7.

### Example 40: Preparation of Direct Factor Xa Inhibitor Eye Drop Formulation for Ophthalmic Application with Excipients

Direct Factor Xa inhibitor (such as Apixaban or Rivaroxaban) at target concentration formulated in sterile water and/or artificial tears and/or buffer that contains (all, less, or more) suspending agents such as carboxymethylcellulose, hypromellose, povidone, complex/ encapsulating agents such as derivatives of cyclodextrin (hydroxylpropyl cyclodextrin, captisol) and other components such as surfactants, caffeine, nicotinamide derivatives as needed. This formulation applies into the eye that follows the local dose generates sufficient therapeutically effective dose of the direct factor Xa inhibitor while negligible in the systemic circulation.

### Example 41: Preparation of Direct Factor Xa Inhibitor Eye Drop Polymeric Micelles Formulation for Ophthalmic Application with Excipients

Direct Factor Xa inhibitor (such as Apixaban or Rivaroxaban) at target concentration formulated in sterile water and/or artificial tears and/or buffer that contains (all, less, or more) polymeric micelles such as poly(ethylene oxide)-poly(propylene oxide)-poly(ethylene oxide) (PEO-PPO-PEO) and other components such as surfactants, caffeine, nicotinamide derivatives. This formulation applies into the eye that follows the local dose generates sufficient therapeutically effective dose of the direct factor Xa inhibitor while negligible in the systemic circulation.

### Example 42: Preparation of Anticoagulant (Rivaroxaban, Argatroban, or Apixaban) Drug Coated Contact Lens

Poly (methyl methacrylate) or Poly (2-hydroxyethyl methacrylate) or similar polymers is dissolved into proper solvent at room temperature and vortex or stir until the polymer have been uniformly dissolved/dispersed. Anticoagulant (rivaroxaban, argatroban, or Apixaban or combinations) is dissolved into solvent at room temperature and vortex/ or stir until the drug is uniformly dissolved/dispersed. Each polymer solution and each drug solution are combined at a target weight ratio according to the target drug dose for each drug. This mixed solution can be coated on the commercially available contact lens or commercially available therapeutically soft lens or commercially available therapeutically scleral lens by dipping method or spray method. If by a spray method, a microprocessor-controlled ultrasonic sprayer will be used to coat each of the commercially available contact lens or commercially available therapeutically soft lens or commercial available therapeutically scleral lens until a uniformly dispense with each of the drug/polymer matrix . After coating, the contact lens will be placed in a vacuum chamber to remove the solvent. The contact lens is then packaged into a pouch. The pouches are sterilized. This drug eluting lens can be used either at night or daytime depending on the target using drug release profile. This drug delivery system comprising anticoagulant (rivaroxaban, argatroban, or Apixaban) coated contact lens could have a dispersed particle size less than about 200 nm, an anticoagulant (rivaroxaban, argatroban, or Apixaban) is nanoencapsulated in a material and is capable of diffusion into and migration through contact lens and into the post-lens tear film when contact lens is placed on the eye. This contact lens applies into the eye that follows the local dose generates sufficient therapeutically effective dose of the direct factor Xa inhibitor and/or the direct factor IIa inhibitor while below the systemic circulation therapeutic level.

### Example 43: Preparation of Anticoagulant (Rivaroxaban, Argatroban, or Apixaban) Combined with Metformin Drug Coated Contact Lens

Poly (methyl methacrylate) or Poly (2-hydroxyethyl methacrylate) or similar polymer is dissolved into proper solvent at room temperature and vortex or stir until the polymer have been uniformly dissolved/dispersed. Anticoagulant (rivaroxaban, argatroban, or Apixaban or combinations) and metformin or metformin.HCl is dissolved into proper solvent at room temperature and vortex/ or stir until the drug is uniformly dissolved/dispersed. Each polymer solution and each drug solution are combined at a target weight ratio according to the target drug dose for each drug. This mixed solution can be coated on the commercially available contact lens or commercially available therapeutically soft lens or commercially available therapeutically scleral lens by dipping method or spray method. If by a spray method, a microprocessor-controlled ultrasonic sprayer will be used to coat each of the commercially available contact lens or commercially available therapeutically soft lens or commercially available therapeutically scleral lens until a uniformly dispense with each of the drug/polymer matrix. After coating, the contact lens will be placed in a vacuum chamber to remove the solvent. The contact lenses are then packaged into a pouch. The pouches are sterilized. This drug eluting lens can be used either at night or daytime depending on the target using drug release profile. This drug delivery system comprising anticoagulant (rivaroxaban, argatroban, or Apixaban) combined with metformin coated contact lens could have a dispersed particle size less than about 200 nm, anticoagulant (rivaroxaban, argatroban, or Apixaban) with metformin are nanoencapsulated in a material and are capable of diffusion into and migration through contact lens and into the post-lens tear film when contact lens is placed on the eye. This contact lens applies into the eye that follows the local dose generates sufficient therapeutically effective dose of the direct factor Xa inhibitor/ the direct factor IIa inhibitor/metformin while negligible in the systemic circulation.

### Example 44: Preparation of Direct Factor Xa Inhibitor Apixaban and Direct Factor IIa Inhibitor Argatroban Combined with 25% Capitsol in Citrate for Application in Ophthalmic Injection or Eye Drops

6600 mg Capitsol was dissolved in 15 mM sodium citrate in a 25.0 mL volumetric flask and the solution was sonicated and mixed until clear. This solution labeled as 25% (250 mg/mL) Captisol (Solution A). 100 mg of apixaban was weighed into a separate vial and 20.0 mL of the 25% Captisol solution(Solution A) prepared above was added. This 20 mL of apixaban solution was sonicated with heat at 40° C. for approximately 30 minutes to maximally dissolve the apixaban. This “saturated apixaban solution” was cooled and filtered to create a clear solution of apixaban in 25% Captisol (Solution B). 51 mg of argatroban was added to a separate vial, and 10 mL of the saturated apixaban solution(Solution B) prepared above was added to the same vial. The final combination solution was sonicated with heat at 40° C. for approximately 30 minutes until clear. The cooled solution was filtered, and pH as measured was pH 7.5. The concentration of the anticoagulants were measured as 1.5 mg/mL of apixaban and 5.0 mg/mL of argatroban. These drug solutions are injected (10 to 50 µL) into rabbit’s eye or swine’s eye with the drug concentrations in target eye tissues and whole blood. Drug concentrations in the eye tissues and in the blood is measured. In addition, ocular pressure is measured during study.

If formulated as eye drops, other than preservatives, the other inactive ingredient may be added to adjust drug concentration and tonicity such as buffers for adjusting pH, drug stabilizers such as anti-oxidants, solubility enhancers, viscosity adjusters, and the like. These inactive ingredients include but not limit to benzalkonium chloride, chlorobutanol, sodium dehydroacetate, phenethyl alcohol, benzethonium chloride, and the like; Boric Acid, Edetate Disodium, Potassium Chloride, Sodium Borate, Sodium Chloride and the like; methyl cellulose, hydroxyethyl cellulose, carboxymethyl cellulose, hydroxypropyl methyl cellulose, polyvinyl alcohol and the like; polyoxyethylene hydrogenated castor oil, polyethylene glycol, polysorbate 80, polyoxyethylene monostearate and the like; sodium edetate and citric acid, sodium edetate and sodium bisulfite and the like.

This formulation applies into the eye that follows the local dose generates sufficient therapeutically effective dose of the drug while below the systemic circulation therapeutic level.

### Example 45: Preparation of Direct Factor Xa Inhibitor Apixaban and Direct Factor IIa Inhibitor Argatroban Combined with 30% Capitsol in Citrate for Application in Ophthalmic Injection or Eye Drops

7900 mg Capitsol was dissolved in 15 mM sodium citrate in a 25.0 mL volumetric flask, and the solution was sonicated and mixed until clear. This solution labeled as 30% (300 mg/mL) Captisol(Solution C). 100 mg of apixaban was weighed into a separate vial and 20.0 mL of the 30% Captisol solution (Solution C) prepared above was added. This 20 mL of apixaban solution was sonicated with heat at 40° C. for approximately 30 minutes to maximally dissolve the apixaban. This “saturated apixaban solution” was cooled and filtered to create a clear solution of apixaban in 30% Captisol(Solution D). 51 mg of argatroban was added to a separate vial, and 10 mL of the saturated apixaban solution prepared above (Solution D) was added to the same vial. The final combination solution was sonicated with heat at 40° C. for approximately 30 minutes until clear. The cooled solution was filtered, and pH as measured was pH 7.3. The concentration of the anticoagulants were measured as 2.0 mg/mL of apixaban and 5.0 mg/mL of argatroban. These drug solutions are injected (10 to 50 µL) into rabbit’s eye or swine’s eye with the drug concentrations in target eye tissues and whole blood. Drug concentrations in the eye tissues and in the blood is measured. In addition, ocular pressure is measured during study.

If formulated as eye drops, other than preservatives, the other inactive ingredient may be added to adjust drug concentration and tonicity such as buffers for adjusting pH, drug stabilizers such as anti-oxidants, solubility enhancers, viscosity adjusters, and the like. These inactive ingredients include but not limit to benzalkonium chloride, chlorobutanol, sodium dehydroacetate, phenethyl alcohol, benzethonium chloride, and the like; Boric Acid, Edetate Disodium, Potassium Chloride, Sodium Borate, Sodium Chloride and the like; methyl cellulose, hydroxyethyl cellulose, carboxymethyl cellulose, hydroxypropyl methyl cellulose, polyvinyl alcohol and the like; polyoxyethylene hydrogenated castor oil, polyethylene glycol, polysorbate 80, polyoxyethylene monostearate and the like; sodium edetate and citric acid, sodium edetate and sodium bisulfite and the like.

This formulation applies into the eye that follows the local dose generates sufficient therapeutically effective dose of the drug while negligible in the systemic circulation.

### Example 46: Preparation of Direct Factor Xa Inhibitor Apixaban and Direct Factor IIa Inhibitor Argatroban Combined with 40% Capitsol in Citrate for Application in Ophthalmic Injection or Eye Drops

10,560 mg Capitsol was dissolved in 15 mM sodium citrate in a 25.0 mL volumetric flask, and the solution was sonicated and mixed until clear. This solution labeled as 40% (400 mg/mL) Captisol(Solution E). 100.2 mg of apixaban was weighed into a separate vial and 20.0 mL of the 40% Captisol solution(Solution E) prepared above was added. This 20 mL of apixaban solution was sonicated with heat at 40° C. for approximately 30 minutes to maximally dissolve the apixaban. This “saturated apixaban solution” was cooled and filtered to create a clear solution of apixaban in 40% Captisol (Solution F). 51 mg of argatroban was added to a separate vial, and 10 mL of the saturated apixaban solution(Solution F) prepared above was added to the same vial. The final combination solution was sonicated with heat at 40° C. for approximately 30 minutes until clear. The cooled solution was filtered, and pH as measured was pH 6.9. The concentration of the anticoagulants was measured as 3.2 mg/mL of apixaban and 5.0 mg/mL of argatroban. These drug solutions are injected (10 to 50 µL) into rabbit’s eye or swine’s eye with the drug concentrations in target eye tissues and whole blood. Drug concentrations in the eye tissues and in the blood is measured. In addition, ocular pressure is measured during study.

If formulated as eye drops, other than preservatives, the other inactive ingredient may be added to adjust drug concentration and tonicity such as buffers for adjusting pH, drug stabilizers such as anti-oxidants, solubility enhancers, viscosity adjusters, and the like. These inactive ingredients include but not limit to benzalkonium chloride, chlorobutanol, sodium dehydroacetate, phenethyl alcohol, benzethonium chloride, and the like; Boric Acid, Edetate Disodium, Potassium Chloride, Sodium Borate, Sodium Chloride and the like; methyl cellulose, hydroxyethyl cellulose, carboxymethyl cellulose, hydroxypropyl methyl cellulose, polyvinyl alcohol and the like; polyoxyethylene hydrogenated castor oil, polyethylene glycol, polysorbate 80, polyoxyethylene monostearate and the like; sodium edetate and citric acid, sodium edetate and sodium bisulfite and the like.

This formulation applies into the eye that follows the local dose generates sufficient therapeutically effective dose of the drug while negligible in the systemic circulation.

### Example 47: Preparation of Direct Factor Xa Inhibitor Apixaban and Direct Factor IIa Inhibitor Argatroban Combined with 50% Captisol in Citrate for Application in Ophthalmic Injection or Eye Drops

13,200 mg Capitsol was dissolved in 15 mM sodium citrate in a 25.0 mL volumetric flask, and the solution was sonicated and mixed until clear. This solution labeled as 50% (500 mg/mL) Captisol(Solution G). 120.2 mg of apixaban was weighed into a separate vial and 20.0 mL of the 50% Captisol solution(Solution G) prepared above was added. This 20 mL of apixaban solution was sonicated with heat at 40° C. for approximately 30 minutes to maximally dissolve the apixaban. This “saturated apixaban solution” was cooled and filtered to create a clear solution of apixaban in 50% Captisol(Solution H). 62 mg of argatroban was added to a separate vial, and 10 mL of the saturated apixaban solution (Solution H) prepared above was added to the same vial. The final combination solution was sonicated with heat at 40° C. for approximately 30 minutes until clear. The cooled solution was filtered, and pH as measured was pH 7.6. The concentration of the anticoagulants were measured as 3.7 mg/mL of apixaban and 6.0 mg/mL of argatroban. These drug solutions are injected (10 to 50 µL) into rabbit’s eye or swine’s eye with the drug concentrations in target eye tissues and whole blood. Drug concentrations in the eye tissues and in the blood is measured. In addition, ocular pressure is measured during study.

If formulated as eye drops, other than preservatives, the other inactive ingredient may be added to adjust drug concentration and tonicity such as buffers for adjusting pH, drug stabilizers such as antioxidants, solubility enhancers, viscosity adjusters, and the like. These inactive ingredients include but not limit to benzalkonium chloride, chlorobutanol, sodium dehydroacetate, phenethyl alcohol, benzethonium chloride, and the like; Boric Acid, Edetate Disodium, Potassium Chloride, Sodium Borate, Sodium Chloride and the like; methyl cellulose, hydroxyethyl cellulose, carboxymethyl cellulose, hydroxypropyl methyl cellulose, polyvinyl alcohol and the like; polyoxyethylene hydrogenated castor oil, polyethylene glycol, polysorbate 80, polyoxyethylene monostearate and the like; sodium edetate and citric acid, sodium edetate and sodium bisulfite and the like.

This formulation applies into the eye that follows the local dose generates sufficient therapeutically effective dose of the drug while negligible in the systemic circulation.

### Example 48: Preparation of Direct Factor Xa Inhibitor Apixaban and Direct Factor IIa Inhibitor Argatroban Combined with Azelastine (Antihistamine) in Capitsol in Citrate for Application in Ophthalmic Injection or Eye Drops

In a formulation using example 18, one additional drug of azelastine hydrochloride can be added to the final vial. All the excipients and drugs can be varied within a range.

10,560 mg Capitsol was dissolved in 15 mM sodium citrate in a 25.0 mL volumetric flask, and the solution was sonicated and mixed until clear. This solution labeled as 40% (400 mg/mL) Captisol(Solution E). 100.2 mg of apixaban was weighed into a separate vial and 20.0 mL of the 40% Captisol solution(Solution E) prepared above was added. This 20 mL of apixaban solution was sonicated with heat at 40° C. for approximately 30 minutes to maximally dissolve the apixaban. This “saturated apixaban solution” was cooled and filtered to create a clear solution of apixaban in 40% Captisol (Solution F). Two drugs (51 mg of argatroban and 1.0 mg of Azelastine hydrochloride) are weighed and added into the same vial, and finally 10 mL of the saturated apixaban solution (Solution F) prepared above is added to the same vial. The final 3 drug combination solution is sonicated with heat if needed until clear. The cooled solution is filtered, and pH is measured and adjusted to the suitable physiological pH’s. The concentration of the anticoagulants are projected to be at 3.2 mg/mL of apixaban and 5.0 mg/mL of argatroban, and azelastine is confirmed around 0.1 mg/mL. These drug solutions are injected (10 to 50 µL) into rabbit’s eye or swine’s eye with the drug concentrations in target eye tissues and whole blood. Drug concentrations in the eye tissues and in the blood is measured. In addition, ocular pressure is measured during study.

If formulated as eye drops, other than preservatives, the other inactive ingredient may be added to adjust drug concentration and tonicity such as buffers for adjusting pH, drug stabilizers such as antioxidants, solubility enhancers, viscosity adjusters, and the like. These inactive ingredients include but not limit to benzalkonium chloride, chlorobutanol, sodium dehydroacetate, phenethyl alcohol, benzethonium chloride, and the like; Boric Acid, Edetate Disodium, Potassium Chloride, Sodium Borate, Sodium Chloride and the like; methyl cellulose, hydroxyethyl cellulose, carboxymethyl cellulose, hydroxypropyl methyl cellulose, polyvinyl alcohol and the like; polyoxyethylene hydrogenated castor oil, polyethylene glycol, polysorbate 80, polyoxyethylene monostearate and the like; sodium edetate and citric acid, sodium edetate and sodium bisulfite and the like.

This formulation applies into the eye that follows the local dose generates sufficient therapeutically effective dose of the drug while negligible in the systemic circulation.

### Example 49: Preparation of Direct Factor Xa Inhibitor Apixaban and Direct Factor IIa Inhibitor Argatroban Combined with Hydoxychloroquine in Captisol in Citrate for Application in Ophthalmic Injection or Eye Drops

In a formulation using example 18, one additional drug of hydroxychloroquine sulfate can be added to the final vial. All the excipients and drugs can be varied within a range.

10,560 mg Capitsol was dissolved in 15 mM sodium citrate in a 25.0 mL volumetric flask, and the solution was sonicated and mixed until clear. This solution labeled as 40% (400 mg/mL) Captisol (Solution E). 100.2 mg of apixaban was weighed into a separate vial and 20.0 mL of the 40% Captisol solution(Solution E) prepared above was added. This 20 mL of apixaban solution was sonicated with heat at 40° C. for approximately 30 minutes to maximally dissolve the apixaban. This “saturated apixaban solution” was cooled and filtered to create a clear solution of apixaban in 40% Captisol (Solution F). In a separate vial, two drugs(51 mg of argatroban and 15 mg of hydroxychloroquine sulphate ) can be weighed and added to this single vial and finally 10 mL of the saturated apixaban solution (Solution F) prepared above is added to this same vial. The final 3 drug combination solution is sonicated with heat if needed until clear. The cooled solution is filtered, and pH is measured and adjusted to the suitable physiological pH’s. The concentration of the anticoagulants are projected to be at 3.2 mg/mL of apixaban and 5.0 mg/mL of argatroban, and 1.5 mg/mL of hydroxychloroquine sulphate. These drug solutions are injected (10 to 50 µL) into rabbit’s eye or swine’s eye with the drug concentrations in target eye tissues and whole blood. Drug concentrations in the eye tissues and in the blood is measured. In addition, ocular pressure is measured during study.

If formulated as eye drops, other than preservatives, the other inactive ingredient may be added to adjust drug concentration and tonicity such as buffers for adjusting pH, drug stabilizers such as anti-oxidants, solubility enhancers, viscosity adjusters, and the like. These inactive ingredients include but not limit to benzalkonium chloride, chlorobutanol, sodium dehydroacetate, phenethyl alcohol, benzethonium chloride, and the like; Boric Acid, Edetate Disodium, Potassium Chloride, Sodium Borate, Sodium Chloride and the like; methyl cellulose, hydroxyethyl cellulose, carboxymethyl cellulose, hydroxypropyl methyl cellulose, polyvinyl alcohol and the like; polyoxyethylene hydrogenated castor oil, polyethylene glycol, polysorbate 80, polyoxyethylene monostearate and the like; sodium edetate and citric acid, sodium edetate and sodium bisulfite and the like.

This formulation applies into the eye that follows the local dose generates sufficient therapeutically effective dose of the drug while negligible in the systemic circulation.

### Example 50: Preparation of Direct Factor Xa Inhibitor Apixaban and Direct Factor IIa Inhibitor Argatroban Combined with Anti-VEGF (Brolucizumab, Aflibercept or Ranibizumab or Pegaptanib Sodium or Bevacizumab) in Capitsol in Citrate for Application in Ophthalmic Injection or Eye Drops

In a formulation as in example 18, one additional drug of commercial medications such as brolucizumab or aflibercept or ranibizumab or pegaptanib sodium or bevacizumab can be added to the final vial. All the excipients and drugs can be varied within a range.

10,560 mg Capitsol is dissolved in 15 mM sodium citrate in a 25.0 mL volumetric flask, and the solution was sonicated and mixed until clear. This solution labeled as 40% (400 mg/mL) Captisol(Solution E). 100.2 mg of apixaban was weighed into a separate vial and 20.0 mL of the 40% Captisol solution(Solution E) prepared above was added. This 20 mL of apixaban solution was sonicated with heat at 40° C. for approximately 30 minutes to maximally dissolve the apixaban. This “saturated apixaban solution” was cooled and filtered to create a clear solution of apixaban in 40% Captisol (Solution F). In a separate single vial, 51 mg of argatroban and 1201 mg of brolucizumab or 401 mg of aflibercept or 101 mg of ranibizumab or 34 mg of pegaptanib sodium or 251 mg bevacizumab is weighed into the same vial, and finally 10 mL of the saturated apixaban solution prepared above is added to the vial. The final 3 drug combination solution is sonicated with heat if needed until clear. The cooled solution is filtered, and pH is measured and adjusted to the suitable physiological pH’s. The concentration of the anticoagulants are projected to be at 3.2 mg/mL of apixaban and 5.0 mg/mL of argatroban, and other drugs is confirmed at around 120 mg/mL of brolucizumab or 40 mg/mL of aflibercept or 10 mg/mL of ranibizumab or 3.3 mg/mL of pegaptanib sodium or 25 mg/mL bevacizumab. These drug solutions are injected (10 to 50 µL) into rabbit’s eye or swine’s eye with the drug concentrations in target eye tissues and whole blood. Drug concentrations in the eye tissues and in the blood is measured. In addition, ocular pressure is measured during study.

If formulated as eye drops, other than preservatives, the other inactive ingredient may be added to adjust drug concentration and tonicity such as buffers for adjusting pH, drug stabilizers such as anti-oxidants, solubility enhancers, viscosity adjusters, and the like. These inactive ingredients include but not limit to benzalkonium chloride, chlorobutanol, sodium dehydroacetate, phenethyl alcohol, benzethonium chloride, and the like; Boric Acid, Edetate Disodium, Potassium Chloride, Sodium Borate, Sodium Chloride and the like; methyl cellulose, hydroxyethyl cellulose, carboxymethyl cellulose, hydroxypropyl methyl cellulose, polyvinyl alcohol and the like; polyoxyethylene hydrogenated castor oil, polyethylene glycol, polysorbate 80, polyoxyethylene monostearate and the like; sodium edetate and citric acid, sodium edetate and sodium bisulfite and the like.

This formulation applies into the eye that follows the local dose generates sufficient therapeutically effective dose of the drug while negligible in the systemic circulation.

### Example 51: Preparation of Direct Factor Xa Inhibitor Apixaban and Direct Factor IIa Inhibitor Argatroban and m-TOR Self-Assembly Nanomicellar Formulation for Ophthalmic Drug Delivery

Anticoagulant (Apixaban, Argatroban, Rivaroxaban etc.) and m-TOR are dispersed within nano scale range from 10 nm to 200 nm. The most used carriers in nanomedicine include but not limit to lipids (liposomes), proteins (albumin nanoparticles), cyclic oligosaccharides (cyclodextrins), synthetic polymers (polymeric micelles, dendrimers, hydrogel), castor oil, octoxylnol, and inorganic compounds (cerium oxide nanoparticles). This nanocarriers increase the solubility of hydrophobic drugs, their capability to provide sustained drug release with reduced toxicity and improved efficacy, their ability to prolong drug retention time and enhancement of drug penetration through ocular barriers, and their proficiency to direct drugs to specific tissues and cells.

Self-assembly of anticoagulant(Apixaban, Argatroban, Rivaroxaban etc.) and m-TOR can be formulated nanomicelles at target concentration using amphiphilic molecules such as surfactants, polymer amphiphiles, amphiphilic PEG-Hydrodenated castor oil, Octyxonal-40 amphiphilic polymers and some lipid molecules, which containing both hydrophilic and hydrophobic components. This allows entrapment of hydrophobic drugs. Anticoagulant(Apixaban, Argatroban, Rivaroxaban etc.) and m-TOR can be formulated in the core of the nanomicelles using solvent evaporation-film rehydration method. PEG-Hydrogenated castor oil and Octoxynol-40 are weighed and dissolved in ethanol or methanol at 1%(w/w) to 5%(w/w) in a round bottle flask and is mixed above critical micellar concentration (CMC), then add 0.1%(w/w) to 0.5%(w/w) of ethanolic or methanolic solution of anticoagulant (Apixaban, Argatroban, Rivaroxaban etc.) and m-TOR in the same round bottle flask. The organic solvent was evaporated at a high speed under vacuum to obtain a thin solid film of polymer and drugs. The resulting film was rehydrated with HPLC grade water and sonicated. This solution is followed by filtration through a 0.22 µm or less nylon syringe filter or the similar filter system. This results anticoagulant (Apixaban, Argatroban, Rivaroxaban etc.) and m-TOR in nanomicelles in water solution. Some of the delivery method applies into the eye that follows the local dose generates sufficient therapeutically effective dose of the direct factor Xa inhibitor and/or the director factor IIa inhibitor and m-TOR at day 1 through day 72. Some of the delivery method generates sufficient therapeutically effective dose of the direct factor Xa inhibitor and/or director factor IIa inhibitor and m-TOR lasting through 30 days locally, 60 days, 90 days, and even 6 months while negligible in the systemic circulation at day 1, at day 2, at day 3, at day 7, and even longer.

### Example 52: Preparation of Anticoagulant Apixaban Crystal Fine Particles Composition in PEG 300 for Ophthalmic Injection

10 mg of apixaban powder was weighed into a vial and 50 µL of 190 proof USP grade ethanol was added first in the same vial, then 950 uL of PEG 300 (polyethylene glycol) was added to solubilize the drug. The vial was sonicated with 40° C. heat for 30 minutes and then was placed in the 40° C. oven overnight for 20 hours. The cloudy solution was transferred to a polyethylene centrifuge tube and centrifuged for 15 minutes to pelletize the undissolved apixaban. The clear supernatant was analyzed by HPLC and the concentration was found to be 6200 µg/mL apixaban in 95% PEG 300 solution. This clear solution is ready for eye injection. After injection into eyes, these drugs will form a crystal fine particle in PEG 300 and the drug has extended release profiles. The crystal fine particles were verified by following.

100 µL of the clear PEG 300 solution was added to a vial with 4.3 mL of Dulbecco’s phosphate buffered saline to mimic the ophthalmic vitreous humor. The solution immediately cleared upon mixing. The solution stands overnight for 24 hours at room temperature. Upon close inspection, the apixaban had precipitated producing a small carpet of crystals on the bottom of the glass vial. The solution was then centrifuged, and the supernatant was analyzed by HPLC. The concentration was found to be 94 µg/mL of apixaban compared to the theoretical concentration in the PBS at 141 µg/mL. Therefore 67% of the apixaban was in solution and 33 % had precipitated out to crystalline form.

This clear periocular injection which late forms fine particles containing Apixaban and enables Apixaban to deliver to the posterior segments. Apixaban can be efficiently delivered to the posterior segments (such as a retina, a choroid and an optic nerve) while scarcely injuring ophthalmic tissues by administering the fine particles containing Apixaban periocularlly. This solution after injection into a biological system could be used to extend the pharmacokinetics of the drug profile, thus creating a sustained release profile in patients.

Some of the delivery method applies into the eye that follows the local dose generates sufficient therapeutically effective dose of the direct factor Xa inhibitor at day 1 through day 72. Some of the delivery method generates sufficient therapeutically effective dose of the direct factor Xa inhibitor lasting through 30 days locally, 60 days, 90 days, and even 6 months while negligible in the systemic circulation at day 1, at day 2, at day 3, at day 7, and even longer.

### Example 53: Preparation of Directfactor Xa Inhibitor Apixaban and Direct Factor IIa Inhibitor Argatroban Crystal Fine Particles Composition in PEG 300 for Ophthalmic Injection

In a similar formulation as in Example 52, combined Anticoagulant solution of apixaban and argatroban was created by taking 400 uL of the 6200 ug/mL clear supernatant of apixaban from example 52, and adding it to a vial containing 2.44 mg of argatroban. The solution was sonicated 10 minutes until clear. The clear solution was analyzed by HPLC and the concentration was found to be 6200 ug/mL apixaban and 5690ug/mL argatroban. This clear solution is ready for eye injection.. After injection into eyes, these drugs will form a crystal fine particle in PEG 300 and the drug has sustained release profiles. The crystal fine particles were verified by following.

Similar procedure can be used to verify a crystalline form of anticoagulant apixaban and argatroban as in Example 23.

This clear periocular injection comprises fine particles containing Apixaban and Argatroban and enables both anticoagulant deliver to the posterior segments. The drug can be efficiently delivered to the posterior segments (such as a retina, a choroid and an optic nerve) while scarcely injuring ophthalmic tissues by administering the fine particles containing the drug periocularlly. This solution after injection into a biological system could be used to extend the pharmacokinetics of the drug profile, thus creating a sustained release profile in patients.

Some of the delivery method applies into the eye that follows the local dose generates sufficient therapeutically effective dose of the direct factor Xa inhibitor and/or the director factor IIa inhibitor at day 1 through day 72. Some of the delivery method generates sufficient therapeutically effective dose of the direct factor Xa inhibitor and/or director factor IIa inhibitor lasting through 30 days locally, 60 days, 90 days, and even 6 months while below the systemic circulation therapeutic level at day 1, at day 2, at day 3, or at day 7.

### Example 54: Preparation of Ranibizumab and Apixaban in Port Delivery System for Sustained Release of Drug for Ophthalmic Applications

The therapeutic agent delivery device (port delivery system) combined with a formulation containing Ranibizumab and Apixaban offers a unique advantage of Ranibizumab and Apixaban to release therapeutic ingredients for extended periods of time and controlled release. The formulations described in Example 24 to Example 27 and Example 52 to Example 53 can be combined with Ranibizumab and Apixaban in the sustained release device(port delivery system). The drugs can be injected separately to the eye while the device is implanted or combined into solution vials with Ranibizumab. Further, long-acting drug delivery system with the potential to reduce treatment burden while maintaining optimal vision outcomes by enabling the continuous delivery of a customized formulation of ranibizumab and apixaban into the target tissue. The range of concentration in formulations is adjustable.

Equal parts of a solution of containing 40% Captisol and 3200 ug/mL Apixaban can be combined with 100 mg/mL Ranibizumab solution to create 1600 ug/mL Apixaban and 50 mg/mL Ranibizumab. The solution is filtered, and pH is measured and adjusted to the suitable physiological pH’s. then fill into the port delivery system sustained release device.

Therapeutic agent release testing is performed by measuring the amount of therapeutic agent released by the port delivery system into a fluid representative of vitreous, maintained at 37° C. in an incubator. The port delivery system is suspended in a container containing phosphate buffered saline. Periodically, the port delivery system is transferred into a new container and the concentration of therapeutic agent is measured in the fluid of the previous container. Rates are calculated from the amount of therapeutic agent released divided by the sample collection duration. The percent cumulative release is calculated from the cumulative amount of therapeutic agent divided by the amount of therapeutic agent initially filled into the therapeutic device (port delivery system).

The therapeutic agent delivery device (port delivery system) combined with a formulation containing Ranibizumab and Apixaban into the eye that follows the local dose generates sufficient therapeutically effective dose lasting through 1 day, 7 days, 14 days, 30 days locally, 60 days, 90 days, 6 months or even longer while negligible in the systemic circulation. Extended or sustained release is achieved with the therapeutic agent released from the therapeutic device after implantation.

### Example 55: Preparation of Ranibizumab and Argatroban in Port Delivery System for Sustained Release of Drug for Ophthalmic Applications

The therapeutic agent delivery device (port delivery system) combined with a formulation containing Ranibizumab and Argatroban offers a unique advantage of Ranibizumab and Apixaban to release therapeutic ingredients for extended periods of time and controlled release. The formulations described in Example 53 can also be combined with Ranibizumab and Argatroban in the sustained release device(port delivery system). The drugs can be injected separately to the eye while the device is implanted or combined into solution vials with Ranibizumab and Argatroban. Further, long-acting drug delivery system with the potential to reduce treatment burden while maintaining optimal vision outcomes by enabling the continuous delivery of a customized formulation of ranibizumab and Argatroban into the target tissue. The range of concentration in formulations is adjustable.

Equal parts of a solution of containing 40% Captisol and 5000 ug/mL Argatroban can be combined with Ranibizumab 100 mg/mL solution to create 1600 ug/mL apixaban and 50 mg/mL Ranibizumab before instilling into the port delivery system reservoir chamber sustained delivery system.

Therapeutic agent release testing is performed by measuring the amount of therapeutic agent released by the port delivery system into a fluid representative of vitreous, maintained at 37° C. in an incubator. The port delivery system is suspended in a container containing phosphate buffered saline. Periodically, the port delivery system is transferred into a new container and the concentration of therapeutic agent is measured in the fluid of the previous container. Rates are calculated from the amount of therapeutic agent released divided by the sample collection duration. The percent cumulative release is calculated from the cumulative amount of therapeutic agent divided by the amount of therapeutic agent initially filled into the therapeutic device (port delivery system).

The therapeutic agent delivery device (port delivery system) combined with a formulation containing Ranibizumab and Argatroban into the eye that follows the local dose generates sufficient therapeutically effective dose lasting through 1 day, 7 days, 14 days, 30 days locally, 60 days, 90 days, 6 months or even longer while negligible in the systemic circulation. Extended or sustained release is achieved with the therapeutic agent released from the therapeutic device after implantation.

### Example 56: Preparation of Ranibizumab and Apixaban and Arzatroban in Port Delivery System for Sustained Release of Drug for Ophthalmic Applications

The therapeutic agent delivery device (port delivery system) combined with a formulation containing Ranibizumab and Apixaban and Argatroban also offers a unique advantage over all previous applications of Ranibizumab and Apixaban and Argatroban to release therapeutic ingredients for extended periods of time and controlled release. The formulations described in Example 53 to Example 54 can also be combined with Ranibizumab and Apixaban and Argatroban in the sustained release device(port delivery system). The drugs can be injected separately to the eye while the device is implanted or combined into solution vials with Ranibizumab. Further, long-acting drug delivery system with the potential to reduce treatment burden while maintaining optimal vision outcomes by enabling the continuous delivery of a customized formulation of ranibizumab and apixaban and Argatroban into the target tissue. The range of concentration in formulations is adjustable.

Equal parts of a solution of containing 40% Captisol and 3200 ug/mL Apixaban and 5000 ug/mL Argatroban can be combined with Ranibizumab 100 mg/mL solution to create 1600 ug/mL Apixaban 2500 ug/mL Argatroban and 50 mg/mL Ranibizumab before instilling into the port delivery system reservoir chamber sustained release device.

Therapeutic agent release testing is performed by measuring the amount of therapeutic agent released by the port delivery system into a fluid representative of vitreous, maintained at 37° C. in an incubator. The port delivery system is suspended in a container containing phosphate buffered saline. Periodically, the port delivery system is transferred into a new container and the concentration of therapeutic agent is measured in the fluid of the previous container. Rates are calculated from the amount of therapeutic agent released divided by the sample collection duration. The percent cumulative release is calculated from the cumulative amount of therapeutic agent divided by the amount of therapeutic agent initially filled into the therapeutic device (port delivery system).

The therapeutic agent delivery device (port delivery system) combined with a formulation containing Ranibizumab and Apixaban and Argatroban into the eye that follows the local dose generates sufficient therapeutically effective dose lasting through 1 day, 7 days, 14 days, 30 days locally, 60 days, 90 days, 6 months or even longer while below the systemic circulation therapeutic level. Extended or sustained release is achieved with the therapeutic agent released from the therapeutic device after implantation.

### Example 57: Preparation of Direct Factor IIa Inhibitor Injection Formulation for Ophthalmic Application

Direct factor IIa inhibitor (such as Argatroban etc.) at target concentration in USP proof alcohol dissolves in PEG and/or copolymer of PEG with USP proof alcohol. The drug solution is injected (10 to 20 µL) into eye. This formulation, following local application into the eye, is sufficient to provide a therapeutically effective dose of the direct factor IIa inhibitor at day 1 through day 72 or more. Some formulations are sufficient to generate a therapeutically effective dose of the direct factor IIa inhibitor lasting through 30 days locally, 60 days, 90 days, and even 6 months while below in the systemic circulation therapeutic levels at or after day 1, at day 2, at day 3, at day 7, from introduction in the eye.

### Example 58: Preparation of Direct Factor IIa Inhibitor Suspension Formulation for Ophthalmic Application With Excipients

Direct factor IIa inhibitor (such as Argatroban etc.) at target concentration formulated as ophthalmic suspension or solution that contains excipients (all, less, or more) such as suspending agents carboxymethylcellulose, hypromellose, povidone, buffers, preservatives, surfactants, caffeine, nicotinamide derivatives and complex/encapsulating agents or derivatives of cyclodextrin (hydroxylpropyl cyclodextrin, captisol). This formulation can use as short release or longer release depending on the delivery method. Some of the delivery methods are sufficient to generate a therapeutically effective dose of the direct factor IIa inhibitor lasting through 1 hour, 1 day, 30 days locally, 60 days, 90 days, and even 6 months while being below the systemic circulation therapeutic levels at day 1, at day 2, at day 3, at day 7.

### Example 59: Preparation of Argatroban Combined With 40% Capitsol in Citrate for Application in Ophthalmic Injection or Eye Drops

10,560 mg Capitsol was dissolved in 15 mM sodium citrate in a 25.0 mL volumetric flask, and the solution was sonicated and mixed until clear. This solution labeled as 40% (400 mg/mL) Captisol(Solution E). 51 mg of argatroban was weighed into a separate vial and 10.0 mL of the 40% Captisol solution(Solution E) prepared above was added. This 10 mL of argatroban solution was sonicated with heat at 40° C. for approximately 30 minutes to maximally dissolve the argatroban. This “saturated argatroban solution” was cooled and filtered to create a clear solution of argatroban in 40% Captisol (Solution H) and pH is measured and adjusted to the suitable physiological pH’s. The concentration of the anticoagulants was 5.0 mg/mL of argatroban. These drug solutions are injected (10 to 50 µL) into rabbit’s eye or swine’s eye with the drug concentrations in target eye tissues and whole blood. Drug concentrations in the eye tissues and in the blood is measured. In addition, ocular pressure is measured during study.

If formulated as eye drops, other than preservatives, the other inactive ingredient may be added to adjust drug concentration and tonicity such as buffers for adjusting pH, drug stabilizers such as anti-oxidants, solubility enhancers, viscosity adjusters, and the like. These inactive ingredients include but not limit to benzalkonium chloride, chlorobutanol, sodium dehydroacetate, phenethyl alcohol, benzethonium chloride, and the like; Boric Acid, Edetate Disodium, Potassium Chloride, Sodium Borate, Sodium Chloride and the like; methyl cellulose, hydroxyethyl cellulose, carboxymethyl cellulose, hydroxypropyl methyl cellulose, polyvinyl alcohol and the like; polyoxyethylene hydrogenated castor oil, polyethylene glycol, polysorbate 80, polyoxyethylene monostearate and the like; sodium edetate and citric acid, sodium edetate and sodium bisulfite and the like. This formulation applies into the eye that follows the local dose generates sufficient therapeutically effective dose of the director factor IIa inhibitor while below the systemic circulation therapeutic levels.

### Example 60: Preparation of Organic Formulation Containing Direct Factor Xa Inhibitor Apixaban and Direct Factor IIa Inhibitor Argatroban for Application in Ophthalmic Injection

250 mg of Apixaban is weighed into a 20 mL scintillation vial. To the same vial, 518.13 mg of Argatroban is added, and 10 mL of Dimethyl sulfoxide (DMSO) is added to the vial containing weighed drug. Mix thoroughly by applying stirring, shaking, sonication. Allow the solution to equilibrate to room temperature. Filter the formulation through a PTFE membrane filter. Transfer the filtered solution into sterilized storage vials. The concentration of the anticoagulants is measured as 25.0 mg/mL of Apixaban and 50.0 mg/mL of Argatroban. These drug solutions are injected (10 to 50 µL) into rabbit’s eye or swine’s eye with the drug concentrations in target eye tissues and whole blood. Drug concentrations in the eye tissues and in the blood is measured. In addition, ocular pressure is measured during study. This formulation applies into the eye that follows the local dose generates sufficient therapeutically effective dose of the drug while below the systemic circulation therapeutic level.

### Example 61: Preparation of Organic Formulation Containing Direct Factor Xa Inhibitor Apixaban and Directfactor IIa Inhibitor Argatroban and m-TOR Sirolimus for Application in Ophthalmic Injection

125 mg of Apixaban is weighed into a 20 mL scintillation vial. To the same vial, 259.07 mg of Argatroban and 125 mg of Sirolimus are added, and 5 mL of Dimethyl sulfoxide (DMSO) is added to the vial containing weighed drug. Mix thoroughly by applying stirring, shaking, sonication. Allow the solution to equilibrate to room temperature. Filter the formulation through a PTFE membrane filter. Transfer the filtered solution into sterilized storage vials. The concentration of the drugs is measured as 25.0 mg/mL of Apixaban and 50.0 mg/mL of Argatroban and 25.0 mg/mL of Sirolimus. These drug solutions are injected (10 to 50 µL) into rabbit’s eye or swine’s eye with the drug concentrations in target eye tissues and whole blood. Drug concentrations in the eye tissues and in the blood is measured. In addition, ocular pressure is measured during study. This formulation applies into the eye that follows the local dose generates sufficient therapeutically effective dose of the drug while below the systemic circulation therapeutic level.

### Example 62: Preparation of Tissue Plasminogen Activator (tPA) Formulation for Application in Ophthalmic Eye Drops or Injection

The tPA therapy could be effective for the rapid dissolution of fibrin clots in the anterior chamber of the human eye, as well as for lysis of fibrin clots after vitrectomy and failed blebs after glaucoma filtering surgery or wet age-related macular degeneration (AMD). The tissue plasminogen activator (tPA) eye drops administered topically to patients with postoperative hyphema could give rapid clearance of the clot. Intracameral tPA injection is a safe and effective way to help lyse fibrin and dissolve blood clots to reopen occluded glaucoma drainage devices. tPA is a nonsurgical eye injection that’s often used to treat patients suffering from wet age-related macular degeneration (AMD). It’s also used to treat a common side effect of AMD, submacular hemorrhages. An amount of 25 mg or more of tPA has been widely used intracamerally or intravitreally. The tPA therapeutic agent can be, for example, tPA, a tPA derivative, a small molecule direct or indirect tPA agonist, or a gene therapy vector. The most common used ones are recombinant tissue plasminogen activators (r-tPA) which are manufactured in labs. Examples of these commercial drugs include alteplase, reteplase, and tenecteplase.

The tPA eye drops is prepared by reconstitution of 0.5 mg of lyophilised tissue plasminogen activator (The commercially available r-TPA such as alteplase or reteplase or Tenecteplase) in 0.5 ml of sterile water according to the manufacturer’s recommendations. The reconstituted r-TPA is diluted 1:4 in sterile balanced salt solution. Using balanced salt solution, tPA is diluted to achieve a 250 µg per mL concentration. The eyedrops are then stored at -70° C. in an ultralow freezer and before use. The eye drops are thawed to room temperature when use.

The tPA eye injection is prepared by reconstitution of 1 mg of lyophilised tissue plasminogen activator (The commercially available r-TPA such as alteplase or reteplase or Tenecteplase) in 1 ml of sterile water according to the manufacturer’s recommendations. All preparation is under a sterile hood. The reconstituted r-TPA is diluted 1:4 in sterile balanced salt solution which was then divided into multiple aliquots of 1 ml (250 µg) of r-TPA. Using balanced salt solution, tPA is diluted to achieve a 250 µg per mL concentration. The syringes are then stored at -70° C. in an ultralow freezer and before use the syringes are thawed to room temperature.

The injections are performed with the patient in the supine position with a lid speculum inserted to separate the lids. A sterile 30 gauge cannula is placed on the syringe and 0.1 ml (25 µg) r-TPA is injected into the anterior chamber. Slit lamp examination was performed postoperatively after 2 hours, 1 day, 2 days, 3 days, and then weekly to assess complications and dissolution of the fibrin. Visual acuity was measured by Snellen or Sheridan-Gardiner acuity tests depending on the age of the patient.

tPA use could have prevented the need for additional glaucoma surgery. One or more injections of tPA may have been used to clear or prevent tube blockage by blood and/or fibrin to result in a successful ocular outcome, or decrease eye pressure.

The tPA eye drops or injections can benefit eye disease such as vitreous opacity, wet age-related macular degeneration (AMD), glaucoma and anterior chamber fibrinoid syndrome after cataract extraction etc.

### Example 63: Preparation of Tissue Plasminogen Activator (tPA) and Direct Factor Xa Inhibitor Apixaban and Direct Factor IIa Inhibitor Argatroban in Captisol Eye Drops for Ophthalmic Application

The tPA eye drops is prepared by reconstitution of 0.5 mg of lyophilised tissue plasminogen activator (The commercially available r-TPA such as alteplase or reteplase or Tenecteplase) in 0.5 ml of sterile water according to the manufacturer’s recommendations. The reconstituted r-TPA is diluted 1:4 in sterile balanced salt solution. Using balanced salt solution, tPA is diluted to achieve a 250 µg per mL concentration. The eyedrops are then stored at -70° C. in an ultralow freezer and before use. The eye drops are thawed to room temperature when use.

The direct factor Xa inhibitor Apixaban and direct factor IIa inhibitor Argatroban in captisol eye drops was prepared as below: 10,560 mg Capitsol was dissolved in 15 mM sodium citrate in a 25.0 mL volumetric flask to make 40% Captisol in citrate buffer. 100.2 mg of apixaban was weighed into a separate vial and 20.0 mL of the 40% Captisol citrate buffer solution prepared above was added. This 20 mL of apixaban solution was sonicated with heat at 40° C. for approximately 30 minutes to maximally dissolve the apixaban. This “saturated apixaban solution” was cooled and filtered to create a clear solution of apixaban in 40% Captisol. 51 mg of argatroban is weighed and added into the same vial, and finally 10 mL of the saturated apixaban solution prepared above is added to the same vial. The final 2 drug combination solution is sonicated with heat if needed until clear. The cooled solution is filtered, and pH is measured and adjusted to the suitable physiological pH’s. The concentration of the anticoagulants are projected to be at 3.2 mg/mL of apixaban and 5.0 mg/mL of argatroban. The eye drops, other than preservatives, the other inactive ingredient may be added to adjust drug concentration and tonicity such as buffers for adjusting pH, drug stabilizers such as anti-oxidants, solubility enhancers, viscosity adjusters, and the like.

The tPA eye drops and the direct factor Xa inhibitor Apixaban and direct factor IIa inhibitor Argatroban eye drops can apply in alternative time to clear fibrin in eyes over times.

### Example 64: Preparation of Tissue Plasminogen Activator (tPA) and Direct Factor Xa Inhibitor Apixaban Crystal Fine Particle in PEG 300 Formulation for Application in Ophthalmic Injection With Burst Release of tPA and Sustained Release of Direct Factor Xa Inhibitor Apixaban

The tPA eye injection is prepared by reconstitution of 1 mg of lyophilised tissue plasminogen activator (The commercially available r-TPA such as alteplase or reteplase or Tenecteplase) in 1 ml of sterile water according to the manufacturer’s recommendations. All preparation is under a sterile hood. The reconstituted r-TPA is diluted 1:4 in sterile balanced salt solution which was then divided into multiple aliquots of 1 ml (250 µg) of r-TPA. Using balanced salt solution, tPA is diluted to achieve a 250 µg per mL concentration. The syringes are then stored at -70° C. in an ultralow freezer and before use the syringes are thawed to room temperature. This tPA injection can be efficiently delivered to the posterior segments and could be created a burst release profile in patients to dissolve/dissolute most of fibrin in eyes.

The direct factor Xa inhibitor Apixaban crystal fine particle in PEG 300 was prepared as below: 10 mg of apixaban powder was weighed into a vial and 50 µL of 190 proof USP grade ethanol was added first in the same vial, then 950 uL of PEG 300 (polyethylene glycol) was added to solubilize the drug. The vial was sonicated with 40° C. heat for 30 minutes and then was placed in the 40° C. oven overnight for 20 hours. The cloudy solution was transferred to a polyethylene centrifuge tube and centrifuged for 15 minutes to pelletize the undissolved apixaban. The clear supernatant was analyzed by HPLC and the concentration was found to be 6200 µg/mL apixaban in 95% PEG 300 solution. This clear solution is ready for eye injection. After injection into eyes, these drugs will form a crystal fine particle in PEG 300 and the drug has sustained release profiles. The crystal fine particles were verified by following.

100 µL of the clear PEG 300 solution was added to a vial with 4.3 mL of Dulbecco’s phosphate buffered saline to mimic the ophthalmic vitreous humor. The solution immediately cleared upon mixing. The solution stands overnight for 24 hours at room temperature. Upon close inspection, the apixaban had precipitated producing a small carpet of crystals on the bottom of the glass vial. The solution was then centrifuged, and the supernatant was analyzed by HPLC. The concentration was found to be 94 µg/mL of apixaban compared to the theoretical concentration in the PBS at 141 µg/mL. Therefore 67% of the apixaban was in solution and 33 % had precipitated out to crystalline form.

This clear periocular injection comprises fine particles containing Apixaban and enables Apixaban to deliver to the posterior segments. Apixaban can be efficiently delivered to the posterior segments and could be used to extend the pharmacokinetics of the drug profile, thus creating a sustained release profile in patients and continue to clear fibrin in eyes over times.

tPA injection apply first then follows apixaban eye injections in a proper time interval. These ophthalmic injections with burst release of tPA and sustained release of direct factor Xa inhibitor Apixaban to clear the fibrin in eyes over time.

### Example 65: Preparation of Tissue Plasminogen Activator (tPA) and Direct Factor Xa Inhibitor Apixaban and Direct Factor IIa Inhibitor Argatroban Crystal Fine Particle in PEG 300 Formulation for Application in Ophthalmic Injection with Burst Release of tPA and Sustained Release of Direct Factor Xa Inhibitor Apixaban and Direct Factor IIa Inhibitor Argatroban

The tPA eye injection is prepared by reconstitution of 1 mg of lyophilised tissue plasminogen activator (The commercially available r-TPA such as alteplase or reteplase or Tenecteplase) in 1 ml of sterile water according to the manufacturer’s recommendations. All preparation is under a sterile hood. The reconstituted r-TPA is diluted 1:4 in sterile balanced salt solution which was then divided into multiple aliquots of 1 ml (250 µg) of r-TPA. Using balanced salt solution, tPA is diluted to achieve a 250 µg per mL concentration. The syringes are then stored at -70° C. in an ultralow freezer and before use the syringes are thawed to room temperature. This tPA injection can be efficiently delivered to the posterior segments and could be created a burst release profile in patients to dissolve/dissolute most of fibrin in eyes.

The direct factor Xa inhibitor Apixaban and direct factor IIa inhibitor Argatroban crystal fine particle in PEG 300 were prepared as below: In a similar formulation as in Example 64, combined Anticoagulant solution of apixaban and argatroban was created by taking 400 uL of the 6200 ug/mL clear supernatant of apixaban from example 64, and adding it to a vial containing 2.44 mg of argatroban. The solution was sonicated 10 minutes until clear. The clear solution was analyzed by HPLC and the concentration was found to be 6200 ug/mL apixaban and 5690 ug/mL argatroban. This clear solution is ready for eye injection. After injection into eyes, these drugs will form a crystal fine particle in PEG 300 and the drug has extended-release profiles. The crystal fine particles were verified by following.

Similar procedure can be used to create a crystalline form of anticoagulant apixaban and argatroban as in Example 64. This clear periocular injection comprises fine particles containing Apixaban and Argatroban and enables both anticoagulant deliver to the posterior segments.

tPA, Apixaban and Argatroban can be efficiently delivered to the posterior segments and could be used to extend the pharmacokinetics of the drug profile, thus creating a sustained release profile in patients and continue to clear fibrin in eyes over times.

tPA injection apply first then follows anticoagulant eye injection in a proper time interval. These ophthalmic injections with burst release of tPA and sustained release of direct factor Xa inhibitor Apixaban and direct factor IIa inhibitor to clear the fibrin in eyes over time.

### Example 66: Preparation of Direct Factor Xa Inhibitor Apixaban and Direct Factor IIa Inhibitor Argatroban Eve Drops and Crystal Fine Particle Eye Injection Formulation With Burst Release and Sustained Release of Direct Factor Xa Inhibitor Apixaban and Direct Factor IIa Inhibitor Argatroban

The direct factor Xa inhibitor Apixaban and direct factor IIa inhibitor Argatroban in captisol eye drops was prepared as in example 63.

The direct factor Xa inhibitor Apixaban and direct factor IIa inhibitor Argatroban crystal fine particle in PEG 300 were prepared as in Example 64. The eye injection solution was analyzed by HPLC and the concentration was found to be 6200 ug/mL apixaban and 5690 ug/mL argatroban. This clear solution is ready for eye injection.. After injection into eyes, these drugs will form a crystal fine particle in PEG 300 and the drug has sustained release profiles. The crystal fine particles were verified by following.

Similar procedure can be used to create a crystalline form of anticoagulant apixaban and argatroban as in Example 64. This clear periocular injection comprises Apixaban and Argatroban and enables both anticoagulant deliver to the posterior segments to form crstal fine particles after injection with extended-release profiles. Apixaban and Argatroban eye drops can be efficiently delivered to drug and have a burst release profile in patients. Apixaban and Argatroban eye injection can be efficiently delivered to the posterior segments and could be used to extend the pharmacokinetics of the drug profile, thus creating a sustained release profile in patients. The direct factor Xa inhibitor Apixaban and direct factor IIa inhibitor Argatroban eye drops apply first then follows by anticoagulant the direct factor Xa inhibitor Apixaban and direct factor IIa inhibitor Argatroban eye injection in a proper time interval. These eye drops/injections have a burst release of the direct factor Xa inhibitor Apixaban and direct factor IIa inhibitor Argatroban and a sustained release of direct factor Xa inhibitor Apixaban and direct factor IIa inhibitor to clear the fibrin in eyes over time.

### Example 67: Preparation of Direct Factor Xa Inhibitor Apixaban and Direct Factor IIa Inhibitor Argatroban Drug Coated Microneedle Implant for Sustained Release of Direct Factor Xa Inhibitor Apixaban and Direct Factor IIa Inhibitor Arzatroban

The direct factor Xa inhibitor Apixaban and direct factor IIa inhibitor Argatroban can be coated in a metal microneedle. The metal microneedle is fabricated by laser cut from 50-80 micron thick stainless steel sheet following an acid electropolishing. The final size of microneedle is 400 µm in length and 40×100 µm in cross section with 40-50° in tip angle to avoid penetration through eye corneal tissue.

The microneedle can optionally undergo surface treatment if the surface is not porous (i.e. plasma treatment or other friction surface treatment). After surface treatment, the coating could be spray coated or dip coated.

When spray coated, a microprocessor controlled ultrasonic sprayer is used to coat the microneedle containing drug/excipient solution to the entire surface of the implant. After coating, the microneedle is placed in a 70° C. oven for about 2 hours to remove the residue solvent. The microneedle is then put in a coil and packaged. The pouches were sterilized.

The microneedle implant provides a sustained release of direct factor Xa inhibitor Apixaban and direct factor IIa inhibitor Argatroban. This microneedle implant may use for treatment of inflammation or fibrin related of uveitis, glaucoma, diabetic macular edema, age-related macular degradation, corneal infection, cytomegalovirus retinitis or anterior chamber fibrinoid syndrome after cataract extraction etc.

### Example 68: Preparation of the Complement Factor C3 Inhibitor Eye Injection for Ocular Inflammation

Complement is the main driver of disease for AMD, Uveitis, Glaucoma. Local inhibition of complement activation has been considered a promising approach for treating these diseases. Complement pathway has three pathways which involves various factors, B, D, H & I, which interact with each other, and with C3b, to form a C3 convertase, C3bBb, that can activate more C3. C3 is the converging point for all 3 complement pathways and involves in the amplification loop.

Compstatin is a C3 inhibitor and could be a potent inhibitor, blocking all three complement systems. A fourth generation Compstatin complement C3 inhibitor (Cp40-KKK, AMY106) can block C3 for at least 3 months with a single intravitreal injection. Compstatin and its analogs(Cp40-KKK, AMY106) are synthesized in a peptide synthesizer using F-moc amide resin (4-(2′,4′-dimethoxyphenyl-F-moc-aminomethyl)-phenoxy resin). Cp40-KKK or AMY106 eye injection is prepared by reconstitution of 100 mg of Cp40-KKK or AMY106 in 1 ml of Dulbecco’s phosphate buffered saline. All preparation is under a sterile hood. Using balanced salt solution, the syringes are then stored at -70° C. in an ultralow freezer and before use the syringes are thawed to room temperature. The injections are performed with the patient in the supine position with a lid speculum inserted to separate the lids. A sterile 30 gauge cannula is placed on the syringe and 0.1 ml (10 mg of Cp40-KKK or AMY106) of solution will be injected. The above solution is injected into the anterior chamber. Compstatin or its analogs eye injections can benefit eye disease such as AMD, Uveitis, Glaucoma, etc.

### Example 69: Preparation of the Complement Factor C3 Inhibitor Compstatin or Its Analog and Direct Factor Xa Inhibitor Apixaban and Direct Factor IIa Inhibitor Argatroban in PEG 300 Eye Injection for Ocular Inflammation

The combination of complement factor C3 inhibitor Compstatin or its analog and direct factor Xa inhibitor Apixaban and direct factor IIa inhibitor Argatroban in PEG 300 for eye injection would be a powerful therapy for AMD, Uveitis, Glaucoma and other ocular inflammation.

Cp40-KKK or AMY106 eye injection is prepared by reconstitution of 200 mg of Cp40-KKK or AMY106 in 1 ml of Dulbecco’s phosphate buffered saline. All preparation is under a sterile hood. Using balanced salt solution, the syringes are then stored at -70° C. in an ultralow freezer and before use the syringes are thawed to room temperature.

The direct factor Xa inhibitor Apixaban and direct factor IIa inhibitor Argatroban in PEG 300 was prepared as below: 15 mg of apixaban powder 10 mg of Argatroban powder were weighed into a vial and 50 µL of 190 proof USP grade ethanol was added first in the same vial, then 950 uL of PEG 300 (polyethylene glycol) was added to solubilize the drug. The vial was sonicated with 40° C. heat for 30 minutes and then was placed in the 40° C. oven overnight for 20 hours. The cloudy solution was transferred to a polyethylene centrifuge tube and centrifuged for 15 minutes to pelletize the undissolved apixaban. The clear supernatant was analyzed by HPLC and the concentration was found to be 5299 µg/mL Apixaban and 10363 ug/mL Argatroban in 95% PEG 300 solution.

100 µL of the clear direct factor Xa inhibitor Apixaban and direct factor IIa inhibitor Argatroban PEG 300 solution and 100 µL of Cp40-KKK or AMY106(200 mg/ml) of Dulbecco’s phosphate buffered saline solution are diluted to 200 µL of Dulbecco’s phosphate buffered saline to mimic the ophthalmic vitreous humor. The solution immediately will be cleared upon mixing and is ready for injection.

The injections are performed with the patient in the supine position with a lid speculum inserted to separate the lids. A sterile 30-gauge cannula is placed on the syringe and 0.1 ml (25 µg of Apixaban, 50 µg of Argatroban,10 mg of Cp40-KKK or AMY106) of solution will be injected. The above solution is injected into the anterior chamber.

Optionally, direct factor Xa inhibitor Apixaban and direct factor IIa inhibitor Argatroban PEG 300 and C3 inhibitor can be administrated separately. For example, AMY106 can be injected monthly; direct factor Xa inhibitor Apixaban and direct factor IIa inhibitor Argatroban PEG 300 can be injected weekly.

After injection into eyes, these drugs will form a crystal fine particles in PEG 300 and the drug has extended release profiles. The complement factor C3 inhibitor Compstatin or its analog and direct factor Xa inhibitor Apixaban and direct factor IIa inhibitor Argatroban in PEG 300 can benefit eye disease such as AMD, Uveitis, Glaucoma, etc. The rate of progression of atrophy in the eyes of patients with dry AMD might be slowed down or stopped with the combination of the complement factor C3 inhibitor Compstatin or its analog and direct factor Xa inhibitor Apixaban and direct factor IIa inhibitor Argatroban in PEG 300 eye injection.

### Example 70: Preparation of the Complement Factor C5 Inhibitor and Direct Factor Xa inhibitor Apixaban and Direct Factor IIa Inhibitor Argatroban in PEG 300 Eye Injection for Ocular Inflammation

Complement is the main driver of disease for AMD, Uveitis, Glaucoma. Local inhibition of complement activation has been considered a promising approach for treating this disease. Immunosuppressive drug such as Eculizumab or Ravulizumab specifically binds to the terminal complement component 5, or C5, which acts at a late stage in the complement cascade. When activated, C5 is involved in activating host cells, thereby attracting pro-inflammatory immune cells, while also destroying cells by triggering pore formation. By inhibiting the complement cascade at this point, the normal, disease-preventing functions of proximal complement system are largely preserved, while the properties of C5 that promote inflammation and cell destruction are impeded.

The complement factor C5 inhibitor eye injection is prepared with the commercially available Eculizumab or Ravulizumab according to the manufacturer’s recommendations and is diluted with commercially available Saline if needed. All preparation is under a sterile hood. The dose is range from 600 mg to 1200 mg weekly.

The direct factor Xa inhibitor Apixaban and direct factor IIa inhibitor Argatroban in PEG 300 was prepared as in example 69. The dose is range from 25 µg to 0.5 mg of Apixaban and 50 µg to 1 mg of Argatroban. The injections are performed with the patient in the supine position with a lid speculum inserted to separate the lids. A sterile 30 gauge cannula is placed on the syringe. The above solution is injected into the anterior chamber.

Optionally, direct factor Xa inhibitor Apixaban and direct factor IIa inhibitor Argatroban PEG 300 and C3 inhibitor can be administrated separately. For example, Eculizumab or Ravulizumab can be injected biweekly; direct factor Xa inhibitor Apixaban and direct factor IIa inhibitor Argatroban PEG 300 can be injected weekly.

After injection into eyes, these drugs will form a crystal fine particle in PEG 300 and the drug has extended-release profiles. The above eye injections can benefit eye disease such as AMD, Uveitis, Glaucoma, etc. The rate of progression of atrophy in the eyes of patients with dry AMD might be slowed with combination of Eculizumab or Ravulizumab and direct factor Xa inhibitor Apixaban and direct factor IIa inhibitor Argatroban.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached, or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the present disclosure. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature’s relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present disclosure.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/- 0.1% of the stated value (or range of values), +/- 1% of the stated value (or range of values), +/- 2% of the stated value (or range of values), +/- 5% of the stated value (or range of values), +/- 10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating an ophthalmic condition or disease or an inflammatory ophthalmic condition or disease in a patient, the method comprising:
providing a therapeutic composition comprising at least one of a direct factor Xa inhibitor and a factor IIa inhibitor; and
delivering a therapeutically effective dose of the therapeutic composition to the patient’s eye.

2. The method of claim 1, wherein delivering the therapeutically effective dose of the therapeutic composition comprises administering the therapeutically effective dose via injection, an implant, an eye drop, an emulsion, a reservoir, a suspension, an ointment, a nanomicelle, a nanoparticle, a nanosuspension, a liposome, an in-situ gel, a contact lens, or a microneedle.

3. The method of claim 1, wherein the inflammatory ophthalmic condition or disease comprises one or more of angiogenesis, leaking blood vessel, protein deposition, fibrosis, extracellular matrix (ECM) deposition, thrombin generation, clot formation, or fibrin formation, wet age-related macular degeneration (AMD), dry AMD, diabetic retinopathy, glaucoma, uveitis, cataracts, conjunctivitis, and dry eye disease.

4. The method of claim 1, wherein therapeutic composition is delivered to a site of the inflammatory ophthalmic condition or disease selected from the group consisting of an eyelid, a meibomian gland, a trabecular meshwork, Schlemm’s canal, a cornea, a sclera, a vitreous, a choroid, a uvea, and a retina.

5. The method of claim 1, wherein therapeutic composition is delivered non-site-specifically to the patient’s eye.

6. The method of claim 1, further comprising diagnosing the patient as having the inflammatory ophthalmic condition or disease prior to delivering the therapeutically effective dose of the therapeutic composition.

7. The method of claim 1, wherein the therapeutic composition includes at least a direct factor Xa inhibitor is selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-l-phenylethyl)-lh-indole-6-carboxamide(LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), eribaxaban (PD 0348292), 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid(PD0313052), (S)-3-(7-carbamimidoylnaphthalen-2-yl)-2-(4-(((S)-l-(l-iminoethyl)pyrrolidin-3-yl)oxy)phenyl)propanoic acid hydrochloride pentahydrate(DX9065a), letaxaban (TAK-442), GW813893, JTV-803, KFA-144, DPC-423, RPR-209685, MCM-09, or antistasin.

8. The method of claim 7, wherein the direct factor Xa inhibitor comprises rivaroxaban, or a salt, isomer, solvate, analog, derivative, metabolite, or prodrugs thereof.

9. The method of claim 7, wherein the direct factor Xa inhibitor comprises apixaban, or a salt, isomer, solvate, analog, derivative, metabolite, or prodrugs thereof.

10. The method of claim 1, wherein an IC50 of the direct factor Xa inhibitor is within a range of about 0.01 nM to about 1000 nM.

11. The method of claim 10, wherein the IC50 of the direct factor Xa inhibitor is within a range of about 0.1 nM to about 1000 nM.

12. The method of claim 10, wherein the IC50 of the direct factor Xa inhibitor is within a range of about 1 nM to about 1000 nM.

13. The method of claim 10, wherein the IC50 of the direct factor Xa inhibitor is within a range of about 10 nM to about 1000 nM.

14. The method of claim 1, wherein the therapeutically effective dose of the direct factor Xa inhibitor is within a range of about 50 micrograms to about 10 mg.

15. The method of claim 14, wherein the therapeutically effective dose of the direct factor Xa inhibitor is within a range of about 0.1 mg to about 10 mg.

16. The method of claim 14, wherein the therapeutically effective dose of the direct factor Xa inhibitor is within a range of about 1 mg to about 5 mg.

17. The method of claim 1, wherein the therapeutically effective dose is sufficient to generate a tissue concentration of the direct factor Xa inhibitor of about 0.1 ng/g tissue to about 100 mg/g tissue.

18. The method of claim 17, wherein the therapeutically effective dose is sufficient to generate a tissue concentration of the direct factor Xa inhibitor of about 0.2 ng/g tissue to about 100 mg/g tissue.

19. The method of claim 17, wherein the therapeutically effective dose is sufficient to generate a tissue concentration of the direct factor Xa inhibitor of about 0.5 ng/g tissue to about 100 mg/g tissue.

20. The method of claim 17, wherein the therapeutically effective dose is sufficient to generate a tissue concentration of the direct factor Xa inhibitor of about 1 ng/g tissue to about 100 mg/g tissue.

21. The method of claim 17, wherein the therapeutically effective dose is sufficient to generate a tissue concentration of the direct factor Xa inhibitor of about 10 ng/g tissue to about 100 mg/g tissue.

22. The method of claim 17, wherein the therapeutically effective dose is sufficient to generate a tissue concentration of the direct factor Xa inhibitor of about 100 ng/g tissue to about 100 mg/g tissue.

23. The method of claim 17, wherein the therapeutically effective dose is sufficient to generate a blood concentration of the direct factor Xa inhibitor which is less than about 200 ng/ml, 100 ng/ml, 50 ng/ml 25 ng/ml, or 10 ng/ml.

24. The method of claim 17, wherein the therapeutically effective dose is sufficient to generate a blood concentration of the direct factor Xa inhibitor which is less than a systemic therapeutic concentration of the direct factor Xa inhibitor for any systemic indication.

25. The method of claim 17, wherein the therapeutically effective dose is sufficient to generate a blood concentration of the direct factor Xa inhibitor which is smaller than a median maximum serum concentration (Cmax) of the direct factor Xa inhibitor generated by systemic delivery of the direct factor Xa inhibitor to achieve the same tissue concentration at the site of the inflammatory ophthalmic condition or disease.

26. The method of claim 17, wherein the therapeutically effective dose is sufficient to generate a blood concentration of the direct factor Xa inhibitor which does not exceed a median maximum serum concentration (Cmax) of the direct factor Xa inhibitor generated by systemic delivery of the direct factor Xa inhibitor to achieve the same tissue concentration at the site of the inflammatory ophthalmic condition or disease for more than about 6 hours to about 3 days.

27. The method of claim 1, wherein the therapeutically effective dose is sufficient to maintain a tissue concentration of the direct factor Xa inhibitor of about 0.1 ng/g tissue to about 100 mg/g tissue for about 1 day to about 1 year, 30 days to about 1 year, 3 months to about 1 year, or 6 months to about 1 year.

28. The method of claim 1, wherein the therapeutically active substance comprises at least a direct factor IIa inhibitor selected from the group consisting of argatroban, dabigatran, ximelagatran, melagatran, efegatran, inogatran, atecegatran metoxil (AZD-0837), hirudin, hirudin analogs, bivalirudin, desirudin, or lepirudin.

29. The method of claim 28, wherein the direct factor IIa inhibitor comprises argatroban.

30. The method of claim 28, wherein the direct factor IIa inhibitor comprises a salt, isomer, solvate, analog, derivative, metabolite, or prodrugs of argatroban.

31. The method of claim 28, wherein the therapeutically effective dose of the direct factor IIa inhibitor is within a range of about 50 micrograms to about 10 mg.

32. The method of claim 28, wherein the therapeutically effective dose is sufficient to generate a tissue concentration of the direct factor IIa inhibitor of about 0.1 ng/g tissue to about 100 mg/g tissue.

33. A therapeutic composition for inhibiting an inflammatory ophthalmic condition or disease in a patient, the composition comprising:
at least one of a direct factor Xa inhibitor and a factor IIa inhibitor formulated for delivery to an eye of the patient.
